(19) United States
(12) Patent Application Publication
(10) Pub. No.: US 2005/0203010 A1
(43) Pub. Date: Sep. 15, 2005

(54) NOVEL PEPTIDES CONFERRING ENVIRONMENTAL STRESS RESISTANCE AND FUSION PROTEINS INCLUDING SAID PEPTIDES

(75) Inventor: Jong-Sun KIM, Seoul (KR)

Correspondence Address: JHK LAW, P.O. BOX 1078, LA CANADA, CA, 91012-1078, US

(73) Assignee: ATGEN CO., LTD., Seongnam-si, Gyeonggi-do (KR)

(21) Appl. No.: 10/908,400

(22) Filed: May 10, 2005

Related U.S. Application Data
(63) Continuation-in-part of application No. 10/713,851, filed on Nov. 14, 2003.

(30) Foreign Application Priority Data
May 11, 2004 (KR) 10-2004-33123
May 2, 2005 (KR) 10-2005-36882

(51) Int. Cl. A61K 38/17; C07K 14/47

(52) U.S. Cl. 514/12; 530/350

(57) ABSTRACT

The present invention relates to a peptide capable of conferring resistance to environmental stresses, comprising a peptide fragment containing a sequence composed of 10 or more consecutive amino acid residues including five or more acidic amino acid residues, wherein the peptide fragment is derived from the C-terminal acidic tail of synuclein, or its derivative, and to a fusion protein comprising the peptide and a fusion partner protein being linked to the peptide, wherein the fusion protein is resistant to environmental stresses. Also, the present invention is concerned with a method of conferring resistance to environmental stress to a protein of interest, comprising linking the protein to the peptide. While maintaining the intrinsic properties of the fusion partner protein, the fusion protein is resistant to environmental stresses, including heat, pH, metal ions, repeated freezing/thawing and high-concentration of polypeptide.

### FIELD OF THE INVENTION

The present invention relates to a peptide capable of conferring resistance to environmental stresses, comprising a peptide fragment containing a sequence composed of 10 or more consecutive amino acid residues including five or more acidic amino acid residues, wherein the peptide fragment is derived from the C-terminal acidic tail of synuclein, or its derivative, and to a fusion protein comprising the peptide and a fusion partner protein being linked to the peptide, wherein the fusion protein is resistant to environmental stresses. Also, the present invention relates to a method of conferring resistance to environmental stress to a protein of interest, comprising linking the protein to the peptide.

### BACKGROUND OF THE INVENTION

The present invention relates to a peptide capable of conferring resistance to environmental stresses, comprising a peptide fragment containing a sequence composed of 10 or more consecutive amino acid residues including five or more acidic amino acid residues, wherein the peptide fragment is derived from the C-terminal acidic tail synuclein, or its derivative, and to a fusion protein comprising the peptide and a fusion partner protein being linked to the peptide, wherein the fusion protein is resistant to environmental stresses. Also, the present invention relates to a method of conferring resistance to environmental stress to a protein of interest, comprising linking the protein to the peptide.

“Proteins with environmental stress resistance” refer to proteins that physically, chemically and biologically show stability against external environmental factors such as heat, pH, metal ions, organic solvents, etc. Typically among such proteins, there are heat-stable proteins which are stable even at the boiling temperature of water. One group of heat-stable proteins are represented by proteins derived from hyperthermophilic organisms [Jaenicke R. and Bohm G., Curr. Opin. Struct. Bio., 8, 738-748 (1998); Ress D. C. and Adams M. W. W. Structure, 3, 251-254 (1995); and Adams M. W. W., Ann. Rev. Microbiol. 47, 627-658 (1993)]. These proteins have an extremely high melting temperature (hereinafter referred to as “Tm”), relative to their mesophilic counterparts (near or above the boiling point of water). However, when the temperature is increased above the Tm, most hyperthermophilic proteins also denature, leading to insoluble aggregation [Klump et al., J. Biol. Chem., 267, 22681-22685 (1992); Klump et al., Pure. Appl. Chem., 66, 485-489 (1994); Cavagnero S. et al., Biochemistry, 34, 9865-9873 (1995)].

Another group of heat-stable proteins, which have been recently recognized, are the intrinsically unstructured proteins [Plaxco, K. W. and Groβ M., Nature, 386, 657-658 (1997); Wright P. E. and Dyson H. J., J. Mol. Biol., 293, 321-331 (1999)]. The reason why the intrinsically unstructured proteins are heat-stable is because the conformation of the intrinsically unstructured proteins is not extensively changed by heat treatment. Thermodynamically, the intrinsically unstructured proteins are heat resistant proteins (hereinafter referred to as “HRPs”) rather than heat-stable proteins since their conformation almost unfolds at room temperature and is somewhat changed at high temperatures (Kim T. D. et al., Biochemistry, 39, 14839-14846 (2000)). Thus, the term “heat resistant proteins (HRPs)” is more appropriate for describing the thermal behavior of the intrinsically unstructured proteins. That is, HRPs can be defined as proteins that are not aggregated by heat treatment, such as hyperthermophilic proteins and unstructured proteins.

The thermal behavior of proteins was systematically investigated by purifying and characterizing some HRPs that are not aggregated by heat treatment from Jurkat T cells and human serum (Kim T. D. et al., Biochemistry, 39, 14839-14846 (2000)). According to studies on the heat resistance of proteins from Jurkat cell lysates and human serum, four major types of thermal behavior of HRPs were recognized, which are as follows. Group I HRPs are represented by unstructured proteins such as a-synuclein and as-casein, which have a semi-unfolded conformation regardless of temperature. Group II HRPs, represented by human serum fetuin and albumin, are characterized by an irreversible conformational change upon heat treatment. Group III HRPs, represented by transthyretin and bovine serum fetuin, are characterized by a reversible conformational change. Group IV HRPs, conventional heat-stable proteins such as hyperthermophilic proteins, are characterized by the absence of heat induced conformational changes.

Most proteins unfold and in turn precipitate as the temperature increases, and the process is usually irreversible (Bull H. B. and Breese K., Arch. Biochem. Biophys., 156, 604-612 (1973)). The improvement of stress resistance, including the improvement of thermal stability, is one of the tasks to be solved for proteins, such as hormones, cytokines and enzymes, widely used in the medical or industrial fields. Improvement of stress-resistance, of course, renders the life span of products to be elongated, thereby leading to development of novel medical products and more stable industrial enzymes, foods or chemical products. Therefore, the present invention relating to novel stress-resistant proteins will be very useful.

Leading to the present invention, the research into properties of the proteins stable against environmental stresses such as heat, pH, metal ions, etc., conducted by the present inventors, resulted in the finding that a peptide comprising a peptide fragment containing a sequence composed of 10 or more consecutive amino acid residues including five or more acidic amino acid residues, wherein the peptide fragment is derived from the C-terminal acidic tail of α-synuclein, or its derivative, is responsible for resistance to environmental stresses and that a fusion protein comprising the peptide and a protein linked thereto is resistant to environmental stresses while maintaining the intrinsic properties of the protein.

### SUMMARY OF THE INVENTION

It is an object of the present invention to provide a peptide conferring resistance to environmental stresses, comprising a peptide fragment containing a sequence composed of 10 or more consecutive amino acid residues including five or more acidic amino acid residues, wherein the peptide fragment is derived from the C-terminal acidic tail of α-synuclein, or its derivative.

It is another object of the present invention to provide a fusion protein comprising said peptide and a fusion partner protein which is linked to the peptide, wherein the fusion protein is resistant to environmental stresses.

It is a further object of the present invention to provide a method of conferring resistance to environmental stresses to a protein of interest, comprising linking the protein to said peptide.

### BRIEF DESCRIPTION OF THE DRAWINGS

The above and other objects, features and other advantages of the present invention will be more clearly understood from the following detailed description taken in conjunction with the accompanying drawings, in which:

FIG. 1a is a schematic diagram of α-synuclein composed of the N-terminal amphipathic region (residues 1-60), the hydrophobic NAC region (residues 61-95) and the C-terminal acidic tail (residues 96-140);

FIG. 1b is a schematic diagram of fusion proteins GST-Syn1-140, GST-Syn1-60, GST-Syn61-95, GST-Syn61-140 and GST-Syn96-140, which are formed by linking peptides of full length α-synuclein, the amphipathic region, the NAC region, the NAC region and acidic tail regions, and the acidic tail region, respectively, to the C-terminus of glutathion S-transferase (GST), a heat-labile protein;

FIG. 2 is the results of SDS-polyacrylamide gel eletrophoresis (SDS-PAGE) showing thermal behaviors of α-synuclein and the GST protein (Lane 1: α-synuclein without heat treatment, Lane 2: GST without heat treatment, Lane 3: α-synuclein with heat treatment, Lane 4: GST with heat treatment);

FIG. 3 is the results of SDS-PAGE showing thermal behaviors of α-synuclein deletion mutants, prepared by treating the GST-α-synuclein fusion proteins with thrombin;

FIG. 4a is the results of SDS-PAGE showing thermal behaviors of GST-α-synuclein fusion proteins before (left panel) and after (right panel) boiling (Lane 1: GST-Syn1-140, Lane 2: GST-Syn1-60, Lane 3: GST-Syn61-95, Lane 4: GST-Syn61-140, Lane 5: GST-Syn96-140);

FIG. 4b is a graph of absorbance showing heat-induced aggregation of the GST-α-synuclein fusion proteins;

FIG. 5a is a graph of absorbance showing the effect of divalent cations on the heat-induced aggregation of GST-Syn1-140;

FIG. 5b is a graph of absorbance showing the effect of divalent cations on the heat-induced aggregation of GST-Syn61-140 and GST-Syn96-140;

FIG. 6a is a graph of absorbance for comparison of GST activities of GST and the GST-synuclein fusion proteins before and after heat treatment (▪: before heat treatment, □: after heat treatment);

FIG. 6b is a graph of absorbance showing enzyme activity (Left) and aggregation profile (Right) of GST and the GST-Syn96-140 according to temperature (-•-: GST, -◯-: GST-Syn96-140, bars indicating the standard deviation);

FIG. 7a is a graph showing far-UV CD spectrum and the melting curve of GST (the inset graph presenting the mean molar ellipticity per residue of the GST protein at 222 nm according to temperature);

FIG. 7b is a graph showing far-UV CD spectrum and the melting curve (inset graph) of GST-Syn96-140 (solid line: measurement at 25° C., dotted line: measurement at 100° C., dashed line: measurement after cooling from 100° C. to 25° C.);

FIG. 8a is a graph showing pH-induced aggregation of GST and GST-Syn96-140;

FIG. 8b is a graph showing metal-induced aggregation of GST and GST-Syn96-140;

FIG. 9 is the results of SDS-PAGE showing thermal behaviors of DHFR (dihydrofolate reductase) and the DHFR-Syn96-140 fusion protein before heat treatment and after heat treatment at 65° C. and 100° C., respectively, for 10 minutes (the last lane is a size marker protein);

FIG. 10a is a schematic diagram of the GST-synuclein fusion protein composed of GST and a fragment of the C-terminal acidic tail region of α-synuclein;

FIG. 10b is the results of SDS-PAGE showing thermal behaviors of the GST-(ATSαfragment) fusion protein containing peptides derived from the ATSα of concentration of 0.6 mg/ml before (the upper panel) and after (lower panel) boiling;

FIG. 10c is a graph of absorbance showing aggregation of GST and the GST-(ATSαfragment) fusion proteins induced by heat treatment at 65° C., wherein the GST-(ATSαfragment) fusion protein is in a concentration of 0.2 mg/ml (1: GST, 2: GST-Syn103-115, 3: GST-Syn114-126, 4: GST-Syn130-140, 5: GST-Syn119-140);

FIG. 10d is a graph of absorbance showing aggregation of GST and the GST-(ATSαfragment) fusion proteins induced by heat treatment at 80° C. for 10 minutes at a concentration in the range of 0.2 mg/ml to 1.0 mg/ml (1: GST, 2: GST-Syn103-115, 3: GST-Syn114-126, 4: GST-Syn130-140, 5: GST-Syn119-140, 6: GST-Syn96-140), respectively,

FIG. 11a is a schematic diagram of the GST-synuclein fusion proteins containing the C-terminal acidic tail region of α-synuclein (ATSα), β-synuclein (ATSβ) and γ-synuclein (ATSγ), respectively;

FIG. 11b is the results of SDS-PAGE showing thermal behaviors of the GST-ATS fusion proteins (GST-ATSα, GST-ATSβ and GST-ATSγ) after boiling for 10 minutes at the concentration of 0.6 mg/ml;

FIG. 11c is a graph of absorbance showing aggregation of GST and the GST-ATS fusion proteins induced by heat treatment at 65° C. at the concentration of 0.2 mg/ml (1: GST, 2: GST-ASTα, 3: GST-ATSβ, 4: GST-ATSγ);

FIG. 11d is a graph of absorbance showing aggregation of GST and the GST-ATS fusion proteins induced by heat treatment at 80° C. for 10 minutes at a concentration in the range of 0.2 mg/ml to 1.0 mg/ml (1: GST, 2: GST-ASTα, 3: GST-ATSβ, 4: GST-ATSγ);

FIG. 12a is a schematic diagram of the GST-polyglutamate fusion proteins (GST-E5 and GST-E10) containing the polyglutamate tail;

FIG. 12b is the results of SDS-PAGE analysis of the purified GST-E5 and GST-E10 fusion proteins;

FIG. 12c is a graph of absorbance showing aggregation of GST and GST-E5 and GST-E10 fusion proteins induced by heat treatment at 65° C. at the concentration of 0.2 mg/ml (1: GST, 2: GST-E5, 3: GST-E10);

FIG. 12d is a graph of absorbance showing aggregation of GST and GST-E5 and GST-E10 fusion proteins induced by heat treatment at 80° C. for 10 minutes at a concentration in the range of 0.2 mg/ml to 1.0 mg/ml (1: GST, 2: GST-E5, 3: GST-E10).

FIG. 13 is a schematic diagram of hGH and the hGH Syn119-140-hGH fusion proteins in which a Syn119-140 is fused to the N-terminus of hGH and to the C-terminus of hGH, respectively.

FIG. 14 is a photograph showing an SDS-PAGE result of purified hGH and Syn119-140-fused hGH proteins (lane 1: hGH, lane 2: ATS-hGH, lane 3: hGH-ATS).

FIG. 15 is a graph showing far-UV CD spectra of hGH, Syn119-140-hGH and hGH-Syn119-140 (solid line: hGH, dotted line: Syn119-140-hGH, dashed line: hGH-119-140).

FIG. 16 is a graph showing proliferation patterns of Nb2 cells in accordance with the concentrations of hGH, Syn119-140-hGH, and hGH-Syn119-140(◯: hGH, ▾: Syn119-140-hGH, ▴: hGH-Syn119-140, bars representing standard deviations)

FIG. 17 is a photograph showing a result of a Western blotting assay using an antibody against a tyrosine phosphorate form of STAT-5 (lanes 1, 5: control, lanes 2, 6: hGH, lanes 3, 6: Syn119-140-hGH, and lanes 4, 8: hGH-Syn119-140).

FIG. 18 is a graph showing the shaking-induced aggregation of hGH, Syn119-140-hGH and hGH-Syn119-140, in which absorbance is plotted over shaking time (•: hGH, ◯: Syn119-140-hGH, ▾: hGH-Syn119-140).

FIG. 19 is a photograph showing the aggregation behaviors of hGH, Syn119-140-hGH and hGH-Syn119-140 after shaking of 90 hours.

FIG. 20 shows HPLC gel filtration chromatograms of hGH, Syn119-140-hGH and hGH-Syn119-140 detected after and before shaking (solid line: before shaking, dotted line: after shaking of 90 hours).

FIG. 21 is a bar graph showing the repeated freezing/thawing-induced aggregation of hGH, Syn119-140-hGH and hGH-Syn119-140 in absorbance measurements in accordance with the freezing/thawing cycles (dark bar: hGH, white bar: Syn119-140-hGH, gray bar: hGH-Syn119-140).

FIG. 22 shows HPLC gel filtration chromatograms of hGH, Syn119-140-hGH and hGH-Syn119-140 detected before and after freezing/thawing (1: control before freezing/thawing, 2: after 5 freezing/thawing cycles, 3: after 10 freezing/thawing cycles, 4: after 15 freezing/thawing cycles).

FIG. 23 is a bar graph showing the pH-induced aggregation of hGH, Syn119-140-hGH and hGH-Syn119-140 in absorbance measurements (dark bar: hGH, white bar: Syn119-140-hGH, gray bar: hGH-Syn119-140).

FIG. 24 is a bar graph showing the aggregation of hGH, Syn119-140-hGH and hGH-Syn119-140 after storage at 25° C. and 37° C. in absorbance measurements (dark bar: 37° C., white bar: 25° C.).

FIG. 25 is a photograph showing an SDS-PAGE result of hGH, Syn119-140-hGH and hGH-Syn119-140 before and after storage for 30 days at 25° C. and 37° C. (lanes 1, 4, 6: hGH, lanes 2, 5, 7: Syn119-140-hGH, lanes 3, 6, 9: hGH-Syn119-140)

FIG. 26 is a bar graph showing the aggregation of hGH, Syn119-140-hGH and hGH-Syn119-140 after storage at 60° C. for 3 days in absorbance measurements.

FIG. 27 is a photograph showing an SDS-PAGE result of hGH, Syn119-140-hGH and hGH-Syn119-140 before and after storage at 60° C. for 3 days (lanes 1, 4: hGH, lanes 2, 5: A Syn119-140-hGH, lanes 3, 6: hGH-Syn119-140).

FIG. 28 is a photograph showing an SDS-PAGE result of hGH, Syn119-140-hGH and hGH-Syn119-140 after heat treatment at 80° C. and 100° C. for 10 min (lanes 1, 4, 6: hGH, lanes 2, 5, 7: Syn119-140-hGH, lanes 3, 6, 9: hGH-Syn119-140).

FIG. 29 is a graph showing the aggregation of hGH, Syn119-140-hGH and hGH-Syn119-140 induced by treatment at 80° C., in which absorbance at 405 nm is plotted over time (•: hGH, ◯: Syn119-140-hGH, ▾: hGH-Syn119-140).

FIG. 30 shows HPLC gel filtration chromatograms of hGH, Syn119-140-hGH and hGH-Syn119-140 detected after treatment for 10 min at various temperatures (1: 25° C., 2: 65° C., 3: 70° C., 4: 75° C., 5: 80° C.).

FIG. 31 is a graph showing the thermal denaturation of hGH, Syn119-140-hGH and hGH-Syn119-140 in CD spectra expressed in terms of the mean residue ellipticity at 222 nm over temperature (solid line: hGH, dotted line: Syn119-140-hGH, dashed line: hGH-Syn119-140);

FIG. 32 is a bar graph showing biological activities of the hGH proteins obtained from the supernatants after heat treatment at various temperatures (dark bar: hGH, white bar: Syn119-140-hGH, gray bar: hGH-Syn119-140)

FIG. 33 is a graph showing the pharmacokinetics of hGH, Syn119-140-hGH and hGH-Syn119-140, in which protein concentration is plotted over time.

FIG. 34a is a photograph showing an SDS-PAGE result of purified hGH, hGH-Syn119-140, and two fusion proteins hGH-ATSw containing a whole Syn119-140 peptide and hGH-ATSp containing fragment of Syn119-140 peptide.

FIG. 34b is a graph showing the heat-induced aggregation of hGH, hGH-Syn119-140, hGH-ATSw and hGH-ATSp in absorbance measurements after treatment at 100° C.

FIG. 34C is a graph showing the shaking-induced aggregation of hGH, hGH-Syn119-140, hGH-ATSw, and hGH-ATSp in absorbance measurements.

FIG. 34d is a graph showing the repeated freezing/thawing-induced aggregation of hGH, hGH-Syn119-140, hGH-ATSw, and hGH-ATSp in absorbance measurements.

FIG. 35a is a photograph showing an SDS-PAGE result of purified hGH, hGH-Syn119-140, and the hGH-Syn119-140proteins containing point mutant residues in the ATS region.

FIG. 35b is a graph showing the heat-induced aggregation of hGH, hGH-Syn119-140, and the hGH-Syn119-140 proteins containing point mutant residues in the ATS region in absorbance measurements after treatment at 100° C.

FIG. 35C is a graph showing the shaking-induced aggregation of hGH, hGH-Syn119-140, and the hGH-Syn119-140 proteins containing point mutant residues in the ATS region in absorbance measurements.

FIG. 35d is a graph showing the repeated freezing/thawing-induced aggregation of hGH, hGH-Syn119-140, and the hGH-Syn119-140 proteins containing point mutant residues in the ATS region in absorbance measurements.

FIG. 36a is a photograph showing an SDS-PAGE result of purified hGH, hGH-Syn119-140, hGH-Synβ113-134 and hGH-Synγ106-127.

FIG. 36b is a graph showing the heat-induced aggregation of hGH, hGH-Syn119-140, hGH-Synβ113-134 and hGH-Synγ106-127 in absorbance measurements after heat treatment.

FIG. 36C is a graph showing the shaking-induced aggregation of hGH, hGH-Syn119-140, hGH-Synβ113-134 and hGH-Synγ106-127 in absorbance measurements.

FIG. 36d is a graph showing the repeated freezing/thawing-induced aggregation of hGH, hGH-Syn119-140, hGH-Synβ113-134 and hGH-Synγ106-127 in absorbance measurements.

FIG. 37a is a photograph showing an SDS-PAGE result of purified GCSF and GCSF-Syn119-140.

FIG. 37b is a graph showing the heat-induced aggregation of GCSF and GCSF-Syn119-140 in absorbance measurements after heat treatment at 40-60° C.

FIG. 37C is a graph showing the shaking-induced aggregation of GCSF and GCSF-Syn119-140

FIG. 37d is a graph showing the repeated freezing/thawing-induced aggregation of GCSF and GCSF-Syn119-140 in absorbance measurements.

FIG. 38a is a photograph showing an SDS-PAGE result of purified hLeptin and hLeptin-Syn119-140.

FIG. 38b is a graph showing the heat-induced aggregation of hLeptin and hLeptin-Syn119-140 in absorbance measurements after heat treatment at 40-70° C.

FIG. 38C is a graph showing the shaking-induced aggregation of hLeptin and hLeptin-Syn119-140 in absorbance measurements.

FIG. 38d is a graph showing the repeated freezing/thawing-induced aggregation of hLeptin and hLeptin-Syn119-140 in absorbance measurements.

### DISCLOSURE OF THE INVENTION

In one embodiment, the present invention is concerned with a peptide conferring environmental stress resistance, a peptide fragment containing a sequence composed of 10 or more consecutive amino acid residues including five or more acidic amino acid residues, wherein the peptide fragment is derived from the C-terminal acidic tail of synuclein, or its derivative.

In more detail, the present invention relate to a peptide conferring resistance to environmental stress, comprising (i) a peptide fragment containing a sequence composed of 10 or more consecutive amino acid residues including five or more acidic amino acid residues, wherein the peptide fragment is derived from SEQ ID NO:1 corresponding to amino acid residues 96-140 of the C-terminal acidic tail of α-synuclein, or its derivative, (ii) a peptide fragment containing a sequence composed of 10 or more consecutive amino acid residues including five or more acidic amino acid residues, wherein the peptide fragment is derived from SEQ ID NO:2 corresponding to amino acid residues 85-134 of the C-terminal acidic tail of β-synuclein, or its derivative, (iii) a peptide fragment containing a sequence composed of 10 or more consecutive amino acid residues including five or more acidic amino acid residues, wherein the peptide fragment is derived from SEQ ID NO:3 corresponding to amino acid residues 96-127 of the C-terminal acidic tail of γ-synuclein, or its derivative, or (iv) a peptide fragment containing a sequence composed of 10 or more consecutive amino acid residues including five or more acidic amino acid residues, wherein the peptide fragment is derived from SEQ ID NO:4 corresponding to amino acid residues 96-127 of the C-terminal acidic tail of synoretin, or its derivative.

The term “environmental stresses”, as used herein, refers to physical or chemical actions which may cause denaturation of natural or non-natural proteins. In connection with this, the “denaturation of protein” means that a high order structure of a protein is irreversibly changed by physical actions such as heating, freezing and drying, or chemical actions such as acids, alkalis, metal ions, oxidizing/reducing agent or organic solvents, generally including phenomena accompanying with loss of biological functions, reduction in solubility, decrease or increase in reactivity, ease of decomposition by enzyme, loss of crystallinity, change of physicochemical properties, modified blue shift, etc. Examples of the environmental stresses which may denature the proteins in the present invention include physical factors such as temperature, moisture, pH, electrolyte, reduced sugar, pressurizing, drying, freezing, interfacial tension, light beam, repetitive freezing and thawing, hyperconcentration, and chemical factors such as acids, alkalis, neutralized salts, organic solvents, metal ions, oxidizing/reducing agents, etc.

Specifically, the environmental stresses according to the present invention include temperature, moisture, pH, metal ions, electrolytes and reduced sugars which may denature proteins. Most proteins begin to denature at a temperature between 60 to 70° C. and the denaturation rate increases as the temperature rises. For example, when the temperature rises by 10° C., the denaturation rates of albumin and hemoglobin increase 20 times and 13 times, respectively. However, when the temperature is sharply raised, the aggregation temperature may go up. When proteins thermally denature, water is needed. Water helps movement of polypeptide chains upon denaturing or refolding. Thus, if water is sufficient, the thermal denaturation may take place at a lower temperature. The thermal denaturation of proteins is also associated with pH and generally, at an acidic pH near pI the denaturation occurs faster. Using such property, when cooking fish, a small amount of vinegal is added to rapidly harden the fish fresh. Further, the denaturation of proteins may be induced by addition of electrolytes (salts). Upon addition of the electrolyte, cations in the electrolyte including salt compounds and sulfates may neutralize negative charges of a protein, rendering pH to be pI. If reduced sugar is present when applying heat to a protein, Maillard reaction, non-enzymatic browning, occurs to destroy essential amino acids.

Among the environmental stresses according to the present invention, are included pressurizing and dry circumstances which may cause denaturation of proteins. In general, proteins are denatured by application of a high pressure in the range of 5000 to 10000 atm or by sonication. Particularly, soluble proteins may be denatured by drying. As drying progresses, moisture existing between polypeptide chains disappears, upon which adjacent peptide chains are recombined to form a more solid structure.

Among the environmental stresses according to the present invention, are included freezing circumstances which may cause denaturation of proteins. For example, when is meat frozen, water is first crystallized as ice crystals because of its weak bonding force. Consequently, salt concentration in the remaining liquid is increased, causing salting out, by which proteins are denatured. Protein denaturation is aggravated as freezing and thawing are repeated. Among another environmental stresses, interfacial tension is included. Proteins are denatured upon spreading as a single molecular layer on the interface, resulting in aggregation. Further, among another environmental stresses, irradiation of light which may cause denaturation of proteins is included. Upon irradiation of light to protein, bondings in the protein tertiary structure are broken, resulting in denaturation. Acids, alkalis, neutral salts, organic solvents such as alcohols and acetones, and metal ions are included among the environmental stresses for the purpose of the present invention. When acid, alkali is added to protein solutions, (+) and (−) charges are changed, which in turn causes alteration of ionic bonds, which are intimately connected with the high order structure, thereby resulting in denaturation of proteins.

The C-terminal acidic tails of synuclein family show resistance to these environmental stresses.

The term “synuclein family”, as used herein, refers to a group of unstructured soluble proteins chiefly expressed in the nervous tissues and found in fish or higher organisms such as humans, mice, birds, oxen, etc. (Clayton and George, Trends in Neuroscience 21, 249-254 (1998)). As members of the synuclein family, α-synuclein, β-synuclein, γ-synuclein and synoretin are known. α- and β-synuclein are enriched in the brain tissue, especially in presynaptic terminals and γ-synuclein in the peripheral nervous system. α- and β-synucleins are believed to share functional homology with each other because they are very similar in amino acid sequences and protein distribution. Synoretin, sharing high homology with γ-synuclein, is enriched in the retina.

The synuclein family has a structural characteristic of three independent domains consisting of an amino-terminal amphiphilic region, a hydrophobic NAC region, and a carboxy-terminal acidic tail. The N-terminal amphipathic region of the synuclein family members is strictly conserved among the synuclein family members from the Torpedo to humans, but the C-terminal acidic tails are very diverse in size as well as in sequence (Lucking C. B. and Brice A., Cell Mol. Life Sci., 57, 1894-1908 (2000); Iwai A. Biochem. Biophys. Acta 1502, 95-109 (2000); Hashimoto M. and Masliah E. Brain Pathol., 9, 707-720 (1999); Lavedan C. Genome Res. 8, 871-880 (1998)).

More specifically, “the C-terminal acidic tail of α-synuclein (amino acid residues 96-140)” is abbreviated to “ATSα”, “Synα96-140” or “Syn96-140”; “the C-terminal acidic tail of β-synuclein (amino acid residues 85-134)” is abbreviated to “ATSβ” or Synβ85-134; “the C-terminal acidic tail of γ-synuclein (amino acid residues 96-127)” is abbreviated to “ATSγ” or Synγ96-127.

Although synuclein has to be further proven in function, some research identified α- and β-synucleins as inhibitors of mammalian phospholipase D2 (Jenco et al., Biochemistry 37, 4901-4909 (1998)) and revealed that tsynuclein alters the metabolism of the neuronal cytoskeleton (Buchman et al., Nat. Neurosci. 1, 101-103 (1998)).

Particularly, α-synuclein has the following characteristics. Since α-synuclein is intrinsically unstructured in its native state, it may interact with many other proteins or ligands (Kim J., Molecules and Cells, 7, 78-83 (1997); Weinreb P. H. et al., Biochemistry, 35, 13709-13715 (1996)). α-synuclein acquires an increased level of secondary structure, when it associates with small acidic phospholipid vesicles, detergents, organic solvents and some metal ions (Eliezer D. et al., J. Mol. Biol., 307, 1061-1073 (2001); Kim T. D. et al., Protein Science, 9, 2489-2496 (2000); Davidson W. S. et al., J. Biol. Chem., 273-9443-9 (1998); Weinreb P. H. et al., Biochemistry, 35, 13709-13715 (1996); Paik S. R. et al., Biochem. J., 340, 821-8 (1999)). As mentioned above, α-synuclein is extremely heat resistant, which is possibly due to the abnormal primary and tertiary structure features.

In addition, α-synuclein has a chaperone-like function against thermal and chemical stress as demonstrated by the suppression of the aggregation of chemically or thermally denatured proteins in the presence of α-synuclein (Thomas et al., protein science, 9, 2489-2496 (2000); Jose M et al., FEBS Letter, 474, 116-119 (2000)). However, the independent function and activity of each region of α-synuclein remains to be researched. Particularly, before the study of the present inventors it was not revealed that ATSα alone is resistant to environmental stresses.

In the U.S. Pat. No. 6,858,704 which is included in this application as a reference as a whole, the inventor disclosed that Synuclein-derived peptides, more particularly, peptides derived from ATSα of SEQ ID NO:5, SEQ ID NO:6, SEQ ID NO:7, and SEQ ID NO:8, peptide of ATSβ of SEQ ID NO:2, peptide of ATSγ of SEQ ID NO:3, and peptide of C-terminal amino acidic tail of synoretin of SEQ ID NO: 4 are resistant to the environmental stresses.

The significance of the invention of the above US patent resides in the finding that each ATS region alone has the same resistance to environmental stresses as that of an intact synuclein. Each of environmental stress-resistant ATS regions has advantages over the intact synuclein in an aspect of industrial applicability because synuclein, when administered repetitively or in excess doses, may have unexpectably serious side effects or toxicity as it maintains its intrinsic activity. Further, the absence of the information on the accurate in-vivo activity of synuclein makes it impossible to expect accurate side effects of synuclein and provide a counterplan thereagainst. When synuclein is deprived of the amino terminal amphipathic region and hydrophobic NAC region, both strictly conserved among the synuclein family members, the remaining fragment, that is, ATS alone loses the intrinsic activity of synuclein, but retains the environmental stress resistance.

Further study was made to the characteristics on the amino acid sequence by which, although the C-terminal region is very diverse in size and amino acid sequence compared to the N-terminal amphipathic region and the hydrophobic NAC region, both well conserved among the synuclein family members, all the ATS regions are resistant to environmental stresses. For the purpose of the invention, the inventors have studied the relations between the activities resistant to the environmental stress and the length of the ATS derived peptides.

In the present application, the present inventor has found that an ATSα-derived peptide fragment and its derivatives show environmental stress resistance. It is also revealed that, in addition to ATSα, each of ATSβ and ATSγ is resistant to environmental stresses.

Fusion proteins containing peptide fragments of various lengths corresponding to various points of ATSα were prepared. As fusion proteins in which ATSα fragments bind to GST, GST-Syn103-115 containing the amino acid residues 103-115 (SEQ ID NO:5) corresponding to a front portion of ATSα, GST-Syn114-126 containing the amino acid residues 114-126 (SEQ ID NO:6) corresponding to a medium portion of ATSα, GST-Syn119-140 containing the amino acid residues 119-140 (SEQ ID NO:7) corresponding to a medium and terminal portion of ATSα, and GST-Syn130-140 containing the amino acid residues 130-140 (SEQ ID NO:8) corresponding to a terminal portion of ATSα were constructed. When treated at 100° C. for 10 min, GAS-Syn96-140 containing 45 amino acid residues corresponding to the full length ASTα and GST-Syn119-140 containing 22 amino acid residues did not precipitate at all while precipitation was observed in GST-Syn103-115, GST-Syn114-126 and GST-Syn130-140, each of which contains 11-13 amino acid residues. When treated at 65° C. for 2 min, most GST proteins aggregated, and heat treatment at 65° C. for 3 min caused aggregation in all GST proteins, but not in the GST-ASTαfusion constructs at all. The results, obtained from the experiment in which the GST-ATSαconstructs having various concentrations from 0.2 mg/ml to 1.0 mg/ml were treated at 80° C. for 10 min, exhibited that GST-Syn96-140 containing the full length ATSα and GST-Syn119-140 containing 22 amino acid resides of ATSα did not precipitate at all irrespective of concentration whereas GST-Syn103-115, GST-Syn114-126 and GST-Syn130-140, each containing 11-13 amino acid residues of ATSα, increasingly aggregated as the concentration increased. This demonstrates that the environmental stress resistance of ATS fragments increases with the peptide lengths thereof irrespective of the position at which the fragments are cut.

Using fragments shorter and longer than Syn119-140, an examination was made of the effect of peptide length on the environmental stress resistance. As fusion proteins of hGH and GST, hGH-Syn113-140 containing the amino acid residues 113-140 (SEQ ID NO:9) of ATSα, and GST-Syn119-135 containing the amino acid residues 119-135 (SEQ ID NO:10) of ATSα were prepared. Then the two fusion proteins of hGH-Syn113-140 and GST-Syn119-135 were compared with hGH-Syn119-140 containing the amino acid residues 119-140 of ATSα with respect to the resistance to the environmental stresses such as heat treatment, stirring, repeated freezing and thawing, etc.

From the results of the examination, it is apparent that the fusion proteins, hGH-Syn119-140, hGH-Syn-113-140, and hGH-Syn119-135, containing the synuclein-derived peptide are more stable than the natural Growth hormone. Upon stirring, hGH-ATSw containing the longer synuclein-derived peptide is slightly more stable than hGH-ATSp containing the shorter synuclein-derived peptide. Consequently, it is apparent that the resistance to environmental stresses of the fusion protein is highly dependent on the characteristics of the unique amino acid sequence and length of the peptide derived from ATS. The longer the length of the ATS-derived peptide is, the more stable is the ATS-derived peptide containing fusion protein. Not only ASTα-derived peptide fragment, but also ATSβ- and ATSγ-derived peptide fragment have the resistance to the environmental stresses.

The ATS region features redundant negatively charged, acidic amino acid residues. GST-polyglutamate fusion proteins containing the acidic tails consisting of polyglutamate were examined for heat resistance. Both GST-E5 and GST-E10, which contain fusion peptides consisting of five and ten glutamate residues, respectively, aggregated after heat treatment at 100° C. Heat treatment at 65° C. for 2-3 min aggregated GST alone completely and aggregated GST-E5 to a significant extent, but did not aggregate GST-E10 at all. When proteins were treated at 80° C. for 10 min with a gradual concentration change from 0.2 mg/ml to 1.0 mg/ml, aggregation was found in GST-E10 to a lesser extent than in GST, and GST-E5 aggregated only a little. This shows that the more negative charges there are, the higher the environmental stress resistance is.

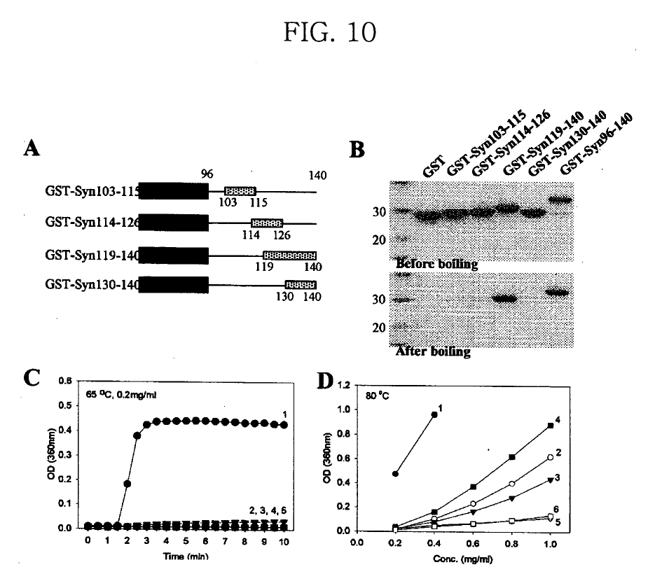

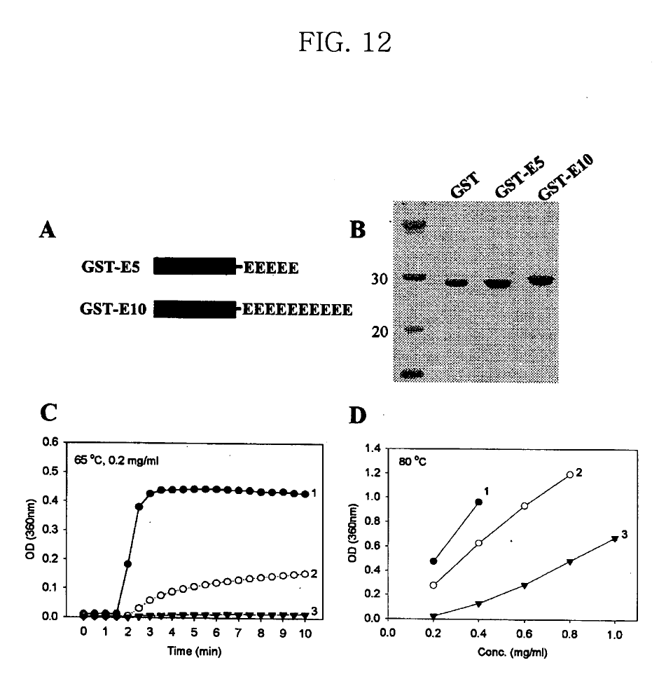

Additionally, GST-Syn130-140 is found to show heat resistance far greater than that of GST-E5 which contains the same number of negative charges and a little lower than that of GST-E10 which contains twice the number of negative charges (see FIGS. 10d and 12d). This result suggests that both the number of negatively charged amino acid residues in ATS and the peptide fragment length of ATS act as important factors conferring resistance to environmental stresses to the fusion proteins.

Therefore, it is understood that the characteristic amino acid sequence of ATS, which contains many acidic amino acid residues, and the length of the peptide fragment of the ATS plays an important role in conferring resistance to environmental stresses.

In order to provide a fusion protein that is resistant to environmental stress according to invention, it is of advantageous to be able to select various ATS-derived peptides. In this regard, since the smallest activity unit of the Synuclein C-terminal acidic tail derived peptides has been identified in the present application, it is very advantageous to select a suitable peptide to be linked to a protein that is desired to enhance resistance to environmental stress in accordance with the protein.

According to the present invention, the requirement for acquiring resistance to environmental stresses is that an ATS-derived peptide fragment contains at least five acidic amino acid residues in addition to being at least 10 amino acid residues long. Based on this finding of the present invention, those skilled in the art can derive various environmental stress-resistant peptide fragments from the ATS region.

The ATS of the present invention may originate from various animals including cattle, goats, pigs, mice, rabbits, hamsters, rats, guinea pigs, etc., with preference for human origin. The ATS of the present invention may be the C-terminal acidic tail region of any member of the synuclein family, and preferably the C-terminal acidic tail region of α-, β-, γ-synuclein or synoretin.

The C-terminal acidic tail region of human α-synuclein, identified as SEQ ID NO: 1, corresponds to residues 96-140, the C-terminal acidic tail region of human β-synuclein, identified as SEQ ID NO:2, to residues 85-134, the C-terminal acidic tail region of human tsynuclein, identified as SEQ ID NO:3, to residues 96-127, and the acidic tail region of human synoretin, identified as SEQ ID NO:4, to residues 96-127.

A peptide conferring resistance to environmental stress is preferably, comprising a peptide fragment containing a sequence composed of 10 or more consecutive amino acid residues including five or more acidic amino acid residues, wherein the peptide fragment is derived from SEQ ID NO:1 corresponding to amino acid residues 96-140 of the C-terminal acidic tail of α-synuclein, a peptide fragment containing a sequence composed of 10 or more consecutive amino acid residues including five or more acidic amino acid residues, wherein the peptide fragment is derived from SEQ ID NO:2 corresponding to amino acid residues 85-134 of the C-terminal acidic tail of β-synuclein, a peptide fragment containing a sequence composed of 10 or more consecutive amino acid residues including five or more acidic amino acid residues, wherein the peptide fragment is derived from SEQ ID NO:3 corresponding to amino acid residues 96-127 of the C-terminal acidic tail of γ-synuclein, and a peptide fragment containing a sequence composed of 10 or more consecutive amino acid residues including five or more acidic amino acid residues, wherein the peptide fragment is derived from SEQ ID NO:4 corresponding to amino acid residues 96-127 of the C-terminal acidic tail of synoretin.

A peptide of SEQ ID NO:5, SEQ ID NO:6, SEQ ID NO:7, SEQ ID NO:8, SEQ ID NO:9, SEQ ID NO:17, or SEQ ID NO: 18, which confers resistance to environmental stress, is preferable.

Not only the peptides having the wild type amino acid sequences, but also the derivatives thereof, are considered to be the environmental stress-resistant peptides according to the present invention.

The term “peptide mutants” or “peptide derivatives” as used herein refers to peptides, occurring naturally or artificially, which are different in amino acid sequence from wild-type peptides due to the deletion, insertion, non-conservative substitution or conservative substitution of amino acids, or combinations thereof. As long as peptide mutants are resistant to environmental stresses, they are within the scope of the present invention.

Based on the structure of a protein, a suitable mutant of C-terminal acidic tail of Synuclein can be prepared. Since the unique characteristics of the amino acid sequence of C-terminal acidic tail of Synuclein that is resistant to the environmental stress has been revealed in the application, persons skilled in this particular field can appreciate that various derivatives being resistant to environmental stress can easily be made. Mutants may be the equivalents having the same activity as the wild type peptide or be a peptide having more activity than the wild type peptide.

One or more amino acid residues in C-terminal acidic tail of α-Synuclein, C-terminal acidic tail of β-Synuclein, C-terminal acidic tail of γ-Synuclein, and C-terminal acidic tail of synoretin can be substituted with another amino acid residue that differs from the original amino acid residue in the peptide. The position at which the amino acid is substituted is not limited.

In a preferred embodiment, the peptide fragment derivative of the C-terminal acidic tail of α-Synuclein is selected from the group consisting of the mutants of which one or more amino acid residues at residue numbers 122, 123, 124, 127, 133 and 140 are substituted with another amino acid residue that differs from the original amino acid residues of the C-terminal acidic tail of α-Synuclein.

In a specific embodiment of the invention, mutants of E123A (SEQ ID NO:11), Y133A (SEQ ID NO:12), A124E (SEQ ID NO: 13), N122V (SEQ ID NO:14), M127S (SEQ ID NO:15) and A140S (SEQ ID NO:16) of Syn119-140 have been prepared. Each of them is as long as 22 amino acid residues, like Syn119-140, but has a mutated amino acid. Both E123A, which lacks one acidic amino acid residue, and A124E, which has one more acidic amino acid residue, show Syn119-140-like activity against environmental stresses such as heat, stirring, freezing/thawing, etc. Also, the mutants that have more hydrophobic residues (Y123A and N122V) show activity similar to that of Syn119-140. Further, similar activity is observed in mutants which are substitution-mutated at amino acid residues which are not conserved among the synuclein family.

Namely, although ATS peptides resistant to environmental stresses undergo substitution mutation at one or more amino acid residues, they do not lose their resistant activity regardless of the position to be mutated and the amino type to be substituted with. Therefore, as long as the mutants confer resistance to environmental stresses, they are within the scope of the present invention. Preferable are mutants which have enhanced functionality and/or stability due to the mutation of the amino acid sequence.

From the results of the above, it is understood that even though one or more amino acids are substituted with another amino acid in the peptide of the invention, as long as the mutant has the characteristics of the unique amino acid sequence of C-terminal acidic tail of Synuclein that has the resistance to environmental stress, the activity of the mutant is maintained as a peptide of the invention being resistant to the environmental stress. Accordingly, if a peptide comprises a sequence composed of 10 or more consecutive amino acid residues including five or more acidic amino acid residues, wherein the peptide fragment is derived from the amino acid residues of the C-terminal acidic tail of synuclein, mutants of said peptide in which one or more amino acid residues are substituted with another amino acid are also included in the scope of the present invention. It is more preferable that the activity and/or stability of the mutants of the peptide of the invention increase.

Mutants can be isolated from nature if it is naturally occurring, and can be synthesized (Merrifield, J. Amer. Chem. Soc., 85: 2149-2156, 1963) or be constructed by a recombinant DNA synthesizing method (Sambrook et al., Molecular Cloning, Cold Spring Harbour Laboratory Press, New York, USA, 2^{nd} ed., 1989). Preferably, gene recombinant techniques are used.

Inducing a mutagenesis in amino acid sequence of a wild type peptide can be performed by a mutagenesis in the nucleotide sequence encoding the wild type peptide. This method is well known to persons skilled in this particular field. In the present invention, site-directed mutagenesis was used.

In another embodiment, the present invention is concerned with a fusion protein comprising the peptide of ATS of the invention and a fusion partner protein which is linked to the peptide.

For the purpose of the present invention, “fusion protein” means any fusion protein prepared by fusing any of the ATS-derived peptides of the present invention to a fusion partner protein. No special limitations are imposed on the position at which a fusion partner protein is bonded to the peptide. An example of the fusion proteins is one polypeptide sequence containing a peptide according to the present invention linked to a fusion partner protein through a peptide bond, which can be readily obtained through translation from a recombinant gene prepared by gene manipulation. One or more peptides of the present invention may be linked to N-terminus, C-terminus or both termini of a fusion partner protein. Peptides linked to each of the N- and C-terminus of a fusion partner protein may be the same or different.

The type and the length of ATS peptide fragment linking to a fusion partner protein could be selected depending on the size and the property of fusion partner protein.

A linker may be interposed between a peptide of the present invention and a fusion partner protein. The linker, which plays a bridge role by connecting a peptide of the present invention to a fusion partner protein, may be a peptide or not. A peptide linker is a sequence consisting of 1-20 amino acid residues linked through a peptide bond, and preferable is an immunologically inactive one.

The “fusion partner protein” refers to any proteins which is desired to have increased resistance to environmental stresses, particularly, proteins which are environmental stress-labile in themselves. The term “environmental stress-labile proteins” refers to proteins that are easily denatured by environmental stresses. The “denaturation” means the same as defined above. The environmental stress-labile proteins are well-known according to the denaturing factors.

Any protein which needs to have enhanced resistance to environmental stresses may be used as a fusion partner protein without limitations. Commercially or medicinally useful proteins which need better resistance to environmental stresses are exemplified by various physiologically active polypeptides such as cytokines, interleukins, interleukin-associated proteins, enzymes, antibodies, growth factors, transcription factors, blood factors, vaccines, structural proteins, ligand proteins, ligand receptors, cell surface antibodies, receptor antagonists, etc., or their derivatives or analogs.

Concrete examples of the fusion partner proteins include glutathione S-transferase, dihydrofolate reductase, growth hormones, leptin, growth hormone-releasing peptides, interferons, interferon receptors, colony-stimulating factors, glucagon-like peptides (GLP-1, etc.), G-protein-coupled receptor, interleukins, interleukin receptors, interleukin-associated proteins, cytokine-associated proteins, macrophage-activating factors, macrophage peptides, B-cell factors, T-cell factors, protein A, suppressive factor of allergy, cell necrosis glycoprotein, immune toxins, lymphotoxins, tumor necrosis factors, tumor inhibitory factor, transforming growth factor, alpha-1 antitrypsin, albumin, alpha-lactalbimin, apolipoprotein-E, erythroprotein, hyper-glycosylated erythroprotein, angiopoietins, hemoglobin, thrombin, thrombin receptor activating peptide, thrombomodulin , factorVII, factor VIIa, factorVIII, factor IX, factor XIII, plasminogen activator, fibrin binding protein, urokinase, steptokinase, hirudin, protein C,C-reactive protein, renin inhibitor, collagenase inhibitor, superoxide dismutase, leptin, platelet derived growth hormone, epithelial growth factor, epidermal growth factor, angiostatin, angiotensin, osteogenic growth factor, osteogenesis stimulating protein, calcitonin, insulin, atriopeptin, cartilage inducing factor, elcatonin, connective tissue activator protein, tissue factor pathway inhibitor, follicle stimulating hormone, luteinizing hormone, luteinizing hormone-releasing hormone, nerve growth factor, parathyroid hormone, relaxin, secretin, somatomedin, insulin-like growth factor, adrenocorticotrophic hormone, glucagon, cholecystokinin, pancreatic polypeptide, gastrin releasing peptide, corticotropin releasing factor, thyroid stimulating hormone, autotaxin, lactoferrin, myostatin, receptors, receptor antagonists, cell surface antigens, virus-derived vaccine antigens, monoclonal antibodies, polyclonal antibodies, but are not limited thereto.

A preferred embodiment of this invention, GST-Syn96-140(SEQ ID NO:80), DHFR-Syn96-140(SEQ ID NO:81), GST-Syn103-115(SEQ ID NO:82), GST-Syn114-126(SEQ ID NO:83), GST-Syn119-140(SEQ ID NO:84), GST-Syn130-140(SEQ ID NO:85), GST-Synβ(SEQ ID NO:86), GST-Synγ(SEQ ID NO:87), hGH-syn119-140(SEQ ID NO:91), syn119-140-hGH(SEQ ID NO:93), hGH-syn113-140(SEQ ID NO:94), hGH-syn119-135(SEQ ID NO:95), hGH-synE123A(SEQ ID NO:96), hGH-synY133A(SEQ ID NO:97), hGH-synA124E(SEQ ID NO:98), hGH-synN122V(SEQ ID NO:99), hGH-synM127S(SEQ ID NO:100), hGH-synA140S(SEQ ID NO:101), hGH-synβ113-134(SEQ ID NO:102), hGH-synγ106-127(SEQ ID NO:103), GCSF-syn119-140(SEQ ID NO:104) and hLeptin-syn119-140(SEQ ID NO: 105) were prepare as a fusion protein, GST, DHFR, GH or leptin was linked to a peptide fragment of ATSα, ATSβ or ATSγ, or its derivative,.

A fusion protein of the present invention is characterized by that denaturation of the fusion partner protein is suppressed, through linkage to the peptide.

When being linked to a peptide according to the present invention, fusion partner protein, irrespective of type such as glutathione S-transferase (GST), dihydrofolate reductase (DHFR), growth hormone (GH), leptin, etc., is inhibited from being denatured against environmental stresses such as heat, stirring, repetitive freezing and thawing. Fusion partner proteins are found to retain higher blood levels in vivo as well as in vitro when bonded to the peptides of the present invention than when alone. These results suggest that the peptides of the present invention inhibit the denaturation of the partner proteins and thus stabilize them.

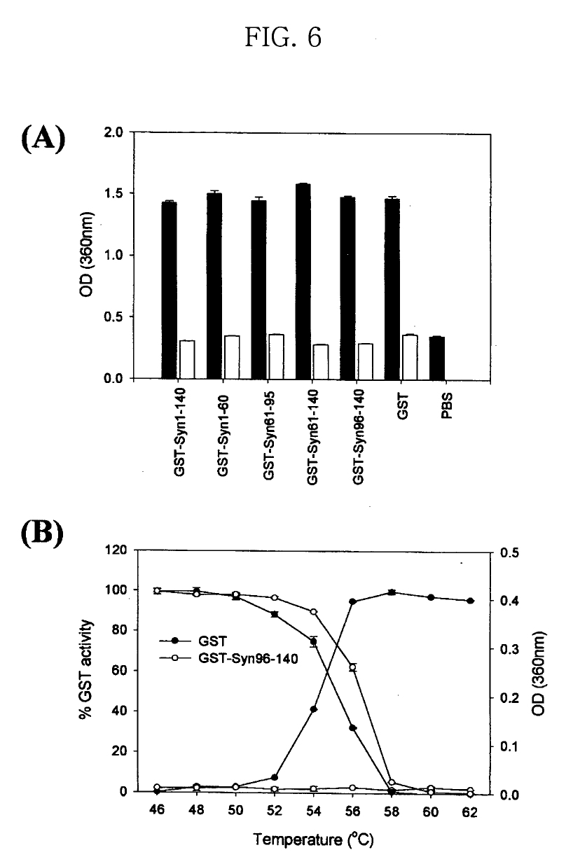

Also, a fusion protein of the present invention is characterized by that the activity of a partner protein is retained, although existing as being linked to the peptide of the present invention. Because the activity of physiologically functional proteins is determined by their structures, it sharply decreases in most of the proteins that are fused with other proteins. So that, the use of a protein in the form of a fusion protein, even if it is improved in stability when in the form of a fusion protein, is not efficient in practical in vivo availability due to an activity decrease. However, a protein of interest fused to the peptide of the present invention shows the same physiological activity as dose the protein alone (FIG. 6a).

Also, a fusion protein of the present invention is characterized by that solubility of fusion partner protein is increased, through linkage to the peptide. A fusion partner protein which is fused to the peptide of the present invention shows increased solubility. Redundant in negatively charged amino acids such as Glu or Asp, the amino acid sequences of ATS have very low pI values. As a rule, because the solubility of a protein is proportional to the square of its net charges (Tanford, 1961, in Physical Chemistry of macromolecules), an ATS fusion protein is expected to increase in solubility compared to the wild type. As demonstrated in the following examples, ATS fusion proteins are found to have far higher solubility than are their wild types. For example, fusion with an ATS peptide was found to increase solubility by 20% for GST, about two fold for hGH, and about five fold for leptin. Therefore, ATS-derived peptides can be used in concentrating proteins of interest as well as in enhancing their stability.

Also, the fusion partner protein, originally expressed to an inclusion body, can be expressed to a soluble form by linking the fusion partner protein to said peptide derived from the C-terminal acidic tail of synuclein. Also, refolding efficiency of fusion partner protein, originally expressed to the inclusion body, can increase by linking to the peptide. Accordingly, the fusion partner protein can be easily isolated and purified by linking the fusion partner protein to the peptide.

In a still further embodiment, the present invention is concerned with a method of conferring resistance to environmental stress to a protein of interest, comprising linking the protein to the peptide.

As it retains its intrinsic activity in addition to having increased resistance to environmental stresses, a fusion partner protein bonded to an ATS-derived peptide of the present invention shows higher in vivo availability in practice than when it exists alone.

Methods for the Preparation of a fusion protein in which a fusion partner protein is linked to a peptide of the present invention are not particularly restricted and may be based on, for example, genetic recombination by which two nucleotide sequences encoding a peptide of the present invention and a fusion partner protein, respectively, are digested with general restriction enzymes and ligated to each other to produce one nucleotide sequence which is then translated into the fusion protein.

In another embodiment, the present invention is concerned methods for preparing a peptide conferring resistance to environmental stress, comprising a peptide fragment containing a sequence composed of 10 or more consecutive amino acid residues including five or more acidic amino acid residues, wherein the peptide fragment is derived from the C-terminal acidic tail of α-synuclein, or its derivative.

The peptides of the present invention which are to be fused to target proteins can be easily prepared by chemical synthesis widely known to those skilled in the field of biochemistry (Creighton, Proteins: Structures and Molecular Principles, W. H. Freeman and Co., NY (1983)). Representative methods include liquid or solid phase synthesis, fragment condensation, F-MOC or T-BOC chemistry [Chemical Approaches to the Synthesis of Peptides and Proteins, Williams et al., Eds., CRC Press, Boca Raton Fla., (1997); A Practical Approach, Atherton & Sheppard, Eds., IRL Press, Oxford, England (1989)].

The peptides according to the present invention can be synthesized by performing the condensation reaction between protected amino acids by the conventional solid-phase method, beginning with the C-terminal and progressing sequentially with the first amino acid, the second amino acid, the third amino acid, and the like. After the condensation reaction, the protecting groups and the carrier connected with the C-terminal amino acid may be removed by a known method such as acid decomposition or aminolysis. The above-described peptide synthesis method is described in detail in literature [Gross and Meienhofer's, The peptides, vol 2., Academic Press (1980)].

The solid-phase carrier, which can be used in the synthesis of the peptides according to the present invention, includes polystyrene resins of substituted benzyl type, polystyrene resins of hydroxymethylphenylacetic amid form, substituted benzhydrylpolystyrene resins and polyacrylamide resins, having a functional group capable of bonding to peptides.

The protecting groups for initial protected amino acids are any protecting groups commonly used in peptide syntheses, including those readily removable by conventional methods such as acid decomposition, reduction or aminolysis. Specific examples of such amino protecting groups include formyl; trifluoroacetyl; benzyloxycarbonyl; substituted benzyloxycarbonyl such as (ortho- para-)chlorobenzyloxycarbonyl and (ortho- para-)bromobenzyloxycarbonyl; and aliphatic oxycarbonyl such as t-butoxycarbonyl and t-amiloxycarbonyl. The carboxyl groups of amino acids can be protected through conversion into ester groups. The ester groups include benzyl esters, substituted benzyl esters such as methoxybenzyl ester; alkyl esters such as cyclohexyl ester, cycloheptyl ester or t-butyl ester. The guanidino residue may be protected by nitro; or arylsulfonyl such as tosyl, methoxybenzensulfonyl or mesitylenesulfonyl, though it does not need a protecting group. The indole group of tryptophan may be protected by formyl or may not be protected.

Removal of protecting groups and carriers from peptides can be carried out using anhydrous hydrofluoride in the presence of various scavengers. Examples of the scavengers include those commonly used in peptide syntheses such as anisole, (ortho-, metha-, para-)cresol, dimethylsulfide, Co-cresol, ethanendiol and mercaptopyridine.

In other means, the peptides according to the present invention can be prepared by genetic engineering methods. Firstly, DNA sequences encoding the peptides are constructed according to conventional methods. The DNA sequences are constructed by PCR amplification using appropriate primers. Alternatively, the DNA sequences may be synthesized using any standard method known in the art, e.g., by use of an automated DNA synthesizer (such as are commercially available from Biosearch, Applied Biosystems, etc.). As examples, phosphorothioate oligonucleotides may be synthesized by the method of Stein et al. [Stein et al., 1988, Nucl. Acids Res. 16:3209 (1988)]. Methylphosphonate oligonucleotides can be prepared by use of controlled pore glass polymer supports [Sarin et al., 1988, Proc. Natl Acad. Sci. U.S.A. 85, 7448-7451 (1988)].

The constructed DNA sequences are inserted into vectors comprising one or more expression control sequences regulating expression of the DNA sequences to form recombinant expression vectors. Host cells are transformed or transfected with the vectors and the transformants or transfectants are cultured in a proper medium under proper conditions so that the DNA sequences express. By this way, substantially pure peptides encoded by the DAN sequences may be obtained from the cultures.

In another embodiment, the present invention is concerned a nucleic acid sequence coding a peptide conferring resistance to environmental stress, comprising a peptide fragment containing a sequence composed of 10 or more consecutive amino acid residues including five or more acidic amino acid residues, wherein the peptide fragment is derived from the C-terminal acidic tail of α-synuclein, or its derivative.

In more detail, the present invention relate to a nucleic acid sequence encoding a peptide conferring resistance to environmental stress, comprising (i) a peptide fragment containing a sequence composed of 10 or more consecutive amino acid residues including five or more acidic amino acid residues, wherein the peptide fragment is derived from SEQ ID NO:1 corresponding to amino acid residues 96-140 of the C-terminal acidic tail of α-synuclein, or its derivative, (ii) a peptide fragment containing a sequence composed of 10 or more consecutive amino acid residues including five or more acidic amino acid residues, wherein the peptide fragment is derived from SEQ ID NO:2 corresponding to amino acid residues 85-134 of the C-terminal acidic tail of β-synuclein, or its derivative, (iii) a peptide fragment containing a sequence composed of 10 or more consecutive amino acid residues including five or more acidic amino acid residues, wherein the peptide fragment is derived from SEQ ID NO:3 corresponding to amino acid residues 96-127 of the C-terminal acidic tail of γ-synuclein, or its derivative, or (iv) a peptide fragment containing a sequence composed of 10 or more consecutive amino acid residues including five or more acidic amino acid residues, wherein the peptide fragment is derived from SEQ ID NO:4 corresponding to amino acid residues 96-127 of the C-terminal acidic tail of synoretin, or its derivative.

A nucleic acid sequence encoding the peptide can be prepared by naturally isolating from cell, synthesizing artificially, or genetic recombination method.

In another embodiment, the present invention is concerned a nucleic acid sequence encoding a fusion protein comprising the peptide and a fusion partner protein

Such a nucleic acid sequence encoding a fusion protein may be prepared by genetic recombination methods, which are known in the art, ligating the nucleic acid sequence coding the peptide with nucleic acid sequence encoding the fusion partner protein.

In another embodiment, the present invention is concerned a recombinant vector comprising the nucleic acid sequence coding the peptide and a fusion partner protein.

The term “recombinant vector”, as used herein, means a vector capable of expressing a target protein in a suitable host cell, refers to a genetic construct that comprises essential regulatory elements to which a gene insert is operably linked thereto in such a manner as to be expressed in a host cell.

The term “operably linked”, as used herein, refers to a functional linkage between a nucleic acid expression control sequence (such as a promoter) and a second nucleic acid sequence coding for a target protein or RNA in a manner that allows general functions. For example, when a nucleic acid sequence coding for a protein or RNA is operably linked to a promoter, the promoter may affect the expression of a coding sequence. The operable linkage to a recombinant vector may be prepared using a genetic recombinant technique well known in the art, and site-specific DNA cleavage and ligation may be carried out using enzymes generally known in the art.

The vector useful in the present invention includes plasmid vectors, cosmid vectors and viral vectors. A suitable expression vector includes expression regulatory elements, such as a promoter, an operator, an initiation codon, a stop codon, a polyadenylation signal and an enhancer, and a signal sequence or leader sequence, and may be prepared in various constructs according to the intended use. The promoter of the vector may be constitutive or inducible. Also, the expression vector includes a selectable marker for selecting a host cell containing a vector, and, in the case of being replicable, includes a replication origin.

In another embodiment, the present invention is concerned transformants transfected with the recombinant vectors.

A transfection method of the vector includes any method of introducing a nucleic acid into the cell, and carried out using an appropriate technique well known in this art, may be performed by selecting suitable standard techniques according to host cells. These methods include, but are not limited to, electroporation, protoplast fusion, calcium phosphate (CaPO₄) precipitation, calcium chloride (CaCl₂) precipitation, agitation with silicon carbide fiber, and PEG-, dextran sulfate-and lipofectamine-mediated transformation.

Since expression levels and modification of proteins differ according to host cells, the most suitable host cell may be selected according to the intended use. Available host cells include, but are not limited to, prokaryotic cells such as Escherichia coli, Bacillus subtilis, Streptomyces, Pseudomonas, Proteus mirabillis or Staphylococcus. In addition, useful as host cells are lower eukaryotic cells, such as fungi (e.g., Aspergillus) and yeasts (e.g., Pichia pastoris, Saccharomyces cerevisiae, Schizosaccharomyces, Neurospora crassa), insect cells, plant cells, and cells derived from higher eukaryotes including mammals.

In another embodiment, the present invention is concerned a method of producing the fusion proteins, comprising transforming a host cell with a recombinant vector including a nucleotide sequence encoding the fusion protein; culturing the resulting transformant; and isolating and purifying the fusion protein expressed from the transformant.

The Culture of transformants transformed with the recombinant vectors are carried out under adjusted condition, which fusion protein is able to be expressed. Culture conditions may be easily adjusted by those skilled in the art. Typically, a medium used in the culturing should contain all nutrients essential for the growth and survival of cells. The medium should contain a variety of carbon sources, nitrogen sources and trace elements.

For example, cells transformed with the recombinant vector are harvested and sonicated using ultrasonicator, and then supernant are obtained through ultracentrifugation removing the cell debris. In case of protein secreted, can be obtained from harvested culture media. In case of protein expressed forming inclusion body, can be obtained by an additional process including dissolution, denaturation in a suitable solution and refolding using a refolding agent (Kohno, Meth. Enzym., 185:187-195, 1990). Redox system such as glutathione, dithiothreitol, β-mercaptoethanol, cystein, cystamin and refolding agents such as urea, guanidine, arginine acan be used. Refolding agent may be used with some salts.

The protein produced by the transformants, may be isolated and purified by salting out (e.g., ammonium sulfate precipitation, sodium phosphate precipitation, etc.), solvent precipitation (e.g., protein fraction precipitation using acetone, ethanol, etc.), dialysis, various chromatographies, such as gel filtration, ion exchange and reverse phase column chromatography, and ultrafiltration. These techniques are used singly or in combinations of two or more to obtain a fusion protein

Now, the present invention will be described in detail by the following examples. However, the examples are for illustration of the present invention and do not limit the scope of the present invention thereto.

### EXAMPLES

### Example 1

### Preparation of GST-Synuclein Fusion Constructs and Expression Vectors

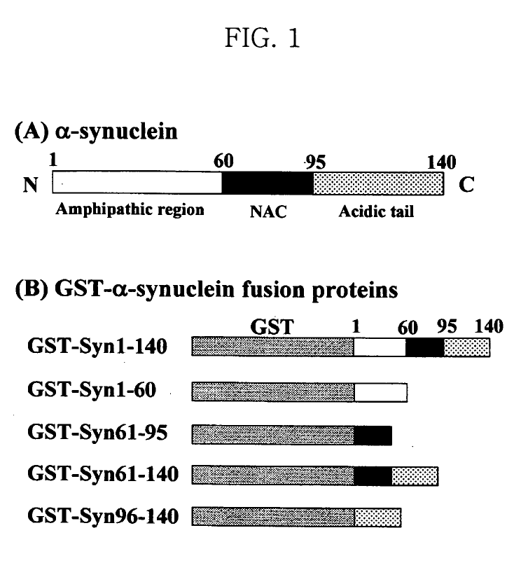

α-synuclein consists of three distinct regions, the N-terminal amphipathic region (residues 1-60; FIG. 1a), the hydrophobic NAC region (residues 61-95; FIG. 1a), and the C-terminal acidic region (residues 96-140; FIG. 1a). Five GST-synuclein fusion constructs encoding GST-Syn1-140(SEQ ID NO:76), a fusion protein of the entire region of α-synuclein and GST, GST-Syn1-60(SEQ IDNO:77), a fusion protein of the amphipathic region and GST, GST-Syn61-95(SEQ ID NO:78), a fusion protein of the NAC region and GST, GST-Syn61-140(SEQ ID NO:79), a fusion protein of the NAC plus acidic tail region and GST, and GST-Syn96-140(SEQ ID NO:80), a fusion protein of the acidic tail region and GST, were synthesized, respectively (FIG. 1b).

GST-α-synuclein fusion constrcts were prepared by PCR amplification of the α-synuclein gene with the specific primers described below and ligating the amplified DNAs after GST gene in the pGEX expression vector (Amersham Pharmacia Biotech). The protein coding regions of the full-length α-synuclein (residues 1-140) was amplified by PCR with the primer 1 (SEQ ID NO:19) conaining the underlined Bg1II restriction site and the primer 2 (SEQ ID NO:20) containing the underlined Sa1I restriction site and the amino-terminal amphipathic part (residues 1-60) was amplified by PCR with the primer 1 (SEQ ID NO: 19) and the primer 3 (SEQ ID NO:21) containing the underlined Sa1I restriction site. The protein coding regions of the NAC (residues 61-95) was amplified by PCR with the primer 4 (SEQ ID NO:22) containing the underlined Bg1II restriction site and the primer 5 (SEQ ID NO:23) containing the underlined Sa1II restriction site and the NAC plus acidic tail (residues 61-140) was amplified by PCR with the primer 4 (SEQ ID NO:22) and the primer 2 SEQ ID NO:20). The protein coding region of the C-terminal acidic tail (residues 96-140) was amplified by PCR with the primer 6 (SEQ ID NO:24) containing the underlined KpnI restriction site and the primer 7 (SEQ ID NO:25) containing the underlined Sa1I restriction site. Sequences of the used primers are shown in Table 1.

TABLE 1

| | | | SEQ |
|---|---|---|---|
| NO. | Primer | DNA Sequence | ID NO |
| 1 | Sense | 5′-CGCTCGAGCCAGATCTGCCATGGATGTA | 19 |
| | | TTCATGA-3′ | |
| 2 | Antisense | 5′-GCGCAAGCTTGTCGACTTAGGCTTCAGG | 20 |
| | | TTCGTAGT-3′ | |
| 3 | Antisense | 5′-GCGCAAGCTTGTCGACCTATTTGGTCTT | 21 |
| | | CTCAGCCAC-3′ | |
| 4 | Sense | 5′-GCGCAGATCTCATATGGAGCAAGTGAC | 22 |
| | | A-3′ | |
| 5 | Antisense | 5′-GCGCAAGCTTGTCGACCTAGACTTAGCC | 23 |
| | | AGTGGC-3′ | |
| 6 | Sense | 5′-GCGCGGTACCGAGATCTGGATGAAAAAG | 24 |
| | | GACCAGTTGGGC-3′ | |
| 7 | Antisense | 5′-GCGCAAGCTTGTCGACTTAGGCTTCAGG | 25 |
| | | TTCGTAGT-3′ | |

The amplified DNAs were purified by electrophoresis using 1% agarose gel, digested with restriction enzymes, then ligated into the restriction enzyme sites of the pGEX vector (Pharmacia Biotech, Buckingamshire, UK) to construct the expression vectors. All constructs were verified for their sequences by DNA sequencing.

### Example 2

### Bacterial Expression and Purification of GST-Synuclein Fusion Proteins

The expression vectors constructed in Example I for expression of GST-synuclein fusion proteins were transformed into the E. coli strain, BL21 (DE3) plysS (Invitrogen). The transformed bacteria were grown in a LB medium containing 0.1 mg/ml ampicillin at 37° C. to an A₆₀₀ of 0.8, induced with 0.5 mM IPTG and then, cultured for a further 4 hours. The culture was then centrifuged at 10,000 rpm for 10 minutes to harvest cells. The cells were resuspended in phosphate-buffered saline (PBS, pH 7.4) and disrupted by ultrasonication. After removing the cell debris, the supernatants were purified by affinity chromatography. That is, the supernatants were passed through a glutathione-Sepharose 4B column (Peptron, Taejeon, Korea) equilibrated with PBS. After washing with PBS, the fusion proteins were eluted with 10 mM GSH (Sigma, St. Louis, Mo.). The eluted GST-synuclein fusion proteins were further purified on an FPLC gel-filtration column and concentrated by the Centricon condencer (Amicon, Beverly, Mass.).

### Example 3

### Thermal Behavior of α-Synuclein and GST Protein

α-synuclein is an “intrinsically unstructured protein” which almost lacks a regular secondary structure and contains a very high portion of random-coil (Plaxco K. W. and Gro, M., Nature, 386, 657-658 (1997); Wright P. E. and Dyson H., J., J. Mol. Biol., 293, 321-331 (1999); Kim J., Molecules and Cells, 7, 78-83 (1997); and Weinreb P. H. et al., Biochemistry, 35, 13709-13715 (1996)). Previous studies have shown that intrinsically unstructured proteins, such as α-synuclein and αₛ-casein, are heat-resistant since the proteins have a similar unfolded conformation regardless of the temperature and their unfolded conformation is stable at high temperatures as well as at room temperature (Kim T. D. et al., Biochemistry, 39, 14839-14846 (2000)). Therefore, the thermal behavior of α-synuclein and GST protein was initially compared using a qualitative heat-induced protein aggregation assay. The GST and α-synuclein proteins used in this example were prepared by transforming pGEX vector and pRK172 expression vector containing GST and α-synuclein genes, respectively, into E coli (Jakes et al., FEBS Letters 345, 27-32 (1994)). The recombinant GST protein was purified by the same method as described in Example 2 and the recombinant α-synuclein was purified according to the known method (Kim J., Molecules and Cells, 7, 78-83 (1997); Paik S. R. et al., Arch. Biochem. Biophys., 344, 325-334 (1997)).

The heat-induced aggregation of GST and α-synuclein protein was qualitatively assayed by SDS polyacrylamide gel after heat treatment of the samples. Each protein suspended in PBS (0.6 mg/ml) was heated in a boiling water bath for 10 minutes and cooled in the air. The protein samples were centrifuged at 15,000 rpm for 10 minutes and the supernatants were analyzed on a 12% SDS polyacrylamide gel. The protein bands were stained with Coomassie Brillinant blue R250.

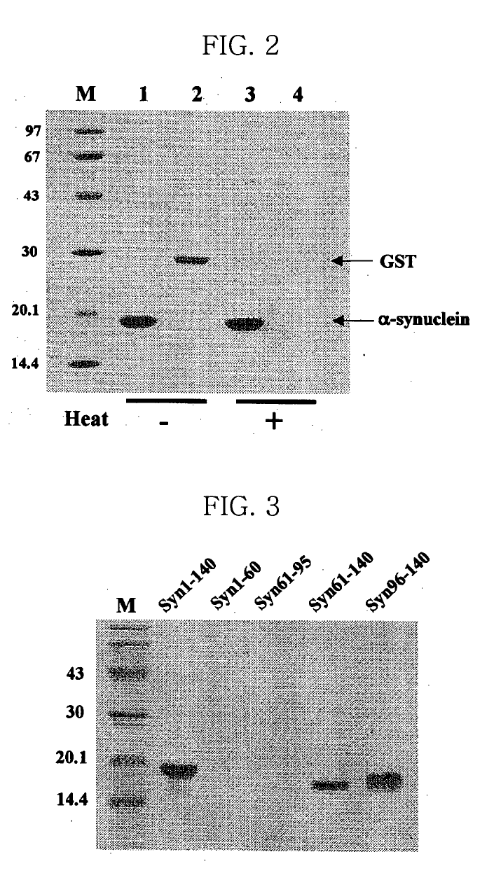

As expected, α-synuclein did not precipitate upon heat treatment, whereas the GST protein did (FIG. 2). For α-synuclein, the protein bands were observed when both heat-treated and non-heat-treated. However, for GST protein, the protein bands were observed when non-heat-treated but were not observed after heat-treated. Thus, it was noted that α-synuclein is a heat-resistant protein while GST is a heat-labile protein. Such experimental results were reproducible regardless of the pH and salt concentration of the buffer solution and the protein concentration (data not shown).

### Example 4

### Thermal Behavior of α-Synuclein Deletion Mutants

Next, a series of deletion mutants were used to determine the domain, inducing heat resistance of α-synuclein. The GST-synuclein fusion proteins prepared in Example 2 were treated with 1 unit of thrombin per 1 mg of protein for 2 hours at room temperature to cleave the α-synuclein fragments from the GST fusion proteins. The resulting α-synuclein deletion mutants were examined for their thermal stability.

According to the same method with Example 3, the cleaved products obtained by thrombin digestion were examined for their thermal stability. The obtained α-synuclein deletion mutants include two deletion mutants (Syn61-140, Syn96-140), each containing the ATSα (residues 96-140), a deletion mutant containing α-synuclein N-terminal (Syn-160) and a deletion mutant containing the hydrophobic NAC region (Syn61-95).

Wild type (Syn1-140) and two deletion mutants containing the ATSα (Syn61-140, Syn96-140) did not precipitate and hence, protein bands were observed in an analysis using an SDS polyacrylamide gel after heat treatment. This indicated that the two proteins are heat-resistant. In contrast, the N-terminal part of α-synuclein (Syn1-60) and the NAC peptide (Syn61-95) appeared to precipitate upon heat treatment and hence, no protein band was observed (FIG. 3). From these results, only the deletion mutants containing the ATSα were found to be heat-resistant. Accordingly, it was noted that the ATSα is responsible for the heat resistance. Consistent with data of the inventors, previous studies have shown that C-terminally truncated α-synuclein proteins and the NAC peptide assembled into filaments much more readily than the wild type protein (Serpell L. C. et al., Proc. Natl. Acad. Sci. USA, 97, 4897-4902 (2000); Crowther R. A. et al., FEBS Letters, 436, 309-312 (1998); Han H. et al., Chem. Biol., 2, 163-169 (1995); and Iwai A. et al., Biochemistry, 34, 10139-10145 (1995)). Overall, it appears likely that C-terminally truncated α-synuclein mutant proteins are less stable at room temperature and higher temperature than both the wild type and mutant proteins containing the C-terminal acidic tail. Thus, it is noted that the ATSα plays a very important role for thermosolubility of α-synuclein.

### Example 5

### Thermal Behavior of GST-Synuclein Fusion Proteins

The thermal behaviors of GST-synuclein fusion proteins, prepared as in Example 2, were investigated. Using the same method as described in Example 3, the GST-α-synuclein fusion proteins were boiled in a boiling water bath for 10 minutes. The protein solutions were centrifuged and the supernatants were analyzed on a SDS polyacrylamide gel. Also, the thermal behaviors of GST-α-synuclein fusion proteins were quantitatively by monitoring absorbance at 360 nm according to time (Lee G. J. and Vierling E., Method Enzymol., 290, 360-65 (1998); Horwitz J. Proc. Natl. Acad. Sci. USA 89, 10449-53 (1992)).

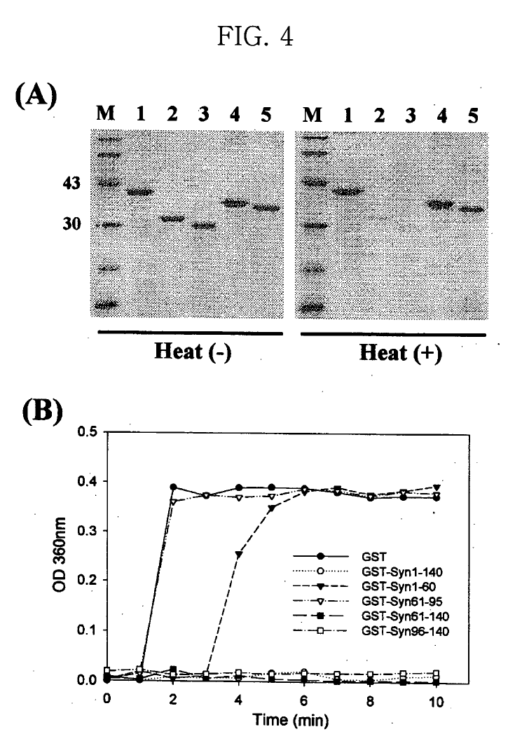

In the experiment, as shown in FIG. 4a, GST-Syn1-140, GST-Syn61-140 and GST-Syn96-140 shows protein bands both before and after heat treatment, indicating that these proteins did not precipitate upon heat treatment. Therefore, it is noted that they are heat-resistant. Whereas, for GST-Syn1-60 and GST-Syn61-95, protein bands were observed before heat treatment, but not observed after heat treatment. Therefore, it is noted that these proteins are heat-labile and had completely precipitated upon heat treatment.

Also, the heat-induced aggregation of the GST-synuclein fusion proteins was quantitatively analyzed by measuring the turbidity at 65° C. according to time. As shown in FIG. 4b, the OD₃₆₀ of the GST protein drastically increased 2 minutes after heat treatment, and most of the protein had aggregated by 3 minutes. GST-Syn61-95 behaved similarly to the GST protein, and resulted in complete aggregation. GST-Syn1-60 also resulted in complete aggregation after heat treatment, although aggregation of this protein was relatively delayed. Consistent with the results in FIG. 4a, there was no evidence of any protein aggregation for GST-Syn1-140, GST-Syn61-140 and GST-Syn96-140 even after heat treatment of 30 minutes. Interestingly, these three heat-resistant GST-synuclein fusion proteins all contain the ATSα. From these results, it is noted that a heat-labile protein can be transformed into a heat-resistant protein by introducing the ATSα.

### Example 6

### PI and Hydropathy Values of α-Synuclein Deletion Mutants, GST and GST-Synuclein Fusion Proteins

Previously, many of the heat-resistant proteins from Jurkat T cell lysates and human serum were reported to be highly acidic proteins. This implies that the pI value may be related to heat-resistance of proteins (Kim T. D., et al., Molecules and Cells, 7, 78-83 (2000)). The solubility of proteins may play an important role in determining the heat-resistance, since highly charged proteins would be soluble even at higher temperatures. To confirm this hypothesis, the pI and hydropathy values of α-synuclein deletion mutants were compared with those of GST and GST-synuclein fusion proteins (Table 2). The pI and hydropathy values were calculated using ProtParam program.

From the results, as shown in Table 2, heat-resistant proteins, such as α-synuclein, Syn61-140, Syn96-140, GST-Syn1-140, GST-Syn61-140 and GST-Syn96-140, have abnormally low pI and hydropathy values. On the other hand, the heat-labile proteins with the exception of Syn61-95 show much higher values. Interestingly, Syn61-95, a heat-labile peptide shows a very low pI value but it has an extremely high hydropathy value. Therefore, it is possible that highly charged proteins with a low hydropathy value possesses an advantage in resisting heat-induced protein aggregation.

TABLE 2

| Protein | Temp. Rxn | Pi Value^{a} | Hydropathy^{p} |
|---|---|---|---|
| α-Synuclein | HR^{c} | 4.67 | −0.403 |
| Syn1-60 | HL^{d} | 9.52 | −0.188 |
| Syn61-95 | HL | 4.53 | 0.726 |
| Syn61-140 | HR | 3.85 | −0.564 |
| Syn96-140 | HR | 3.76 | −1.567 |
| GST | HL | 6.18 | −0.390 |
| GST-Syn1-140 | HR | 5.25 | −0.378 |
| GST-Syn1-60 | HL | 7.64 | −0.349 |
| GST-Syn61-95 | HL | 6.01 | −0.244 |
| GST-Syn61-140 | HR | 4.95 | −0.435 |
| GST-Syn96-140 | HR | 4.85 | −0.560 |

^{a}pI value was calculated by using ProtParam program (www.expasy.ch).
^{b}Hydropathy value was calculated by using ProtParam program (www.expasy.ch).
^{c}HR, heat-resistant
^{d}HL, heat-labile

### Example 7

### Effect of Divalent Cation Binding on GST-Synuclein Fusion Proteins

Several divalent cations, such as Cu²⁺ and Ca²⁺, are known to bind specifically to the ATSα region with a dissociation constant of the micromolar ranges (Paik S. R. et al., Biochem. J., 340, 821-8 (1999); and Nielsen M. S. et al., J. Biol. Chem., 276, 22680-22684). Zn²⁺ also is known to bind specifically to α-synuclein, although the binding sites are not yet identified (Paik S. R. et al., Biochem. J., 340, 821-8 (1999); and Kim T. D. et al., Biochemistry, 39, 14839-14846 (2000)). Since the ATSα is important for heat-resistance of proteins, the effect of the divalent cation binding on the heat-induced aggregation of GST-synuclein fusion proteins containing the ATSα was investigated. As divalent cations, CaCl₂, MgCl₂ and ZnCl₂ were used. The GST-Syn1-140, GST-Syn61-140 and GST-Syn96-140 fusion proteins were diluted to a final concentration of 0.2 mg/ml in 20 mM Tris-HCl buffers containing 0 to 1.0 mM of respective divalent cations. The protein solutions were reacted at 65° C. for 30 minutes and their apparent absorbances were measured at 360 nm.

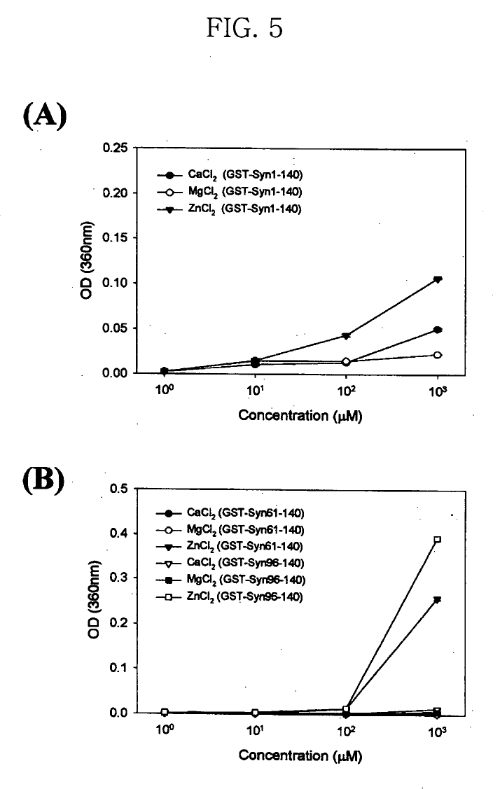

From the results as shown in FIG. 5a and FIG. 5b, it was found that low concentrations of the divalent cations do not affect the heat-induced aggregation of the fusion proteins. However, high concentrations significantly increased the protein aggregation, although the fusion proteins did not completely precipitated. Particularly, Zn²⁺ appeared to be most effective for enhancing the heat-induced protein aggregation. The dissociation constants between α-synuclein and the divalent cations were considerably low, and most proteins were affected by a high concentration of metal ions. Therefore, the results suggest that the specific binding of the divalent cations at the ATSα region does not affect the thermal behavior of the fusion proteins. However, it was noted that nonspecific binding of the metal ions at a high concentration induces more protein aggregation during heat treatment.

### Example 8

### GST Activity of Synuclein Fusion Proteins after Heat Treatment

Unlike the wild type GST protein described in the foregoing Examples, GST-synuclein fusion proteins containing the ATSα were found to be heat resistant. This suggests that the heat-labile protein could be transformed into a heat-resistant protein simply by introducing the ATSα. Subsequently, whether or not the heat-resistant GST-fusion proteins could keep the enzymatic activity after heat treatment was investigated. The GST and GST-synuclein fusion proteins were boiled in a water bath for 10 minutes and cooled in the air at room temperature. The enzymatic activities of these heat-treated proteins were then compared. The enzymatic activity was assayed using a chromogenic substrate, 1-chloro-2,4-dinitro benzen (DTNB) (Habig W. H. et al., J. Biol. Chem., 249, 7130-7139 (1974)). The purified GST and GST-synuclein fusion proteins were diluted into the substrate solution (1 mM GSH and 2 mM DTNB dissolbed in 0.1 M phosphate buffer, pH 7.4) to a final concentration of 20 pg/ml and incubated at 37° C. for 10 minutes. Upon completion of incubation, the enzymatic activity was assayed by measuring absorbance at 350 nm. The absorbance was measured on a Spectramax 250 microplate reader (Molecular Devices, Calif., USA).

From the results, as shown in FIG. 6a, all the GST and GST-fusion proteins completely lost their enzymatic activity under these stringent conditions. Subsequently, the thermostabilities of GST and GST-Syn96-140 were quantitatively measured by thermal inactivation curves (FIG. 6b), which were used to determine the T₅₀ values, the temperatures at which 50% of initial enzymatic activity was lost after heat treatment. As shown in FIG. 6b, the T₅₀ of GST-Syn96-140 is about 2° C. higher than that of GST. Interestingly, the thermal inactivation of GST is well correlated with the thermal aggregation of the protein. It is noted that the introduced ATSα is able to protect the enzyme from the thermal inactivation by preventing the thermal aggregation of the fusion protein.

### Example 9

### Heat-Induced Structural Changes of GST-ATSα

Previously, heat-induced secondatry structural changes of α-synuclein assayed by CD analysis has been reported (Kim T. D. et al., Biochemistry, 39, 14839-14846 (2000)). The CD spectrum of α-synuclein indicated that the protein almost completely lacks secondary structural elements. Also, it was shown that the CD spectrum of α-synuclein at 100° C. was slightly different from that at 25° C. but it represented the characteristics of random-coiled polypeptides. Consistent with these results, a linear temperature-dependence of the CD signal, often seen with unfolded peptides, was observed.

The present inventors analyzed the secondary structural changes of GST due to thermal denaturation by measuring CD spectra of GST and the GST-Synclein fusion protein. The CD spectra were recorded on a Jasco-J715 spectropolarimeter (Jasco, Japan) equipped with a temperature control system in a continuous mode. The far-UV CD measurements were carried out over the wavelength range of 190 to 250 nm with 0.5 nm bandwidth, a one second response time and a 10 nm/minute scan speed at 25° C. and 100° C. The spectra shown are an average of five scans that were corrected by subtraction of the buffer signal. The CD data were expressed in terms of the mean residue ellipticity, [θ] (deg.cm².dmol⁻¹). The protein samples for CD measurements were prepared in 10 mM sodium phosphate buffer, unless otherwise specified, and all spectra were measured in a cuvette with a path length of 0.1 cm.

Thermal denaturation experiments were performed using a heating rate of IOC/min and a response time of 1 second. The thermal scan data were collected from 25 to 100° C. The concentrations of GST and the GST-Syn96-140 were 0.1 mg/ml and 0.3 mg/ml, respectively. The CD spectra were measured every 0.5° C. at a wavelength of 222 nm, unless otherwise specified. The reversibility of the thermal transition was examined by comparing a new scan recorded by decreasing the temperature and another scan recorded by cooling the thermally unfolded protein sample.

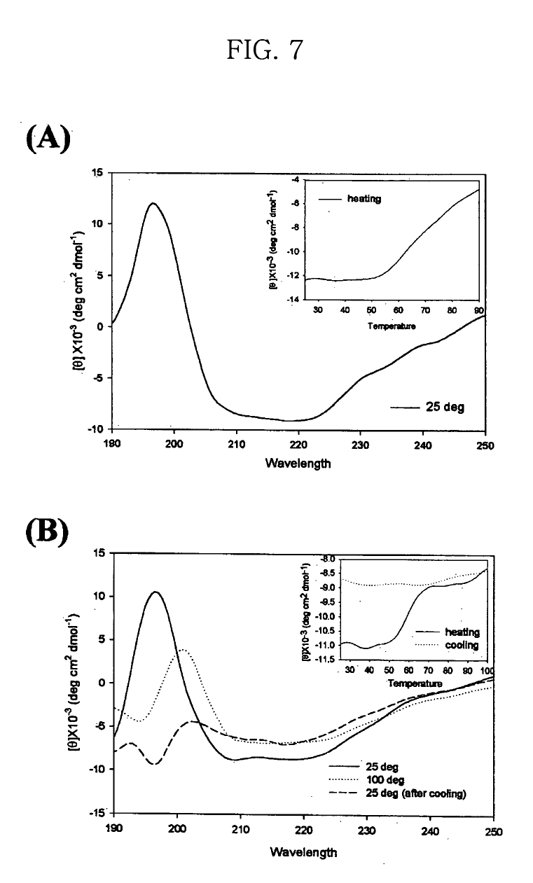

From the CD spectrum of GST at 25° C., as shown in FIG. 7a, it was found that the protein contains well ordered secondary structural elements. However, at 100° C., the far-UV CD spectrum almost disappeared due to protein precipitation (data not shown). Through the heat-induced changes in the ellipticity of the GST at 222 nm, the Tm of GST was found to be approximately 70° C. The GST had completely precipitated at 100° C. and a CD signal was not observed at 222 nm, which indicates that GST had irreversibly precipitated (data not shown). These results confirm that the GST protein is a typical heat-labile protein which unfolds and precipitates as the temperature is increased.

The far-UV CD spectra of GST-Syn96-140 are shown in FIG. 7b. The far-UV CD spectrum of GST-Syn96-140 at room temperature (solid line) indicates that the protein contains well-ordered secondary structural elements. The CD spectrum showed a decrease in these elements at 100° C. but the overall shape was unchanged (dotted line). These results mean that heating does not lead to complete unfolding. Interestingly, a new absorption band at 195 nm appears, which is characteristic of random-coiled polypeptides. After cooling the protein solutions, the far-UV CD spectrum is distinguishable from the initial one (dashed line), which indicates that the conformation of GST-Syn96-140 may be irreversibly changed. The CD spectrum of the heat-treated GST-Syn96-140 at room temperature rather resembles that obtained at 100° C., which indicates that the protein consists of two distinct domains: one with regular secondary structural elements and the other with a random-coil like conformation. To confirm the conformational changes induced by heating, the GST-Syn96-140 melting curves were measured according to temperature. The heat-induced changes in the ellipticity at 222 nm are presented in FIG. 7b. Interestingly, the heat-induced unfolding of GST-Syn96-140 appeared to take place in two stages. The first transition was observed at 62° C. and the second transition observed at 95° C. As expected, the temperature curves of GST-Syn96-140 appeared to be irreversible (dotted line).

GST is a heat-labile protein, while GST-Syn96-140 is a heat-resistant protein. To compare the stability of the two proteins, it would be useful to determine the Tm of both proteins. However, it is difficult to directly compare the Tm values of GST-Syn96-140 and GST, since these proteins contain different number of peptide domains. Interestingly, the Tm value of GST-Syn96-140 (62° C. for the first transition) appears to be slightly lower than that of GST (70° C. for the first transition). Since the Tm of a given protein is related to the change in the free energy between the native and thermally denatured state of the protein, the Tm has been used as a thermodynamic parameter of the conformational stability of the protein. Therefore, it is noted that introduction of the ATSα to the C-terminus of GST is favorable for protein stability against environmental stress such as increased temperature and consequently for heat-resistancy, but unfavorable for intrinsic thermal stability of the protein.

### Example 10

### Effect of the ATSα on pH- and Metal-Induced Protein Aggregation

The pH-induced aggregation of GST and GST-Syn96-140 was investigated by measuring the turbidity at 65° C. according to time. The measurement of the turbidity was carried out by monitoring the apparent absorbance at 360 nm according to time. Each protein was diluted to a final concentration 0.2 mg/ml in buffers with different pH values. The buffers used were 0.1 M acetate (pH 4.0 and 5.0), 0.1 M citrate (pH 6.0), and 0.1 M Tris-HCl (pH 7.4). The protein solutions diluted in buffers were incubated for 1 hour at room temperature and their apparent absorbance were measured in a Beckman spectrophotometer (DU650, Beckman). The metal-induced aggregation of GST and GST-Syn96-140 was similarly assessed. Each protein was diluted to a final concentration of 0.2 mg/ml in 20 mM Tris-HCl buffers containing 0 to 1.0 mM of Zn²⁺, or Cu²⁺. The protein solutions were incubated for 30 minutes at room temperature and their apparent absorbances at 360 nm were measured.

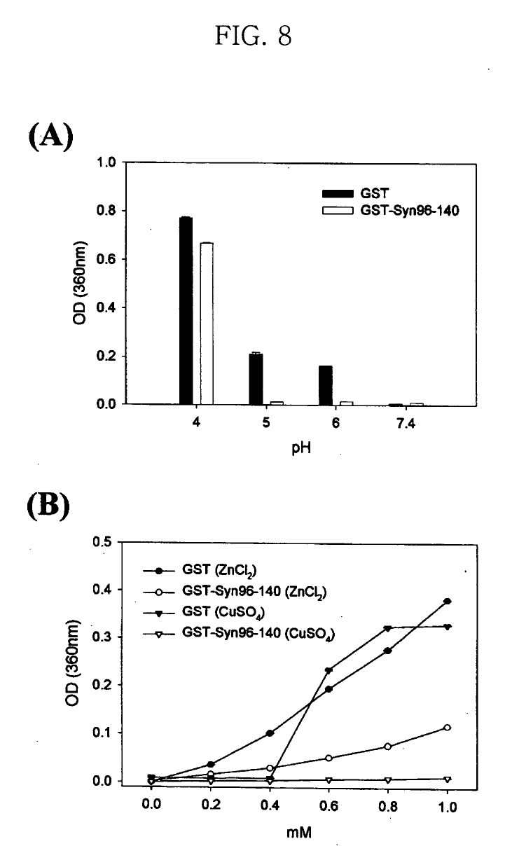

The results of the pH-induced aggregation of the proteins were shown in FIG. 8a. The OD₃₆₀ of the GST protein steadily increased from pH 7.4 to pH 5.0 and reached maximum value at pH 4.0. On the other hand, the OD₃₆₀ of GST-Syn96-140 was not changed until pH 5.0, but drastically increased at pH 4.0, perhaps due to the neutralization of the acidic tail. From these results, it is noted that the ATSα does not show sufficient protection effect under very acidic conditions but can completely protect GST from aggregation induced by pH 4.5 or higher. The results of the metal-induced aggregation of the proteins were shown in FIG. 8b. The ATSα also appeared to protect GST from metal-induced aggregation. The OD₃₆₀ of the GST protein steadily increased when it was treated with 0.2 to 1.0 mM Zn²⁺, while the OD₃₆₀ of GST-Syn96-140 was always much lower than that of GST. In particular, Cu²⁺-induced protein aggregation was completely blocked by introducing ATSα. From these results, it is noted that the ATSα can also protect GST from metal-induced aggregation.

### Example 11

### Effect of the ATSα on Stress-Induced Aggregation of DHFR

In order to examine whether any fusion proteins with the ATSα (Syn96-140) other than GST-ATSα show resistance to environmental stresses, the present inventors constructed a DHFR-synuclein fusion protein, DHFR-ATSα (SEQ ID NO:81), which contains the ATSα at the C-terminus.

The protein coding region of DHFR was subcloned into an E. coli expression vector, pRSETA, using BamHI and HindII restriction sites (pDHFR). The protein coding region of the ATSα (residues 96-140) was amplified by PCR with the 5′-oligonucleotide primer (Table 3, SEQ ID NO:26) containing the underlined KpnI restriction site and 3-oligonucleotide primer (SEQ ID NO:27) containing the underlined SalI restriction site. The amplified DNAs were gel purified, digested with appropriate enzymes, ligated into the pDHFR vector which had been digested with appropriate restriction enzymes, and gel purified. The resulting expression vector (pDHFR-ATSα) was verified by DNA sequencing.

TABLE 3

| | | |
|---|---|---|
| NO. | Primer | Sequence |

| | | | |
|---|---|---|---|
| 8 | Sense | GCGCGGTACCAAGGACCAGTTG | (SEQ ID NO:26) |
| | | GGCAAGAATG | |
| 9 | Antisense | GCGCGTCGACTTAGGCTTCAGG | (SEQ ID NO:27) |
| | | TTCGTAGT | |

The expression vector (pDHFR-ASTα) was transformed into the E. coli strain, BL21 (DE3), for protein expression. The transformed bacteria were grown in a LB medium containing 0.1 mg/ml ampicillin at 37° C. to an A₆₀₀ of 0.8. 0.5 mM IPTG was added to the medium, which was cultured for a further 4 hours. The culture was centrifuged at 10,000 rpm for 10 minutes to harvest cells. The cells were resuspended in phosphate-buffered saline (PBS, pH 7.4), and disrupted by ultrasonication. After removing the lysed strains, the supernatants were loaded onto a Ni-NTA column equilibrated with a loading buffer (50 mM phosphate buffer (pH 8.0) containing 0.3M NaCl and 10 mM imidazole). After washing with the loading buffer, the protein was eluted with 250 mM imidazole in the same buffer. The DHFR-ATSα was further purified on an FPLC gel-filtration column. The purified protein was concentrated and buffer-changed by Centricon (Amicon, Beverly, Mass.).

The heat resistance of the DHFR-ATSα fusion protein was compared with that of DHFR. Each protein suspended in PBS (0.2 mg/ml) was heated in boiling water baths at 65° C. and 100° C. for 10 minutes each and cooled in the air. The protein samples were centrifuged at 15,000 rpm for 10 minutes and the supernatants were analyzed on a 12% SDS polyacrylamide gel. The protein bands on the SDS polyacrylamide gel were stained with Coomassie Brillinant blue R250 to be visible.

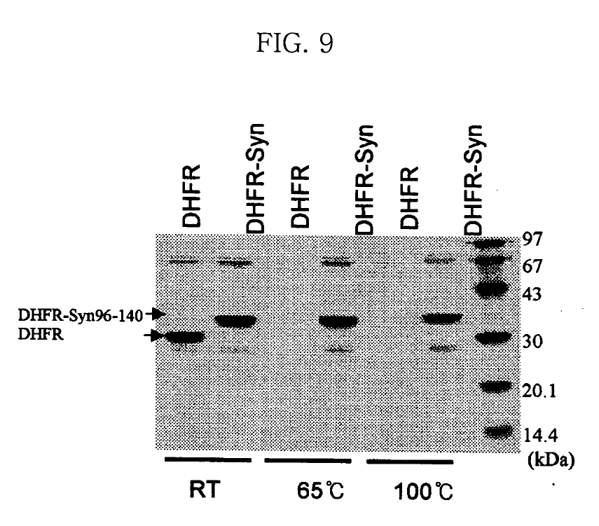

As shown in FIG. 9, for DHFR-ATSα, the protein bands were observed both before heat treatment and after heat treatment at 65° C. and 100° C., which indicates that no precipitation due to heat treatment takes place. On the other hand, DHFR, the protein bands were observed before heat treatment but not after heat treatment. This indicates that the protein completely precipitated by heat treatment and is heat-labile. Thus, it was noted that wild type DHFR is a heat-labile protein which readily precipitates by thermal stress while DHFR-ATSα according to the present invention has a high heat-resistance. That is, it is demonstrated that ATSα is a peptide capable of providing heat resistance to DHFR and other proteins, as well as GST.

### Example 12

### Heat-Resistance of GST-Synuclein Fusion Proteins with Peptide Fragments Derived from the ATSα

The C-terminal acidic tail of α-synuclein (ATSα) is composed of 45 amino acids (residues 96-140), and 15 Glu/Asp residues are scattered through the ATSαregion. The present inventors examined whether GST-synuclein fusion proteins with peptide fragments derived from the ATSα have heat-resistance. For this, a series of GST-synuclein fusion proteins with peptide fragments derived from the ATSα were constructed by ligating the gene coding fragment of ATSα into pGEX vector. DNAs encoding the fragment of the ATSα were synthesized with olignucleotides described in Table 4 (SEQ ID NOS:28-35) using an automatic DNA synthesizer.

TABLE 4

| | | |
|---|---|---|
| NO. | Primer | Sequence |

| | | | |
|---|---|---|---|
| 10 | Sense | GATCCAATGAAGAAGGAGCCCC | (SEQ ID NO:28) |
| | | ACAGGAAGGCATTCTGGAAGAT | |
| | | TAAG | |
| 11 | Antisense | AATTCTTAATCTTCCAGAATGC | (SEQ ID NO:29) |
| | | CTTCCTGTGGGGCTCCTTCTTC | |
| | | ATTG | |
| 12 | Sense | GATCCGAAGATATGCCTGTAGA | (SEQ ID NO:30) |
| | | TCCTGACAATGAGGCTTATGAA | |
| | | TAAG | |
| 13 | Antisense | AATTCTTATTCATAAGCCTCAT | (SEQ ID NO:31) |
| | | TGTCAGGATCTACAGGCATATC | |
| | | TTCG | |
| 14 | Sense | GATCCGATCCTGACAATGAGGC | (SEQ ID NO:32) |
| | | TTATGAAATGCCTTCTGAGGAA | |
| | | GGGTATCAAGACTACGAACCTG | |
| | | AAGCCTAAG | |
| 15 | Antisense | AATTCTTAGGCTTCAGGTTCGT | (SEQ ID NO:33) |
| | | AGTCTTGATACCCTTCCTCAGA | |
| | | AGGCATTTCATAAGCCTCATTG | |
| | | TCAGGATCG | |
| 16 | Sense | GATCCGAGGAAGGGTATCAAGA | (SEQ ID NO:34) |
| | | CTACGAACCTGAAGCCTAAG | |
| 17 | Antisense | AATTCTTAGGCTTCAGGTTCGT | (SEQ ID NO:35) |
| | | AGTCTTGATACCCTTCCTCG | |

GST-Syn103-115 was constructed using an oligonucleotide of SEQ ID NO:28 as sense and oligonucleotide of SEQ ID NO:29 as antisense. GST-Syn114-126 was constructed using oligonucleotides represented by SEQ ID NO:30 and SEQ ID NO:31. GST-Syn119-140 was constructed using oligonucleotides represented by SEQ ID NO:32 and SEQ ID NO:33. GST-Syn130-140 was constructed using oligonucleotides represented by SEQ ID NO:34 and SEQ ID NO:35. The synthesized sense and antisense DNA pairs were annealed and ligated into BamHI and EcoRI restriction sites of the pGEX vectors to construct a series of expression vectors of GST-ATSα deletion mutants (FIG. 10a), as follows: GST-Syn103-115 containing 13 amino acids of ATSA (residues 103-115)(SEQ ID NO:82); GST-Syn114-126 containing 13 amino acids of ATSA (residues 114-126) (SEQ ID NO:83); GST-Syn119-140 containing 22 amino acids of ATSα (residues 119-140) (SEQ ID NO:84); and GST-Syn130-140 containing 11 amino acids of ATSα (residues 130-140) (SEQ ID NO:85). All the expression vectors (pGST-Syn103-115, pGST-Syn114-126, pGST-Syn119-140 and pGST-Syn130-140) were verified for their sequences by DNA sequencing. The expression vectors pGST-Syn103-115, pGST-Syn114-126, pGST-Syn119-140 and pGST-Syn130-140 were transformed into the E. coli BL21 (DE3) and the resulting recombinant proteins were purified by affinity chromatography using glutathione-Sepharose 4B beads. The GST-synuclein fusion proteins with peptide fragments derived from ATSα were further purified on an FPLC gel-filtration column.

The GST-synuclein fusion proteins with peptide fragments derived from ATSα were examined for heat-resistance. Each protein suspended in PBS (0.2 mg/ml) was heated in boiling water baths for 10 minutes and cooled in the air. The protein samples were centrifuged at 15,000 rpm for 10 minutes and the supernatants were analyzed on a 12% SDS polyacrylamide gel. The protein bands on the SDS polyacrylamide gel were stained with Coomassie Brillinant blue R250 to be visible.

As shown in FIG. 10b, when GST-synuclein fusion proteins with peptide fragments derived from ATSα were thermally treated at a high concentration (0.6 mg/ml), GST-Syn96-140 containing the entire region of ATSα and GST-Syn119-140 containing 22 amino acids of ATSα did not precipitate at all, while GST-Syn103-115, GST-Syn114-126 and GST-Syn130-140 containing 11-13 amino acids partially precipitated. On the other hand, when these deletion mutants of the GST-ATSα fusion proteins were thermally treated at a low concentration (0.2 mg/ml), all the proteins did not aggregate at all (data not shown).

Also, the thermal behaviors of GST-synuclein fusion proteins with peptide fragments derived from ATSA were quantitatively analyzed by monitoring absorbance at 360 nm according to time while setting the concentration of each protein at 0.2 mg/ml at 65° C. (Lee G. J. and Vierling E., Method Enzymol., 290, 360-65 (1998); and Horwitz J. Proc. Natl. Acad. Sci. USA 89, 10449-53 (1992)). In the experiment, as shown in FIG. 10c, the OD₃₆₀ of the GST protein drastically increased 2 minutes after heat treatment, and most of the protein had aggregated by 3 minutes. In contrast, the GST-synuclein fusion proteins with peptide fragments derived from ATSA did not aggregate at all even 10 minutes after heat treatment. Next, the GST-synuclein fusion proteins with peptide fragments derived from ATSα were qualitatively assayed by monitoring the absorbance at 360 nm while varying the concentration from 0.2 mg/ml to 1.0 mg/ml after heat treatment at 80° C. for 10 minutes. As shown in FIG. 12d, GST-Syn96-140 containing the entire region of ATSα and GST-Syn119-140 containing 22 amino acids of ATSα did not precipitate at all after heat treatment regardless of the concentration, while GST-Syn103-115, GST-Syn114-126 and GST-Syn130-140 containing 11-13 amino acids did not precipitate at all at a low concentration but increasingly aggregated as the concentration rose. It is noted that the aggregation of protein is proportional to the concentration. Thus, it is demonstrated that GST-synuclein fusion proteins with peptide fragments derived from ATSα have heat resistance superior to that of wild type GST and the heat resistance interestingly varies according to the length of ATSα. Therefore, optimum effects can be achieved by suitably selecting the length of ATSα according to the size and property of a target protein.

### Example 13

### Heat Resistance of GST-Synuclein Fusion Protein Containing the C-terminal Acidic Tail Region of β-Synuclein or γ-Synuclein

In addition to α-synuclein, β-synuclein and γ-synuclein, found in human, are proteins constituting the synuclein family, and share a high homology in their amino acid sequences with each other. Particularly, the N-terminal amphipathic region of synuclein strictly conserved among the synuclein family members from the Torpedo to humans. However, the C-terminal acidic tails of the synuclein family members are very diverse in size as well as in sequence (Lavedan C., Genome Research, 8, 871-880 (1998); Lucking C. B. and Brice A. Cell Mol Life Sci, 57, 1894-1908 (2000); Iwai A., Biochem. Biophys. Acta, 1502, 95-109 (2000); and Hashimoto M. and Masliah E. Brain Pathol. 9, 707-720 (1999)). The present inventors examined whether GST-ATSβ and GST-ATSγ fusion proteins containing the acidic tails of β-synuclein (ATSβ) and γ-synuclein (ATSγy), respectively, have heat rewsistance.

GST-ATSβ (SEQ ID NO:86) and GST-ATSγ (SEQ ID NO:87) fusion proteins were prepared by subcloning the ATSβ (residues 85-134) and ATSγ (residues 96-127), respectively, into pGEX vector. The protein coding region of the ATSβ was amplified by PCR with 5′ oligonucleotide primer (SEQ ID NO:36) containing the underlined BamHI restriction site and 3′-oligonucleotide primer (SEQ ID NO:37) containing the underlined XhoI restriction site. The protein coding region of the ATSγ was amplified by PCR with the 5′oligonucleotide primer (SEQ ID NO:38) containing the underlined BamHI restriction site and 3′ oligonucleotide primer (SEQ ID NO:39) containing the underlined EcoRI restriction site.

TABLE 5

| | | |
|---|---|---|
| NO. | Primer | Sequence |

| | | | |
|---|---|---|---|
| 18 | Sense | AGCTAAGGATCCAAGAGGGAGG | (SEQ ID NO:36) |
| | | AATTCC | |
| 19 | Antisense | AAGTAACTCGAGCTACGCCTCT | (SEQ ID NO:37) |
| | | GGCTCATA | |
| 20 | Sense | AAGAATGGATCCCGCAAGGAGG | (SEQ ID NO:38) |
| | | ACTTGA | |
| 21 | Antisense | AATAGCGAATTCCTAGTCTCCC | (SEQ ID NO:39) |
| | | CCACTCT | |

The amplified DNAs were gel purified, digested with appropriate enzymes, then ligated into the pGEX vector which had been digested with appropriate restriction enzymes and gel purified. All expression vectors (pGST-ATSβ and pGST-ATSγ) were verified for their sequences by DNA sequencing. The expression vectors were transformed into the E. coli strain, BL21 (DE3), and the recombinant GST-synuclein fusion proteins (GST-ATSβ and GST-ATSγ) were purified by affinity chromatography using glutathione-Sepharose 4B beads. GST-ATSβ and GST-ATSγfusion proteins were further purified on an FPLC gel-filtration column.

GST-ATSβ and GST-ATSγ fusion proteins were examined for heat-resistance. Each protein suspended in PBS (0.6 mg/ml) was heated in boiling water baths for 10 minutes and cooled in the air. The protein samples were centrifuged at 15,000 rpm for 10 minutes and the supernatants were analyzed on a 12% SDS polyacrylamide gel. The protein bands on the SDS polyacrylamide gel were stained with Coomassie Brillinant blue R250.

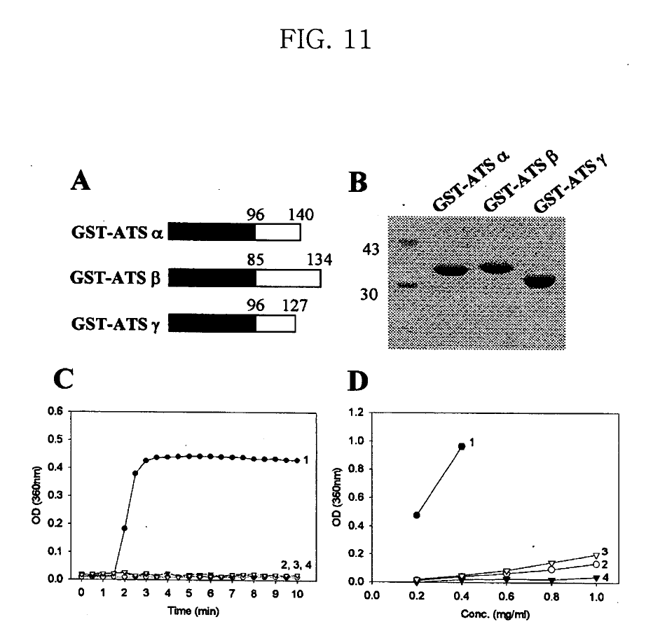

As shown in FIG. 11b, GST-ATSβ and GST-ATSγ as well as GST-ATSα show protein bands after heat treatment, which indicates that they are not precipitated. Therefore, it is demonstrated that the GST-ATSβ and GST-ATSγ fusion proteins have a high heat resistance.

Also, the thermal behaviors of the above GST-ATS fusion proteins were quantitatively assayed by monitoring absorbance at 360 nm according to time while setting the concentration of each protein at 0.2 mg/ml at 65° C. (Lee G. J. and Vierling E., Method Enzymol., 290, 360-65 (1998); and Horwitz J. Proc. Natl. Acad. Sci. USA 89, 10449-53 (1992)). In the experiment, as shown in FIG. lic, the GST protein had almost aggregated after 2 to 3 minutes. In contrast, the above GST-ATS fusion proteins did not aggregate at all even 10 minutes after heat treatment. Next, the above GST-ATS fusion proteins were qualitatively assayed by monitoring the absorbance at 360 nm while varying the concentration from 0.2 mg/ml to 1.0 mg/ml after heat treatment at 80° C. for 10 minutes. As shown in FIG. 11d, the above GST-ATS fusion proteins did not precipitate at all after heat treatment regardless of the concentration, while the GST protein is completely precipitated at a low concentration. Thus, it is demonstrated that in addition to ATSα, the ATSβ and ATSγ are peptides capable of providing heat resistance to other proteins and they can be used in preparation of fusion proteins having resistance to environmental stresses. Also, it is presumed that since the amino acid sequence of synoretin is very similar to that of γ-synuclein, the acidic tail of synoretin may be similarly used.

### Example 14

### Heat-Resistance of GST-Polyglutamate Fusion Proteins Containing the Acidic Tail Composed of Polyglutamate

In the C-terminal acidic tail region of synuclein, a number of negatively charged amino acid residues such as Glu/Asp residues are characteristically scattered therethrough. The present inventors finally examined whether GST-polyglutamate fusion proteins with genuinely negatively charged peptide fragments such as polyglutamate have heat resistance. For this, a series of GST-polyglutamate fusion proteins were constructed by ligating the gene part of polyglutamate into pGEX vector (FIG. 12a). DNAs encoding the part of the polyglutamate peptide were synthesized using an automatic DNA synthesizer (Table 6, SEQ ID NOS:40-43). The oligonucleotides of SEQ ID NOS:40 and 41 were sense and antisense DNAs to synthesize GST-E5 (containing 5 glutamate residues), respectively and the oligonucleotides of SEQ ID NOS:42 and 43 were sense and antisense DNAs to synthesize GST-E10 (containing 10 glutamate residues). The synthesized sense and antisense DNA pairs were annealed and the polyglutamate gene parts were ligated into BamHI and EcoRI restriction sites of the pGEX vectors to construct a series of expression vectors directing GST-polyglutamate fusion proteins. All the expression vectors (pGST-E5 and pGST-E10) were verified for their sequences by DNA sequencing.

TABLE 6

| | | |
|---|---|---|
| NO. | Primer | Sequence |

| | | | |
|---|---|---|---|
| 22 | Sense | GATCCGAAGAAGAAGAAGAA | (SEQ ID NO:40) |
| | | TAA | |
| 23 | Antisense | AATTCTTATTCTTCTTCTTCT | (SEQ ID NO:41) |
| | | TCG | |
| 24 | Sense | GATCCGAAGAAGAAGAAGAAGA | (SEQ ID NO:42) |
| | | AGAAGAAGAAGAATAAG | |
| 25 | Antisense | AATTCTTATTCTTCTTCTTCTT | (SEQ ID NO:43) |
| | | CTTCTTCTTCTTCTTCG | |

The expression vectors pGST-E5 and pGST-E10 were transformed into the E. coli BL21 (DE3). The resulting recombinant proteins were purified by affinity chromatography using glutathione-Sepharose 4B beads. The GST-polyglutamate fusion proteins were further purified on an FPLC gel-filtration column (FIG. 12b) and examined for their heat resistance. Each protein suspended in PBS (0.6 mg/ml) was heated in boiling water baths for 10 minutes and cooled in the air. The protein samples were centrifuged at 15,000 rpm for 10 minutes and the supernatants were analyzed on a 12% SDS polyacrylamide gel. Both GST-E5 and GST-E10 did not show protein bands after heat treatment, which indicates that they had been completely precipitated by heat treatment. Therefore, it is demonstrated that the GST-E5 (SEQ ID NO:88) and GST-E10 (SEQ ID NO:89) do not have heat resistance at such stringent conditions.

Also, the thermal behaviors of the above GST-E5 and GST-E10 fusion proteins were quantitatively assayed by monitoring absorbance at 360 nm according to time while setting the concentration of each protein at 0.2 mg/ml at 65° C. (Lee G. J. and Vierling E., Method Enzymol., 290, 360-65 (1998); and Horwitz J. Proc. Natl. Acad. Sci. USA 89, 10449-53 (1992)). In the experiment, as shown in FIG. 12c, the GST protein were almost aggregated after 2 to 3 minutes and the GST-E5 fusion protein were aggregated in a considerable amount under the same conditions, whereas the GST-E10 fusion protein did not aggregate at all even after heat treatment for 10 minutes at 65° C. Next, the GST-polyglutamate fusion proteins were quantitatively assayed by monitoring the absorbance at 360 nm while varying the concentration from 0.2 mg/ml to 1.0 mg/ml after heat treatment at 80° C. for 10 minutes. As shown in FIG. 12d, the GST protein is completely precipitated at a low concentration and most of the GST-E5 protein was precipitated at a high concentration. In contrast, the GST-E10 protein was partially precipitated after heat treatment under the same conditions and increasingly aggregated as the concentration rose. Thus, it is noted that as the length of polyglutamate increases, the negative charge considerably increases and thereby, aggregation decreases. However, interestingly, it is noted that the polyglutamate tail is considerably less effective to provide heat resistance, as compared to ATS peptides containing the same number of glutamate residues. In fact, GST-Syn130-140 shows heat resistance far superior to GST-E5 containing the same number of glutamate residues and even slightly higher than that of GST-E10 containing two times more glutamate residues (compare FIG. 10d with FIG. 12d). Therefore, it is suggested that the characteristic amino acid sequence of ATS, in addition to the increased solubility of proteins due to the increase of the negative charge, plays an important role in the mechanism, by which fusion proteins with ATS show high resistance to environmental stresses. Also, the present inventors interestingly observed that a fusion protein containing a positively charged peptide such as polyarginine does not show heat resistance at all (data not shown), which supports that the characteristic amino acid sequence of ATS plays a very important role in providing resistance to environmental stresses.

### Example 15

### Preparation of hGH, hGH-Syn119-140 and Syn119-140-hGH Proteins

An expression vector for hGH was constructed by subcloning an hGH gene into a pRSETA expression vector (Invitrogen).

After being isolated from the pituitary gland tissue secreting the human growth hormone, poly(A) mRNA was reacted with an RNA PCR kit (Takara, (AMV) version2.1, Japan) comprising a reverse transcriptase, to obtain double strand CDNA. An hGH-encoding gene was amplified by PCR using a set of the primer (SEQ ID NO:44) containing the underlined NdeI restriction site (5′-GCGCTCGAGCCCATATGTTCCCAACTATACCA-3) and the primer (SEQ ID NO:45) containing the underlined HindIII restriction site (5′-GCGCAAGCTTAAG CTTTTAGAAGCCACAGCTGCC-3). The PCR product was purified by electrophoresis using 1% agarose gel, digested with the restriction enzymes NdeI and HinIII and then ligated into the restriction enzyme sites of the pRETA vector (Pharmacia Biotech, Buckingamshire, UK) to construct the expression vector pRSETA-hGH.

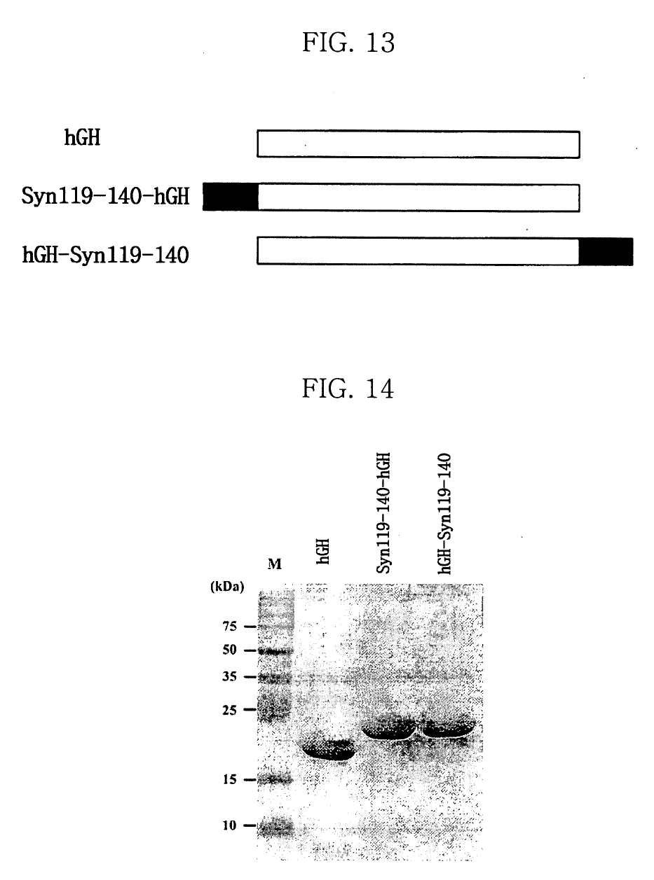

hGH-Syn119-140 and Syn119-140-hGH fusion constructs were prepared by consecutively subcloning an hGH gene and a gene encoding the amino acid residues 119-140 of α-synuclein into the expression vector pRSETA (FIG. 13). In brief, DNAs encoding the amino acid residues 119-140 of ATS awere chemically synthesized (SEQ ID NOS:46, 47, 48, 49). SEQ ID NOS:46 and 47 are DNA sequences for the preparation of the fusion protein SYN119-140-hGH, corresponding to the double strand of ATS, while SEQ ID NOS:48 and 49 are DNA sequences for the preparation of the fusion protein hGH-Syn119-140, corresponding to the double strand of ATS.

For N-terminal fusion, the Syn119-140-encoding cDNAs were digested with NdeI and HindIII and then ligated into a pRSETA vector to construct an ATS-anchored vector (pATS-N). Likewise, for C-terminal fusion, the Syn119-140-encoding cDNAs were digested with BamHI and HindIII and then ligated to a pRSETA vector to construct an Syn119-140-anchored vector (pATS-C). An hGH DNA fragment was excised from the pRSETA-hGH by digestion with BamHI and HindIII, purified by electrophoresis on gel, and then inserted into the same restriction sites of the pATS-N vector to produce a pATS-hGH vector that codes for an Syn119-140-hGH fusion protein. In addition, an hGH DNA fragment was excised from the pRSETA-hGH by digestion with NdeI and BamHI, purified by electrophoresis on gel, and then ligated into the same restriction sites of the pATS-C vector to produce a phGH-ATS vector that codes for an hGH-Syn119-140 fusion protein. All DNA constructs were verified for their sequences by DNA sequencing. hGH- Syn119-140 and Syn119-140-hGH are listed as SEQ ID NOS:90 and 92 for nucleotide sequences and SEQ ID NOS:91 and 93 for amino acid sequences, respectively.

### Example 16

### Expression and Purification of hGH, hGH-Syn119-140 and Syn119-140-hGH Recombinant Proteins

The expression vectors prepared in Example 15 for the expression of hGH, hGH-Syn119-140 and Syn119-140-hGH proteins were introduced into the E. coli strain, BL21 (DE3) pLysS (Invitrogen). The transformed E. coli was cultured in an LB medium containing 0.1 mg/ml ampicillin at 37° C. to an A₆₀₀ of 0.8 and induced with 0.5 mM IPTG, followed by culturing for an additional four hours. The culture was then centrifuged at 10,000 rpm for 10 minutes to harvest cells which were then resuspended in phosphate-buffered saline (PBS, pH 7.4) and disrupted by ultrasonication. All hGH, hGH-Syn119-140 and Syn119-140-hGH proteins were overexpressed at similar expression levels in E. coli, forming inclusion bodies composed of insoluble aggregates of the expressed proteins. The inclusion bodies were recovered by a somewhat modified version of the Kim et al. method (Kim et al., (1997) J. immunol. 159, 3875-3882) and then refolded according to the Patra et al method (Patra et al., (2000) Prot. Exp. Purif. 18, 182-192).

Following the refolding, the proteins were loaded onto a DEAE-Sephacel anion exchange resin packed column which was then washed with 20 mM Tris buffer (pH 8.5) containing 0.1 M NaCl, 5 mM EDTA, 0.4 M urea and 0.02% sodium azide. The samples bound to anion exchange resins were eluted with 100 ml of 20 mM Tris buffer (pH 8.5) and a linear gradient to 0.4 M NaCl in the same buffer (100 ml).

As a last step for protein purification, FPLC (fast protein liquid chromatography) was used in which the eluted proteins were purified on a HiLoad™ 16/60 column and washed with PBS. After being loaded on the column, protein samples were eluted with the buffer for 120 min. Eluted fractions were measured for absorbance at 280 nm to purify the protein uniformly.

hGH-Syn119-140 and Syn119-140-hGH fusion proteins were found to have a refolding efficiency about twice as high as that of the wild type hGH (Table 7), and the same result was also observed by a refolding method using a dilution of a small amount of the sample in a refolding buffer. These results show that the Syn119-140 peptide helps the fused protein refold.

TABLE 7
Purification Yields of hGH, Syn119-140-hGH and hGH-Syn119-140

| | | | Syn119- | Syn119- | hGH- | hGH-Syn119- |
|---|---|---|---|---|---|---|
| | hGH | hGH | 140-hGH | 140-hGH | Syn119-140 | 140 |
| | Total | Total | Total | Total | Total | Total |
| Purification | Protein | Yield | Protein | Yield | Protein | Yield |
| Stages | (mg) | (%) | (mg) | (%) | (mg) | (%) |
| Cell Lysate | 125 | | 115 | | 109 | |
| Inclusion Body | 40 | 100 | 45.1 | 100 | 35.6 | 100 |
| Solubilization | 36 | 90 | 43.8 | 97.1 | 32.2 | 90.4 |
| Ion Exchange | 10.5 | 26.2 | 14.7 | 32.6 | 16.3 | 45.8 |
| Gel filtration | 6.5(±.1) | 16.2 | 11.9(±.34) | 26.4 | 11.3(±.52) | 31.7 |

hGH-Syn119-140 and Syn119-140-hGH were both obtained at yields about twice as high as the wild type hGH (FIG. 14). On SDS-PAGE, bands were visualized at 22 kDa for hGH and at 24 kDa for Syn119-140-hGH and hGH-Syn119-140. The hGH used was identical in size to a standard hGH as measured by SDS-PAGE (data not shown).

MALDI-TOF mass spectrometry showed that the measured molecular weights of hGH, Syn119-140-hGH and hGH-Syn119-140 are coincident with those calculated from the amino acid sequences (Table 8).

TABLE 8
Mw of hGH, Syn119-140-hGH and hGH-Syn119-140 Calculated
theoretically and Measured by MALDI-TOF

| | Mw calculated | Mw Measured |
|---|---|---|
| | (Dalton) | (Dalton) |
| hGH | 22260.3 | 22259.43 |
| Syn119-140-hGH | 24964.9 | 24964.11 |
| hGH-Syn119-140 | 24964.9 | 24957.50 |

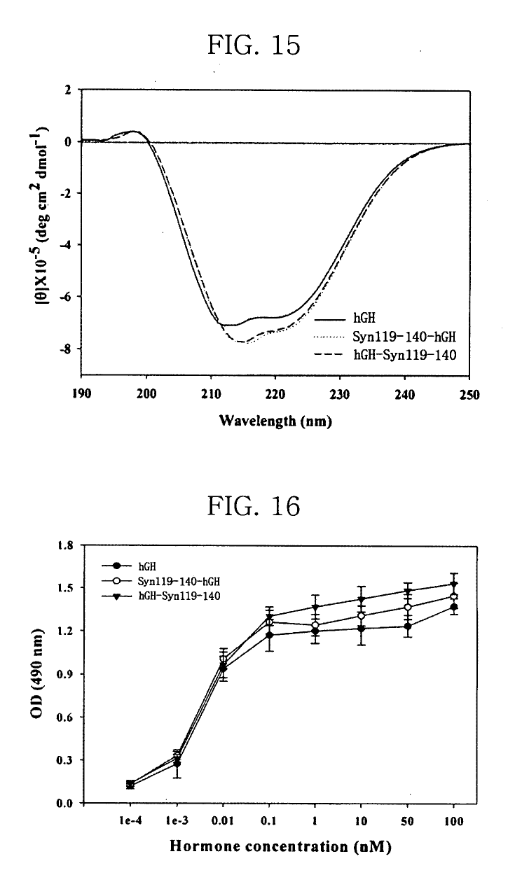

To examine the induced secondary structures of hGH, Syn119-140-hGH and hGH-Syn119-140, CD spectroscopy was conducted in a spectropolarimeter. On the spectra of the hGH proteins, absorbances at 208 and 222 nm, which are characteristic of α-helical protein, were detected (FIG. 15, solid line: hGH, dashed line: hGH-Syn119-140, dotted line: Syn119-140-hGH). Also, the proteins were found to have a conformation very similar to that of the wild type. These spectral data shows that the fusion proteins have an accurately refolded structure, which was further verified through a biological activity test.

### Example 17

### Measurement of Biological Activity of Proteins of hGH, hGH-Syn119-140 and Syn119-140-hGH

Biological activities of hGH, hGH-Syn119-140 and Syn119-140-hGH proteins were quantitatively analyzed by Nb2 cell proliferation assay (Tanaka et al., (1980) J. Clin. Endoclinol. Metab. 51, 1058-1063; Dattani et al., (1995) G120R, J. Biol. Chem. 270, 9222-9226; Peterson et al., (1997) J. Biol. Chem. 272, 21444-21448), and verified by detecting a phosphorylated form of STAT-5 (Friedrichsen et al., (2001) Mol Endocrinol. 15, 136-148; Graichen et al., (2003) J. Biol. Chem. 278, 6346-6354).

Nb2-11 rat lymphoma cells (Tanaka et al., (1980), J. Clin. Endoclinol. Metab. 51, 1058-1063) were purchased from ECACC (European Collection of Cell Culture). All the glass tools and instruments, media and distilled water which were used for animal cell culture were sterilized before use, or were sterile products. An RPMI1640 medium (GibcoBRL, Cat #: 31800-022) was dissolved in deionized water, added with 0.37% disodium carbonate, titrated to pH 7.2 using HCl, and sterilized by passing through a filter having a pore size of 0.22 μm. Cells were cultured in the sterilized medium, supplemented with 10% horse serum, 2mM mercaptoethanol, 50 units/ml penicillin, 50 μg/ml streptomycin and 2×10⁻³ M L-glutamine, while the medium was completely refreshed every two or three days. When reaching 80-90% confluence on the surface of the culture plate, the cells were sub-cultured. 2×10⁴ cells were loaded into each well of 96 well plates (Costar, Cambridge, Mass.) in triplicate. The each of the hGH, Syn119-140-hGH and hGH-Syn119-140 proteins were diluted to 1×10⁻⁴ nM to 100 nM with the same medium and were added to the wells, and then cells were stimulated for 48 hours. The cell culture volume in each well was fixed at 100 μl . Cell viability was determined by an MTS assay, which is based on the conversion of MTS by mitochondrial dehydrogenase to a brown product, as measured at an absorbance of 490 nm. 80 μl of MTS was added to 100 μl of the medium containing the Nb2 cells cultured in the 96-well plates, followed by incubation at 37° C. for three hours in a 5% carbon dioxide incubator.

In the presence of hGH-Syn119-140 or Syn119-140-hGH, Nb2 cells followed proliferation patterns similar to that observed in the presence of hGH, as seen in FIG. 16.

### Example 18

### Immunophoretic and Western Blotting Assay of hGH, hGH-Syn119-140 and Syn119-140-hGH

Cells treated or not treated with hGH samples according to the Wang et al. method (Wang et al., (1994), Proc. Natl. Acad. Sci. USA 91, 1391-1395) were disrupted to prepare whole cell lysates which were then subjected to Western blotting using a monoclonal antibody against a tyrosine phosphorate form of STAT-5 according to the protocol provided from the manufacturer (Transduction Laboratories), which is based on the principle that when hGH binds to GH receptors on a cell surface, intracellular Jak2 tyrosine kinase is activated and then gathers and phosphorylates STAT-5 at the tyrosine position.

The Nb2 cell strain was stimulated with I nM of hGH, Syn119-140-hGH or hGH-Syn119-140 for 15 min or 30 min. After pipetting for cell separation, the culture was centrifuged at 15,000 rpm for 5 min to harvest cells as a pellet. This cell pellet was added to 100 μl of a lysis buffer and allowed to stand for 30 min in ice, followed by centrifugation at 20,000 rpm for 20 min. The supernatant was electrophoresed on a 12% SDS PAGE gel and the separated proteins on the gel were transferred onto a PVDF membrane with the aid of a gel-membrane transferring kit in the presence of 500 mA for 90 min. Thereafter, the membrane was treated for one hour with a blocking buffer containing 3% non-fat dry milk to block the background signals attributed to non-specific binding. The membrane was incubated, along with a monoclonal anti-stat5 primary antibody (1:500 diluted), for two hours at room temperature in a washing buffer containing 3% non-fat dry milk, and then washed three times for 10 min with the washing buffer. After another incubation with horse radish peroxidase-conjugated secondary antibody (diluted 1:1,000) for one hour at room temperature in a washing buffer containing 3% non-fat dry milk, the membrane was washed four times for 10 min with the washing buffer. Treatment with a mixture of DAP (20 pg) and hydrogen peroxide (30 pl) made the bands formed on the membrane visible.

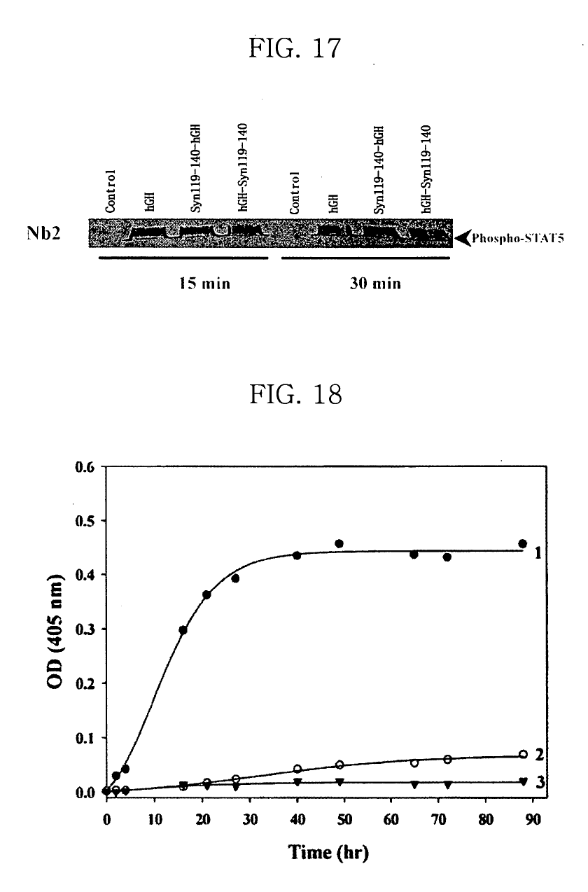

In Nb2 cells, the hGH fusion proteins were found to effectively phosphorylate STAT-5 (FIG. 17). These results reveal that the fusion proteins hGH-Syn119-140 and Syn119-140-hGH retain the same biological functionality as that of the wild type hGH.

### Example 19

### Assay for Shaking-Induced Aggregation of hGH, hGH-Syn119-140 and Syn119-140-hGH

hGH, Syn119-140-hGH and hGH-Syn119-140, all prepared in Example 15, were observed for aggregation induced by shaking over time.

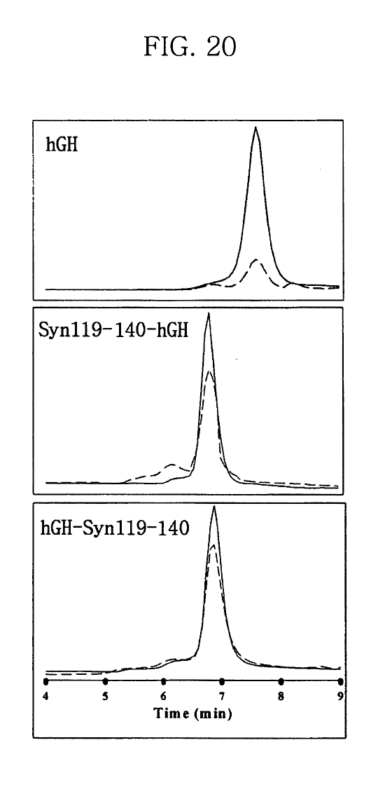

A suspension of 1 mg/ml of each of the proteins in PBS buffer (pH 7.4) was passed through a 0.2 μm syringe filter to remove any protein masses that might act as protein aggregation seeds. Each of the filtered protein suspensions was continuously shaken at room temperature using an orbital shaker (Superteck, Seolin Science Korea) rotating at 150 rpm. The shaking-induced aggregation was quantitatively analyzed by determining the turbidity based on the measurements of absorbance at 405 nm every hour (FIG. 18). Additionally, while being shaken, the proteins were sampled every 24 hours. After the samples were centrifuged to remove insoluble aggregates, the supernatants were loaded onto columns of HPLC gel filtration chromatography and washed for 15 min with PBS buffer. Protein aggregation was analyzed on the basis of the absorbance measured at 280 nm (FIG. 20).

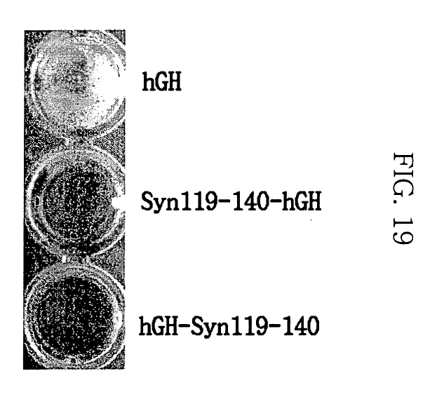

Shaking is a stress which occurs between protein solutions and air in the production, delivery and treatment of therapeutic proteins. As seen in FIG. 18, none of the fusion proteins hGH-Syn119-140 and Syn119-140-hGH formed aggregation even after shaking for 90 hours, whereas the wild type hGH quickly aggregated within a few hours of shaking. Further, the same results were obtained from tests in which higher concentrations of the proteins were used (data not shown).

Whereas the shaking-induced aggregates of hGH could be seen with the naked eye, the solutions of hGH-Syn119-140 or Syn119-140-hGH remained clear after shaking (FIG. 19).

It is clearly apparent from these results that the Syn119-140 peptide fused to hGH can effectively protect the hGH from shaking-induced aggregation.

### Example 20

### Assay for Freezing/Thawing-Induced Aggregation of hGH, hGH-Syn119-140 and Syn119-140-hGH

Stability against repeated freezing/thawing stress was examined in hGH, hGH- Syn119-140 and Syn119-140-hGH. Each protein was suspended at a concentration of 1 mg/ml in PBS buffer (pH 7.4). The protein samples were induced to aggregate by repeating a cycle of freezing in liquefied nitrogen and thawing in a water bath of 37° C. The extent of the protein aggregation was monitored by measuring absorbance at 405 nm every five freezing/thawing cycles. After 15 cycles, centrifugation was conducted to remove insoluble aggregates. The supernatants were loaded on columns of HPLC gel filtration chromatography and washed for 15 min with PBS buffer. Protein aggregation was analyzed on the basis of the absorbance measured at 280 nm.

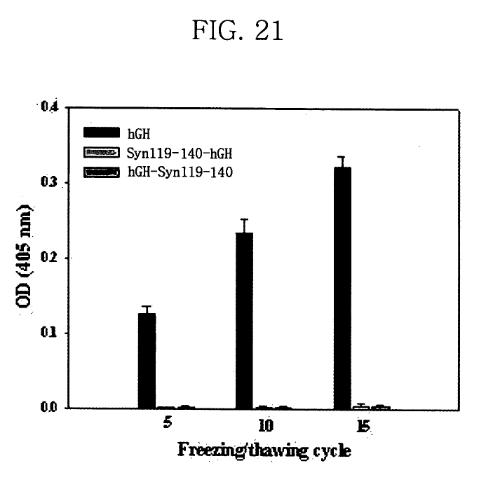

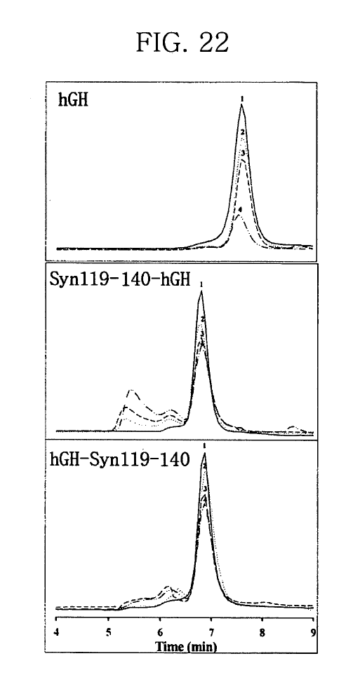

The wild-type hGH readily aggregates as the number of freezing/thawing cycles increases (FIG. 21). As measured by HPLC gel filtration chromatography, the wild type hGH was found to aggregate to a significant extent from the fifth repeated cycle and almost completely after 15 repeated cycles (FIG. 22 upper top). In contrast, HPLC gel filtration chromatography results show that both hGH-Syn119-140 and Syn119-140-hGH, which underwent the same stress as in the hGH, are highly resistant to the environmental stress of freezing/thawing (FIG. 22, middle and bottom). As seen in FIG. 22, the Syn119-140 fusion proteins exist, for the most part, as monomers though the content of oligomer is increased a little as the number of repeated freezing/thawing cycles increases. Particularly, Syn119-140-hGH was found to be more stable to freezing/thawing stress than was hGH-Syn119-140.

### Example 21

### Assay for pH-Induced Aggregation of hGH, hGH-Syn119-140 and Syn119-140-hGH

The pH-induced aggregation of hGH, Syn119-140-hGH and hGH-Syn119-140 was quantitatively analyzed by determining the turbidity based on the measurements of absorbance at 405 nm according to pH. Each protein was diluted to a final concentration of 0.2 mg/ml in buffers with different pH values. The buffers used were mixtures of 0.1 M citrate, succinate, Tris, HEPES, acetate and glycine, which were adjusted to pH 3-12. The protein solutions diluted in the buffers were incubated for 1 hour at 25° C. and their apparent absorbance were measured in a Beckman spectrophotometer.

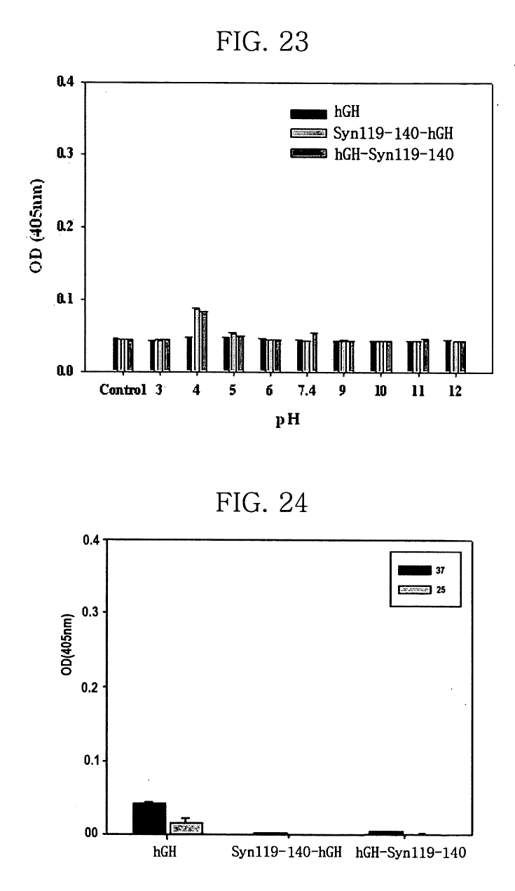

In order to exclude the effect of salts of the buffer on protein aggregation as much as possible, the mixed buffers were used in the whole pH range. As seen in FIG. 23, no aggregates of hGH were found in the whole pH range. These results are coincident with the previous reports that hGH does not aggregate under acidic or alkali conditions of low salt concentrations.

### Example 22

### Assay for storage Stability of hGH, hGH-Syn119-140 and Syn119-140-hGH

hGH, Syn119-140-hGH and hGH-Syn119-140 were assayed for storage stability by conducting SDS-PAGE and measuring turbidity while the proteins were stored at temperatures higher than a typical storage temperature.

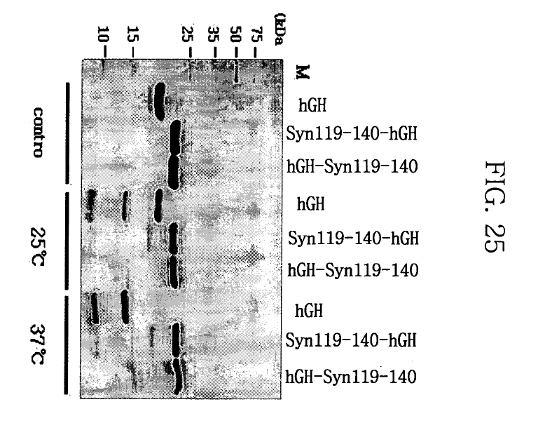

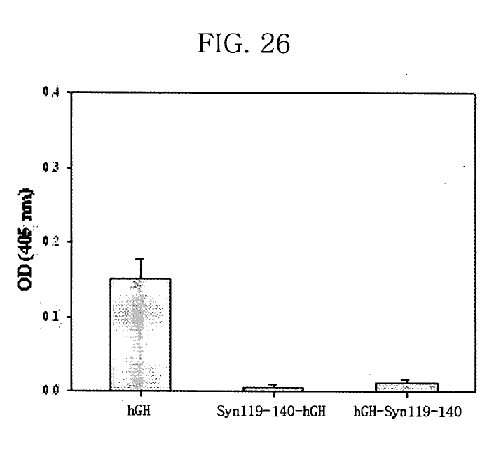

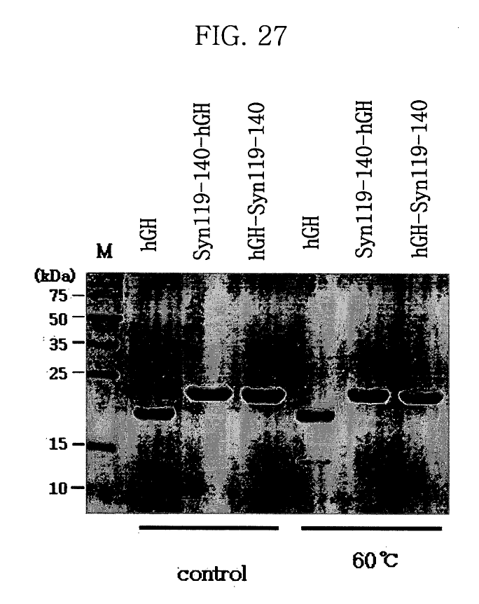

Each protein was suspended at a concentration of 1 mg/ml in PBS buffer (pH 7.4). At 25° C., 37° C. and 60° C., the protein suspension samples were observed for storage stability. The aggregation of each protein sample was determined by measuring absorbance at 405 nm according to time (FIGS. 24 and 26), after which SDS-PAGE was conducted to confirm the results (FIGS. 25 and 27).

As seen in FIG. 24, no proteins were observed to aggregate at 25° C. and 37° C. After storage for 30 days at 25° C. and 30° C., hGH was mostly denatured while hGH-Syn119-140 and Syn119-140-hGH maintained their original states (FIG. 25). In the 60° C. test, hGH was denatured to a significant degree after three days storage (FIG. 26). In contrast, most of the Syn119-140 fusion proteins remained soluble in this period. SDS-PAGE shows that after storage for three days at 60° C., hGH was mostly denatured but most of the Syn119-140 fusion proteins retained their original sizes (FIG. 27).

From these results, it is inferred that the Syn119-140 peptide can increase the storage stability of the protein fused thereto in solutions.

### Example 23

### Assay for Heat-Induced Aggregation of hGH, hGH-Syn119-140 and Syn119-140-hGH

To qualitatively assay hGH, Syn119-140-hGH and hGH-Syn119-140 for heat-induced aggregation, heat treatment was followed by SDS-PAGE.

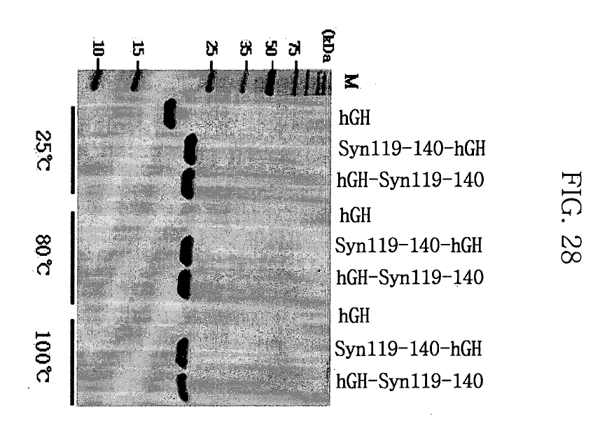

Each protein was suspended at a concentration of 1 mg/ml in PBS buffer (pH 7.4). The suspensions were treated on a hot plate at 100° C. for 10 min and allowed to stand at room temperature for 10 min. After the thermally treated protein samples were centrifuged at 15,000 rpm for 10 min, the supernatants were analyzed by 15% SDS-PAGE. As seen in FIG. 28, the fusion proteins hGH-Syn119-140 and Syn 119-140-hGH did not aggregate at all even after heat treatment at 100° C. for 10 min, but complete aggregation was found in hGH.

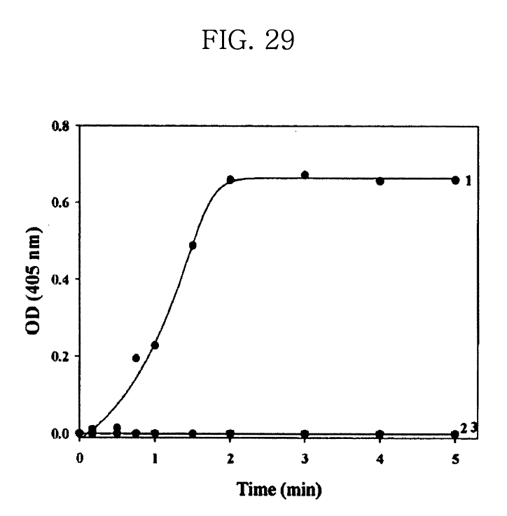

For the quantitative analysis of heat-induced aggregation, the apparent absorbance at 405 nm of hGH, Syn119-140-hGH and hGH-Syn119-140 was measured after treatment at 80° C. according to time. Each of the proteins was diluted to a final concentration of 0.5 mg/ml in PBS buffer and put into an absorption spectrometer cuvette, and the apparent absorbance was measured in a Beckman spectrophotometer equipped with an automatic temperature controller. As seen in FIG. 29, hGH almost aggregated within 2-3 min while hGH-Syn119-140 and Syn119-140-hGH did not aggregate even 10 min after heat treatment.

A suspension of 1 mg/ml of each of the proteins in PBS buffer (pH 7.4) was treated for 10 min each at 25° C., 65° C., 70° C., 75° C. and 80° C., after which insoluble aggregates were removed by centrifugation, followed by analysis with HPLC gel filtration chromatography (FIG. 30). The supernatant samples were loaded onto columns and washed with PBS buffer for 15 min during which chromatographs were obtained by measuring the absorbance at 280 nm. HPLC gel filtration chromatography analysis reveals that the heat-treated hGH sample aggregates at 80° C. and completely loses the monomer conformation and functionality (FIG. 30, top). However, the hGH-Syn119-140 and Syn119-140-hGH fusion proteins, even if the contents of oligomers were increased, did not aggregate (FIG. 30, middle and bottom).

These results show that the Syn119-140 peptide confers heat resistance and thermosolubility to the hGH fused thereto.

### Example 24

### Measurement of 2′ Structural Change by CD Spectroscopy of hGH, hGH-Syn119-140 and Syn119-140-hGH

To analyze the secondary structural change of hGH, Syn119-140-hGH and hGH-Syn119-140 with temperature increase, CD spectra of the proteins were measured using a Jasco-J810 spectropolarimeter (Jasco, Japan) equipped with a temperature control system in a continuous mode.

The far-UV CD measurements were carried out over the wavelength range of 190 to 250 nm with a 0.5 nm bandwidth, a one second response time and a 10 nm/minute scan speed at 25° C. The spectra shown are an average of five scans that were corrected by subtraction of the buffer signal. The CD data were expressed in terms of the mean residue ellipticity, [0] (deg.cm2.dmol-1). Thermal denaturation experiments were performed using a heating rate of 1° C./min and a response time of 1 second. Purified protein preparations at a protein concentration of 0.5 mg/ml in a cuvette with a path length of 0.1 cm were used. The thermal scan data were collected from 25 to 100° C. The CD spectra were measured every 0.5° C. at a wavelength of 222 nm.

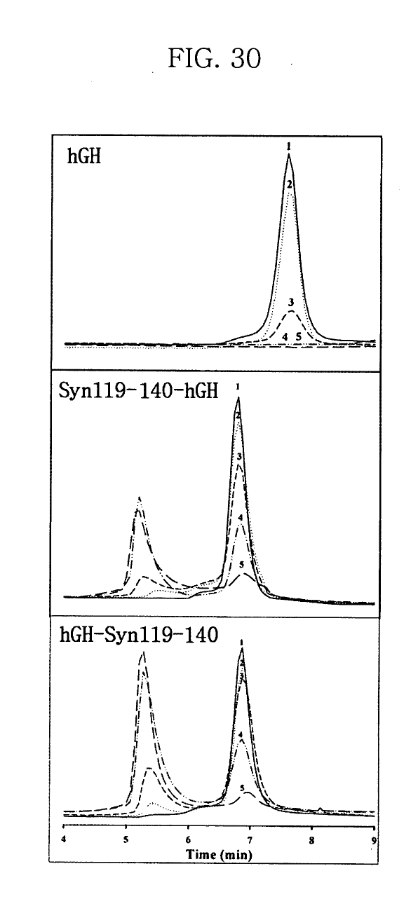

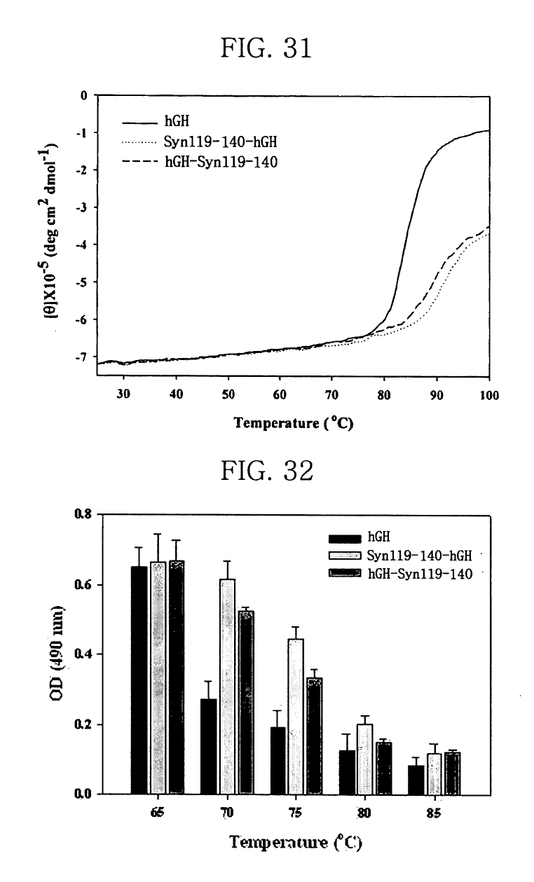

Paricularly, while temperatures were changed, the heat-induced unfolding of the proteins was measured at 222 nm in order to compare their stability to heat (FIG. 31). As reported in previous literature (Filikov, A. V., Hayes, R. J., Luo, P., Stark, D. M., Chan, C., Kundu, A., Dahiyat, B. I. (2002) Protein Sci. 11, 1452-1461), hGH started to unfold at 78° C., and showed a melting temperature (Tm) of 80° C. However, hGH-Syn119-140 and Syn119-140-hGH were found to unfold at higher temperatures (FIG. 31, represented by dashed line and dotted line, respectively). Upon heat treatment, hGH-Syn119-140 started to unfold at around 83° C., with a Tm of 87° C. The unfolding of Syn119-140-hGH by heat treatment started at around 85° C. and its Tm was measured at 90° C. The Tm values given, although not accurate thermodynamic values, demonstrate that the Syn119-140 peptide significantly improves the thermal stability of the hGH fused thereto. In addition, the biological activities of the hGH samples treated at 65° C., 70° C., 75° C., 80° C. and 85° C. testify that the fusion proteins hGH-Syn119-140 and Syn119-140-hGH are far superior in heat stability to the wild type hGH (FIG. 32).

### Example 25

### Effect of Syn119-140 peptide fusion on the pharmacokinetics of hGH

In vivocomparison of pharmacokinetics among hGH, Syn119-140-hGH (ATS linked to the N-terminus of hGH) and hGH-Syn119-140 (ATS linked to the C-terminus of hGH) was conducted in rats. For this comparison, 12 female Sprague-Dawley rats (280±10 g) were randomly divided into four groups. The same molar concentration of hGH (96mg/kg), Syn119-140-hGH (110 mg/kg) and hGH-Syn119-140 (110 mg/kg) was subcutaneously injected once into the rats according to group, followed by measuring blood hGH levels. Blood samples were taken every hour after the injection, and diluted 1:1 with an EDTA solution in PBS before storage. After being allowed to stand in ice for one hour, the sample dilutions were centrifuged to separate plasma which was then stored at −20° C. until use in the analysis of blood hGH levels. An ELISA-kit for detecting hGH, e.g. a kit commercially available from Roche, was used for the analysis of samples.

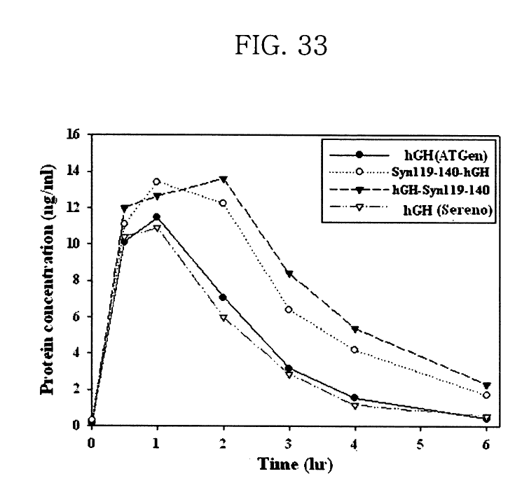

The results are depicted in FIG. 33. As seen, the blood levels of the wild-type hGH proteins (commercially available from ATGgen and Sereno) remained high until one hour after the injection, and then sharply decreased. In contrast, the fusion proteins of Syn119-140 and hGH maintained high blood levels until two hours after the injection and then, decreased in blood level more gradually than the wild types. As measured on the basis of the graph of FIG. 33, the hGH proteins, whether synthesized by the present inventors or commercially available from Sereno, were both found to have a half life of two hours in blood, whereas the half life in blood of the fusion proteins of hGH and Syn119-140 was four hours, double that of the hGH proteins. These results exhibit that the hGH proteins fused to Syn119-140 are much more stable in vivoas well as in vitro than the wild type proteins. The longer half life periods in blood of the fusion proteins of Syn119-140 and hGHs than those of the wild type, in our knowledge, are probably attributed to the fact that the Syn119-140 peptide would not only confer resistance to stresses but also protect the attack of serum proteases.

### Example 26

### Resistance of the Fusion Proteins (hGH-ATSw and hGH-ATSp) of hGH and Representative Peptides Containing Whole or Fragment of Syn119-140 Peptide

Two fusion proteins (hGH-ATSw and hGH-ATSp) containing whole or fragment of Syn119-140 peptide or fragment of Syn119-140 peptide, respectively, plus hGH were examined for their resistance to environmental stresses.

hGH-ATSw and hGH-ATSp fusion protein constructs were prepared by subcloning, instead of a gene encoding the Syn119-140 (amino acid residues 119-140 of α-synuclein), a gene encoding the Syn119-140 peptide plus five amino acid residues (ATSW, 27 amino acid residues length) or a gene encoding fragment of the Syn119-140 peptide (ATSP, 17 amino acid residues length). In brief, two DNA fragments respectively encoding ATSw and ATSp were amplified by PCR using a set of two primers having BamHI and EcoRI restriction sites, respectively. After being cut with the restriction enzymes BamHI and EcoRI, each of the PCR products was ligated to the hGH gene of hGH-Syn119-140 fusion protein coding construct which had already been digested with the same restriction enzymes, so as to produce DNA constructs coding for hGH-ATSw and hGH-ATSp fusion proteins. All DNA constructs were verified for their sequences by DNA sequencing.

TABLE 9

Primers for use in the preparation of expression
vectors for fusion proteins of hGH, hGH-ATSw and
hGH-ATSp

| | | |
|---|---|---|
| Proteins | Primers | Primer Sequences |

| | | | |
|---|---|---|---|
| hGH-ATSw | ATSw-BamHI | 5′-GCA ACT GGA | (SEQ ID NO:50) |
| | | TCC GAA GAT ATG | |
| | | CCT GTG | |
| hGH-ATSw | ATSw-EcoRI | 5′-ACT GCC GAA | (SEQ ID NO:51) |
| | | TTC TTA GGC TTC | |
| | | AGG TTC | |
| hGH-ATSp | ATSp-BamHI | 5′-GCA ACT GGA | (SEQ ID NO:52) |
| | | TCC GAT CCT GAC | |
| | | AAT GAG | |
| hGH-ATSp | ATSp-EcoRI | 5′-ACT GCC GAA | (SEQ ID NO:53) |
| | | TTC TTA GTC TTG | |
| | | ATA CCC | |

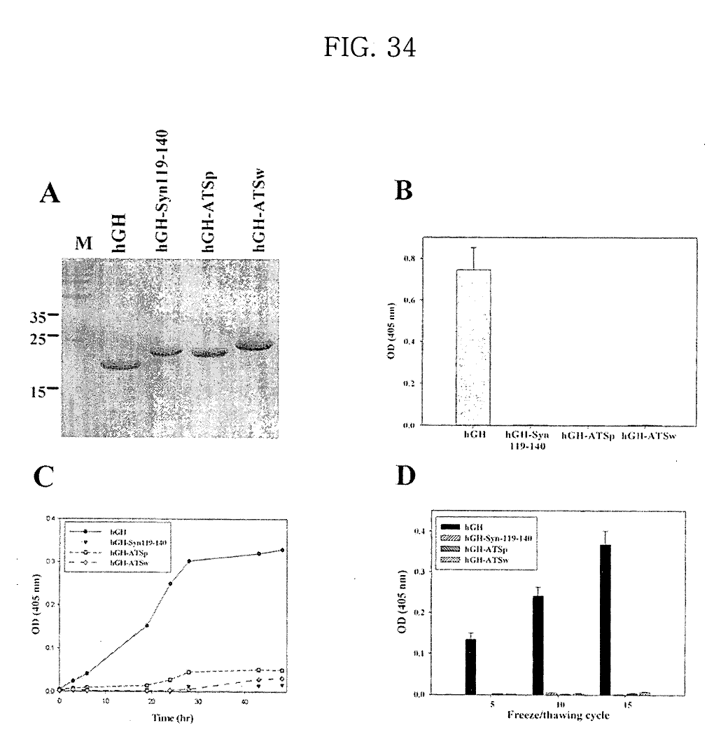

Both hGH-ATSw(SEQ ID NO:94) and hGH-ATSp(SEQ ID NO:95) fusion proteins were overexpressed at similar expression levels in E coli, forming inclusion bodies composed of insoluble aggregates of the expressed proteins. The inclusion bodies were recovered by a somewhat modified version of the Kim et al. method (Kim, J., Chwae, Y. J., Kim, M. Y., Choi, I. H., Park, J. H., Kim, S. J. (1997) J. Immunol. 159, 3875-3882). The refolding of the proteins was achieved by a somewhat modified version of the Patra et al's alkali method (Patra, A. K., Mukhopadhyay, R., Mukhija, R., Krishnan, A., Garg, L. C., Panda, A. K. (2000) Prot. Exp. Purif. 18, 182-192), followed by conducting a column chromatography to purify the proteins. The fusion proteins hGH-ATSw and hGH-ATSp were found to be 23-24 kDa in size as analyzed by SDS-PAGE (FIG. 34a). MALDI-TOF mass spectrometry showed that the measured molecular weights of ATS fusion proteins are not different from those calculated from the amino acid sequences (data not shown).

Amino acid sequences of the ATS peptides tested are listed in Table 10, below.

TABLE 10
Amino acid sequences of ATS, ATSw and ATSp

| | |
|---|---|
| Amino Acid Sequence | Features |

| | | |
|---|---|---|
| Syn119-140 | DPDNEAYEMPSEEGYQDY | Syn119-140 |
| | EPEA | (SEQ ID NO:7) |
| ATSw | EDMPVDPDNEAYEMPSEEGY | 5 a.a. added to |
| | QDYEPEA | Syn119-140 |
| | | (SEQ ID NO:9) |
| ATSp | DPDNEAYEMPSEEGYQD | 5 a.a deleted from |
| | | Syn119-140 |
| | | (SEQ ID NO:10) |

The fusion proteins (hGH-ATSw and hGH-ATSp) of hGH and two representative peptides containing the whole region or fragment of the Syn119-140 peptide, along with the fusion protein hGH-Syn119-140 and the wild type hGH, were analyzed for resistance to environmental stresses.

First, they were compared with regard to the aggregation behavior induced by heat. A suspension of each of the protein samples (I mg/ml) in PBS buffer was treated on a hot plate at 100° C. for 10 min, allowed to stand at room temperature for 10 min, and measured for apparent absorbance at 405 nm through which the heat-induced aggregation levels of the proteins were determined. As seen in FIG. 34b, hGH completely aggregated whereas all of the hGH-ATS fusion proteins seldom aggregated even after heat treatment at 100° C. for 10 min. The heat resistance of hGH-ATSp is somewhat poorer than that of hGH-ATS and hGH-ATSw, but much higher than that of the wild type hGH (FIG. 34b).

Next, the fusion proteins hGH-ATSw, hGH-ATSp and hGH-ATS, and the wild type hGH were examined for shaking-induced aggregation. A suspension of 1 mg/ml of each of the proteins in PBS buffer (pH 7.4) was passed through a 0.2 μm syringe filter to remove any protein masses that might act as protein aggregation seeds. Each of the filtered protein suspensions was continuously shaken at room temperature using an orbital shaker (Superteck, Seolin Science Korea) rotating at 150 rpm. The shaking-induced aggregation was quantitatively analyzed by determining the turbidity based on the measurements of absorbance at 405 nm every hour. As seen in FIG. 34c, none of hGH-ATS, hGH-ATSw or hGH-ATSp form aggregates even after shaking for 50 hours, whereas the wild type hGH quickly aggregated within a few hours of shaking.

Finally, stability against repeated freezing/thawing stress was examined in hGH, hGH-ATS, hGH-ATS2 and hGH-ATSp. Each protein sample was suspended at a concentration of 1 mg/ml in PBS buffer (pH 7.4). Protein aggregation was achieved by repeating a cycle of freezing in liquefied nitrogen and thawing in a water bath of 37° C. The extent of the protein aggregation was monitored by measuring absorbance at 405 nm every five freezing/thawing cycles. The wild-type hGH readily aggregates as the number of the freezing/thawing cycles increases (FIG. 34d). By contrast, all of hGH-ATS, hGH-ATSw and hGH-STSp were found to have great stability against repeated freezing/thawing stresses (FIG. 34d).

Taken together, the results obtained above exhibit that, like the intact Syn119-140 peptide, both the two representative ATS peptides, containing the whole region or fragment region of the ATS peptide, respectively, have the ability to confer resistance to environmental stresses to fusion partner proteins fused thereto, thereby guaranteeing the activity of the fusion proteins in vivo as well as in vitro. When account is taken of the results, any synthetic peptide containing whole or fragment of ATS is expected to have similar functionality.

### Example 27

### Effect of Point Mutant Syn119-140 Peptide Fusion on Resistance of hGH Resistance to Stress

For the preparation of an expression vector of point mutant hGH-Syn119-140, site-directed mutagenesis was applied to an hGH-Syn119-140 fusion DNA construct. In brief, a mutant hGH-Syn119-140 fusion DNA construct is prepared by PCR using a set of a primer having one or two bases mutated at a predetermined site and a complimentary primer (Table 11). After being digested with the restriction enzyme Dpn I, the PCR product was anchored in an expression vector which was then transformed into E coli XL10 gold. Mutant sequences of all DNA constructs were verified by DNA sequencing.

TABLE 11

Primer sequences for the preparation of expression
vector containing point mutant hGH-Syn119-140

| | |
|---|---|
| mutant | Sequence |

| | | | |
|---|---|---|---|
| E123A | S | 5′-CCT GAC AAT GCG GCT TAT | (SEQ ID NO:54) |
| | | GAA ATG | |
| E123A | AS | 5′-CAT TTC ATA AGC CGC ATT | (SEQ ID NO:55) |
| | | GTC AGG | |
| Y133A | S | 5′-GAG GAA GGG GCT CAA GAC | (SEQ ID NO:56) |
| | | TAC | |
| Y133A | AS | 5′-GTA GTC TTG AGC CCC TTC | (SEQ ID NO:57) |
| | | CTC | |
| A124E | S | 5′-GAC AAT GAG GAA TAT GAA | (SEQ ID NO:58) |
| | | ATG | |
| A124E | AS | 5′-CAT TTC ATA TTC CTC ATT | (SEQ ID NO:59) |
| | | GTC | |
| N122V | S | 5′-GAT CCT GAC GTG GAG GCT | (SEQ ID NO:60) |
| | | TAT G | |
| N122V | AS | 5′-C ATA AGC CTC CAC GTC | (SEQ ID NO:61) |
| | | AGG ATC | |
| M127S | S | 5′-GCT TAT GAA AGC CCT TCT | (SEQ ID NO:62) |
| | | GAG | |
| M127S | AS | 5′-CTC AGA AGG GCT TTC ATA | (SEQ ID NO:63) |
| | | AGC | |
| A140S | S | 5′-GAA CCT GAA AGC GGA TCC | (SEQ ID NO:64) |
| | | TTC C | |
| A140S | AS | 5′-G GAA GGA TCC GCT TTC | (SEQ ID NO:65) |
| | | AGG TTC | |

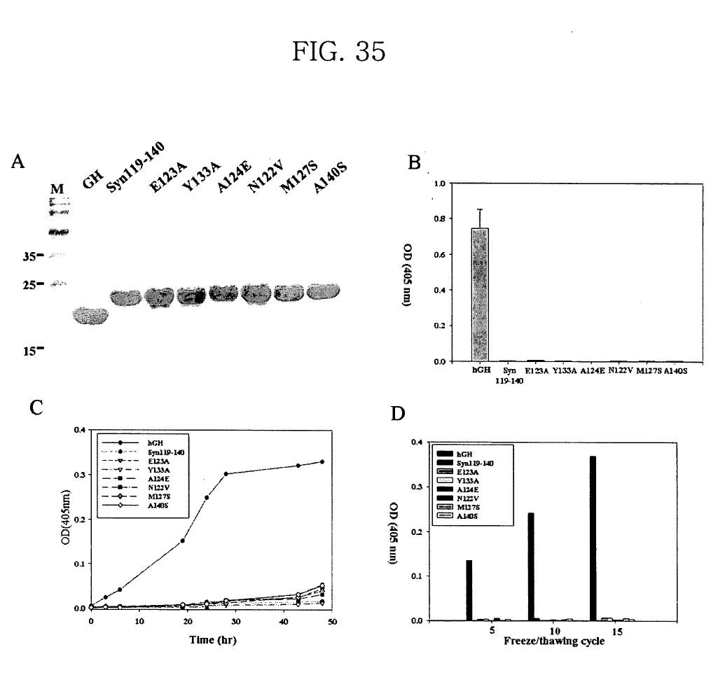

All of the fusion proteins of hGH and six point mutants of Syn119-140, including A124E (Table 11), were overexpressed at similar expression levels in E coli, forming inclusion bodies composed of insoluble aggregates of the expressed proteins. The inclusion bodies were recovered by a somewhat modified version of the Kim. Et al method (Kim, J., Chwae, Y. J., Kim, M. Y., Choi, I. H., Park, J. H., Kim, S. J. (1997) J. Immunol. 159, 3875-3882). The refolding of the proteins was achieved by a somewhat modified version of Patra et al's alkali method (Patra, A. K., Mukhopadhyay, R., Mukhija, R., Krishnan, A., Garg, L. C., Panda, A. K. (2000) Prot. Exp. Purif. 18, 182-192), followed by conducting a column chromatography to purify the proteins. All of the Syn119-140 point mutant fusion proteins were found to have a size of 24 kDa as analyzed by SDS-PAGE (FIG. 35a). MALDI-TOF mass spectrometry showed that the measured molecular weights of Syn119-140 point mutant fusion proteins are identical to those calculated from the amino acid sequences (data not shown).

Amino acid sequences of the point mutant ATS peptides in the six hGH-ATS fusion proteins are listed in Table 12, below. To examine whether an increase or decrease in the number of negatively charged residues of the ATS peptide influences the resistance of the fusion protein to stresses, the point mutants E132A and A124E were prepared. Examination was made of the effect of an increase or decrease in the number of the hydrophobic residues of the ATS peptide on the resistance of the fusion protein to stresses, using the point mutants Y133A and N122V. The influence of a change in the residues that are not conserved in the synuclein family on the resistance of the fusion protein to stresses was examined with the point mutants M127S and A140S.

TABLE 12
Syn119-140 region amino acid sequences in point mutant
hGH-Syn119-140

Sequence

| | | |
|---|---|---|
| Syn119-140 | DPDNEAYEMPSEEGYQDYEPEA | (SEQ ID NO:7) |
| | (a.a. res. 119-140 of α-synuclein) | |
| E123A | DPDNAAYEMPSEEGYQDYEPEA | (SEQ ID NO:11) |
| | (one (−) charge decreased) | |
| Y133A | DPDNEAYEMPSEEGAQDYEPEA | (SEQ ID NO:12) |
| | (one hydrophobic res. decreased) | |
| A124E | DPDNEEYEMPSEEGYQDYEPEA | (SEQ ID NO:13) |
| | (one (−) charge increased) | |
| N122V | DPDVEAYEMPSEEGYQDYEPEA | (SEQ ID NO:14) |
| | (one hydrophobic res. increased) | |
| M127S | DPDNEAYESPSEEGYQDYEPEA | (SEQ ID NO:15) |
| | (one non-conserved res. substituted) | |
| A140S | DPDNEAYEMPSEEGYQDYEPES | (SEQ ID NO:16) |
| | (one non-conserved res. substituted) | |

hGH-Syn(E123A) (SEQ ID NO:96), hGH-Syn(Y133A) (SEQ ID NO:97), hGH-Syn(A124E) (SEQ ID NO:98), hGH-Syn(N122V) (SEQ ID NO:99), hGH-Syn(M127S) (SEQ ID NO:100) and hGH-Syn(A140S) (SEQ ID NO:101), along with the fusion protein hGH-Syn119-140 and the wild type hGH, were analyzed for resistance to environmental stresses.

First, the heat-induced aggregation of the proteins was analyzed. A suspension of each of the protein samples (1 mg/ml) in PBS buffer was treated on a hot plate at 100° C.for 10 min, allowed to stand at room temperature for 10 min, and measured for apparent absorbance at 405 nm through which the heat-induced aggregation levels of the proteins were determined. As seen in FIG. 35b, hGH completely aggregated whereas all of hGH-Syn119-140 and the six point mutant hGH-Syn9-140 fusion proteins seldom aggregated even after heat treatment at 100° C. for 10 min.

Next, the point mutant hGH-Syn119-140 fusion proteins were examined for shaking-induced aggregation. A suspension of 1 mg/ml of each of the proteins in PBS buffer (pH 7.4) was allowed to pass through a 0.2 pm syringe filter to remove any protein masses that might act as protein aggregation seeds. 1 ml of each of the filtered protein suspensions was continuously shaken at room temperature using an orbital shaker (Superteck, Seolin Science, Korea) rotating at 150 rpm. The shaking-induced aggregation was quantitatively analyzed by determining the turbidity based on the measurements of absorbance at 405 nm every hour. As seen in FIG. 35c, none of hGH-Syn119-140 or the six point mutant hGH-Syn119-140 fusion proteins formed aggregates even after shaking for 40 hours whereas the wild type hGH quickly aggregated within a few hours of shaking.

Finally, stability against repeated freezing/thawing stress was examined in hGH, hGH-Syn119-140 and the point mutant hGH-Syn119-140 fusion proteins. Each protein sample was suspended at a concentration of 1 mg/ml in PBS buffer (pH 7.4). The proteins were induced to aggregate by repeating a cycle of freezing in liquefied nitrogen and thawing in a water bath of 37° C. The extent of the protein aggregation was monitored by measuring absorbance at 405 nm every five freezing/thawing cycles. The wild-type hGH readily aggregates as the number of freezing/thawing cycles increases (FIG. 35d). By contrast, all of hGH-Syn119-140 and the six point mutant Syn119-140-hGH fusion proteins were found to have great stability against repeated freezing/thawing stresses (FIG. 35d).

The results obtained above exhibit that all the point mutants of the ATS peptide have almost the same ability to confer resistance to environmental stresses to fusion partner proteins fused thereto as that of the wild type ATS peptide.

### Example 28

### Resistance of hGH-Synuclein Fusion Proteins (hGH-Synβ113-134 and hGH-Synγ106-127) Aontaining a Fragment of Synβ and Synγ to Stress.

Along with α-synuclein, β- and γ-synuclein, all found in humans, are members of the synuclein family (Lavedan C., Genome Research, 8, 871-880 91998); Lucking C. B and Brice A. Cell Mol Life Sci, 57, 1894-1908 (2000); Iwai A. Biochem. Biophys. Acta, 1502, 95-109 (2000); Hashimoto M. and Masliah E. Brain Pathol. 9, 707-720 (1999)). Whether hGH-Synβ113-134, containing a fragment of Synβ and hGH-Synγ106-127, containing a fragment of Syny, are resistant to stresses or not was examined.

DNA constructs encoding hGH-Synβ113-134 and hGH-Synγ106-127 were prepared by subcloning, instead of a gene encoding the Syn119-140 peptide, genes encoding Synβ113-134(amino acid residues 113-134 of β-synuclein) or Synγ106-127(amino acid residues 106-127 of γ-synuclein). In brief, two DNA fragments respectively encoding Synβ and Synγ were amplified by PCR using a set of two primers having BamHI and EcoRI restriction sites, respectively (Table 13). After being cut with the restriction enzymes BamHI and EcoRI, each of the PCR products was ligated to the hGH gene of DNA construct for hGH-Syn119-140 which had already been digested with the same restriction enzymes, so as to produce DNA constructs coding for hGH-Synβ113-134 and hGH-Synγ106-127 fusion proteins. All DNA constructs were identified for their sequences by DNA sequencing.

TABLE 13

Primers for use in the preparation of expression
vectors for fusion proteins hGH-Synβ113-134 and
hGH-Synγ106-127

| | | |
|---|---|---|
| Genes | Primers | Primer Sequence |

| | | | |
|---|---|---|---|
| hGH-Synβ113-134 | ATSB-BamH1-F | 5′-GGA CTT CC GGA TCC GAG | (SEQ ID NO:66) |
| | | CCA GAA GGG GAG AGT | |
| hGH-Synβ113-134 | ATSB-EcoR1-R | 5′-AAG CTT GAA TTC TCA CGC | (SEQ ID NO:67) |
| | | CTC TGG CTC ATA CTC | |
| hGH-Synγ106-127 | ATSG-BamH1-F | 5′-GGA ATT CC GGA TCC CAA | (SEQ ID NO:68) |
| | | CAG GAG GGT GTG GCA | |
| hGH-Synγ106-127 | ATSG-EcoR1-R | 5′-AAG CTT GAA TTC TCA GTC | (SEQ ID NO:69) |
| | | TCC CCC ACT CTG GGC | |

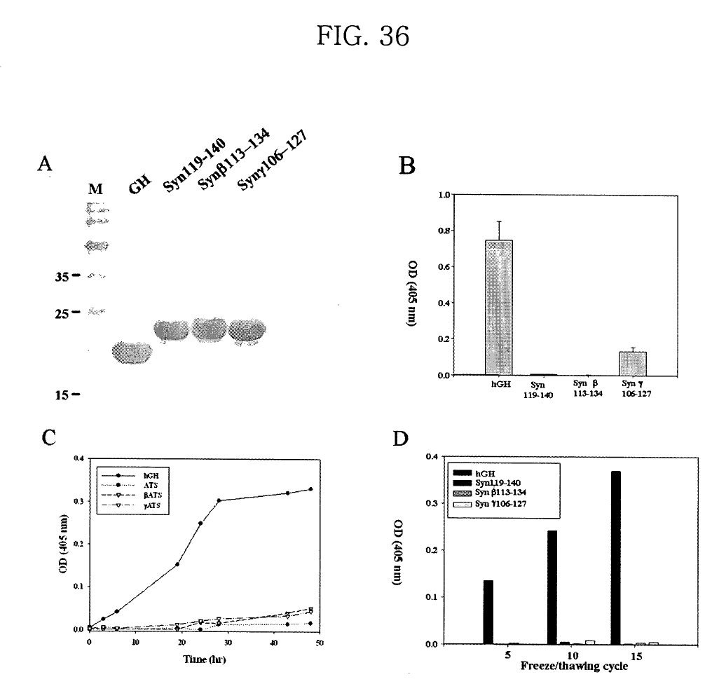

Both hGH-Synβ113-134 (SEQ ID NO:102) and hGH-Synγ106-127 (SEQ ID NO:103) were overexpressed at similar expression levels in E. coli, forming inclusion bodies composed of insoluble aggregates of the expressed proteins. The inclusion bodies were recovered by a somewhat modified version of the Kim et al. method (Kim, J., Chwae, Y. J., Kim, M. Y., Choi, I. H., Park, J. H., Kim, S. J. (1997) J. Immunol. 159, 3875-3882). The refolding of the proteins was achieved by a somewhat modified version of Patra et al's alkali method (Patra, A. K., Mukhopadhyay, R., Mukhija, R., Krishnan, A., Garg, L. C., Panda, A. K. (2000) Prot. Exp. Purif. 18, 182-192), followed by conducting a column chromatography to purify the proteins. Both hGH-Synβ and hGH-Synγ were found to have a size of 24 kDa as analyzed by SDS-PAGE (FIG. 36a). MALDI-TOF mass spectrometry showed that the measured molecular weights of ATS fusion proteins are identical to those calculated from the amino acid sequences (data not shown).

Amino acid sequences of the Syn peptides tested are listed in Table 14, below. Between Synα119-140 and Synβ113-134 peptides, sequence identity and sequence similarity were found to be about 59% and 81%, respectively. To the Synα109-140 peptide, the Synγ106-127 peptide was found to be about 14% in sequence identity and about 36% in sequence similarity.

Table 14

Amino acid sequences of Syn119-140,
Synβ113-134 and Synγ106-127

Sequence

| | | |
|---|---|---|
| Syn119-140 | DPDNEAYEMPSEEGYQDYEPEA | (SEQ ID NO:7) |
| | (aa res. 119-240 of | |
| | α-synuclein) | |
| Synβ113-134 | EPEGESYEDPPQEEYQEYEPEA | (SEQ ID NO:17) |
| | (aa res. 113-134 of | |
| | β-synuclein) | |
| Synγ106-127 | QQEGVASKEKEEVAEEAQSGGD | (SEQ ID NO:18) |
| | (aa res. 106-127 of | |
| | γ-synuclein) | |

The hGH-Synβ113-134 and hGH-Synγ106-127 fusion proteins, along with the fusion protein hGH-Syn119-140 and the wild type hGH, were analyzed for resistance to environmental stresses.

First, the heat-induced aggregation of the proteins was compared. A suspension of each of the protein samples (I mg/ml) in PBS buffer was treated on a hot plate at 100° C. for 10 min, allowed to stand at room temperature for 10 min, and measured for apparent absorbance at 405 nm. As seen in FIG. 36b, hGH completely aggregated whereas all of the hGH-ATS fusion proteins seldom aggregated even after heat treatment at 100° C. for 10 min. The heat resistance of hGH-Synγ106-127 is somewhat poor compared with that of hGH-Syn119-140 or hGH-Synβ113-134, but much higher than that of the wild type hGH.

Next, the shaking-induced aggregation of the hGH-ATSβ113-134 and ATSγ106-127 fusion proteins was examined. A suspension of 1 mg/ml of each of the proteins in PBS buffer (pH 7.4) was allowed to pass through a 0.2 μm syringe filter to remove any protein masses that might act as protein aggregation seeds. 1 ml of each of the filtered protein suspensions was continuously shaken at room temperature using an orbital shaker (Superteck, Seolin Science, Korea) rotating at 150 rpm. The shaking-induced aggregation was quantitatively analyzed by determining the turbidity based on the measurements of absorbance at 405 nm every hour. As seen in FIG. 36c, none of the hGH-ATS fusion proteins formed aggregates even after shaking for 50 hours whereas the wild type hGH quickly aggregated within a few hours of shaking.

Finally, stability against repeated freezing/thawing stress was examined in hGH, hGH-Syn119-140, hGH-ATSβ113-134 and ATSγ106-127 fusion proteins. Each protein sample was prepared at a concentration of 1 mg/ml in PBS buffer (pH 7.4). The proteins were induced to aggregate by repeating a cycle of freezing in liquefied nitrogen and thawing in a water bath at 37° C. The extent of protein aggregation was monitored by measuring absorbance at 405 nm every five freezing/thawing cycles. The wild-type hGH readily aggregates as the number of the freezing/thawing cycles increases (FIG. 36d). By contrast, all of hGH, hGH-Syn119-140, hGH-ATSβ113-134 and ATSγ106-127 fusion proteins were found to have great stability against repeated freezing/thawing stresses (FIG. 36d).

Taken together, the results obtained above exhibit that, like the intact Syn119-140 peptide, both hGH-ATSβ113-134 and ATSγ106-127 have the ability to confer resistance to environmental stresses to fusion partner proteins fused thereto, thereby guaranteeing the activity of the fusion proteins in vivo as well as in vitro. When account is taken of the results, ATS peptides (all of ATSα, ATSβ, ATSγ) derived from the synuclein of other animal origins are expected to have the same functionality as those of human origin. Also, based on the fact that, although ATSβor ATSγ is as low as 14-59% in sequence identity to ATS and as low as 36-81% in sequence similarity to ATS, all of them maintain their resistance to stresses, any synthetic peptide, if similar to ATS, is expected to have similar functionality.

### Example 29

### Stabilization of GCSF by Syn119-140 Peptide Fusion

To examine whether therapeutic proteins other than hGH can be stabilized to environmental stresses by fusion with ATS peptides, a fusion protein GCSF-Syn119-140 (SEQ ID NO: 104) containing Syn119-140 at the C-terminus of GCSF was prepared.

In this regard, a human GCSF gene was cloned from erythrocytes by PCR. For this, 100 ml of blood taken from a healthy adult was diluted 1:1 in an RPMI-1640 medium and then the dilution was carefully layered onto Ficoll-hypaque to induce layer separation. PBMC were separated by gradient centrifugation at 2,000 g for 25 min. From 1×10⁷ PBMC, total RNA was isolated by the guanidine isothiocynate-phenol-chloroform extraction method. cDNA was prepared by reacting 1-5 μg of the total RNA with RNA PCR Kit (AMV) ver2.1 (TaKaRa Bio Inc., Japan) at 42° C. for one hour.

10 μl of the cDNA containing a gene encoding GCSF was amplified by PCR using a set of a 5′ primer having an NdeI recognition site and a 3′ primer having HindIII recognition site, and the PCR product, after being cut with the restriction enzymes NdeI and HindIII, was inserted into pRSETA (Invitrogen) to construct a GCSF expression vector.

A GCSF-Syn119-140 DNA construct was prepared by subcloning an hGCSF gene and, subsequently, a gene encoding the Syn119-140. In brief, a chemically synthesized DNA encoding ATS was inserted into a pRSETA vector (pATS-C) using the restriction sites BamHI and HindIII and a DNA region coding for GCSF was subcloned into pATS-C using the restriction sites NdeI and BamHI. Chemically synthesized DNA sequences of primers given in Table 15, below.

TABLE 15
DNA primer sequences for preparation of GCSF and GCSF-ATS expression vectors

| | | |
|---|---|---|
| Vector | Primers | Sequence |

| | | | |
|---|---|---|---|
| GCSF | GCSF-NdeI | 5′-ACA GTC TCA [symbol] ACC CCC CTA GGA CCT | (SEQ ID NO:70) |
| GCSF | GCSF-HindIII | 5′-GTT TCA [symbol] TCA GGG CTG GGC AAG | (SEQ ID NO:71) |
| GCSF- | GCSF-BamHI-R | 5′-GTT TCA [symbol] GGG CTG GGC AAG GTG | (SEQ ID NO:72) |
| syn119-140 | | | |

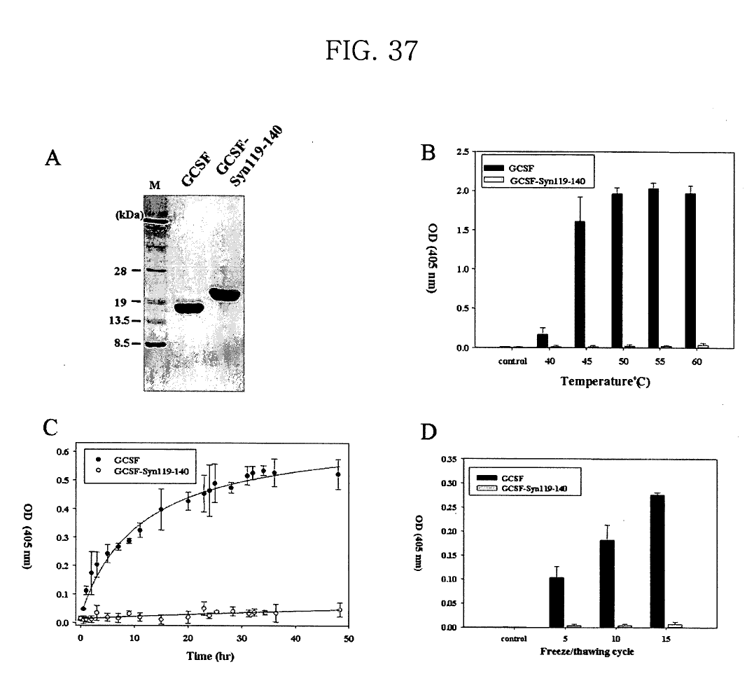

The GCSF expression vector and the GCSF-Syn119-140 expression vector were introduced into E. coli to produce each protein of interest. Culturing the transformed E. coli resulted in the overexpression of the wild type GCSF protein as insoluble aggregates but of the GCSF-Syn119-140 fusion protein as soluble forms. The inclusion body of the expressed wild type GCSF-Syn119-140 was recovered by a somewhat modified version of the Lu et al method (Lu H S, Clogston C L, Narhi L O, Merewether L A, Pearl W R, Boone T C.(1992), JBC. 267, 8770-8777) and then refolded by the copper oxidation method of Souza (Souza, L M. (1989), U.S. Pat. No. 4,810,643), followed by the purification of the refolded proteins by column chromatography. The purified protein was found to have a size of 18.8 kDa as analyzed by SDS-PAGE (FIG. 37a). The recovery of the soluble ATS-fused GCSF was achieved by lysing the E. coli, precipitating with 30% ammonium sulfate, and centrifuging the cell lysate. The GCSF-ATS obtained as a pellet was refolded by a somewhat modified version of the copper oxidation method (Souza, L M. (1989), U.S. Pat. No. 4,810,643), followed by purification through general column chromatography. The purified protein was found to have a size of 21.5 kDa as measured by SDS-PAGE (FIG. 37a). MALDI-TOF mass spectrometry showed that the measured molecular weights of the wild type GCSF and the ATS fusion proteins are identical to those calculated from their respective amino acid sequences (data not shown).

The resistances to environmental stresses of the GCSF and the GCSF-Syn119-140 were compared.

First, the proteins were examined for heat-induced aggregation. A suspension of each of the protein samples (1 mg/ml) in PBS buffer was treated on a hot plate for 10 min, allowed to stand at room temperature for 10 min, and measured for apparent absorbance at 405 nm. As seen in FIG. 37b, the GCSF-Syn119-140 fusion protein did not aggregate at all even after heat treatment in the range of 40° C. to 60° C. for 10 min whereas the aggregation of GCSF started at 40° C. and was completed at 45° C. In addition, even heat treatment at 100° C. for 10 min did not aggregate the GCSF-Syn119-140 fusion protein at all (data not shown).

Next, how the wild type GCSF and the GCSF-Syn119-140 fusion protein are induced to aggregate upon shaking was examined. A suspension of 1 mg/ml of each of the proteins in PBS buffer (pH 7.4) was passed through a 0.2 μm syringe filter to remove any protein masses that might act as protein aggregation seeds. 1 ml of each of the filtered protein suspensions was continuously shaken at room temperature using an orbital shaker (Superteck, Seolin Science, Korea) rotating at 150 rpm. The shaking-induced aggregation was quantitatively analyzed by determining the turbidity based on the measurements of absorbance at 405 nm every hour. As seen in FIG. 37c, the GCSF-Syn119-140 fusion protein formed no particular aggregates even after shaking for 50 hours whereas the wild type GCSF quickly aggregated within a few hours of shaking.

Finally, stability against repeated freezing/thawing stress was examined in GCSF and GCSF-Syn119-140. Each protein sample was prepared at a concentration of 1 mg/ml in PBS buffer (pH 7.4). The proteins were induced to aggregate by repeating a cycle of freezing in liquefied nitrogen and thawing in a water bath at 37° C. The extent of protein aggregation was monitored by measuring absorbance at 405 nm every five freezing/thawing cycles. The wild-type GCSF readily aggregates as the number of freezing/thawing cycles increases (FIG. 37d). In contrast, the GCSF-ATS fusion protein did not aggregate as a result of repeated freezing/thawing stresses (FIG. 37d).

These results exhibit that the ATS peptide can be very useful for stabilizing GCSF as well as hGH.

### Example 30

### Stabilization of Human Leptin by Fusion with Syn119-140 Peptide

To verify the ability of the ATS peptide to confer environmental stress resistance to the protein, preferably, a therapeutic protein, which is fused thereto, Syn119-140 was fused to the C-terminus of human leptin to construct an hLeptin-Syn119-140 (SEQ ID NO: 105) fusion protein.

By reverse transcriptase PCR, human leptin cDNA was obtained from the RNA extracted from the adipose tissue. Using a set of a 5′ primer having an NdeI recognition site and a 3′ primer having an EcoRI recognition site (Table 16), PCR was conducted with 10 μl of the cDNA serving as a template. The PCR product was digested with the restriction enzymes NdeI and EcoRI, followed by the insertion of the digested DNA into pRSETA (Invitrogen) to form an expression vector.

An hLeptin-ATS construct was prepared by consecutively subcloning a gene coding for hLeptin and Syn119-140. In brief, for the purpose of C-terminal fusion, a chemically synthesized DNA encoding Syn119-140 was inserted into a pRSETA vector (pATS-C), with the aid of BamHI and HindIII restriction sites. The protein coding region of hLeptin was subcloned into the pATS-C vector with the aid of NdeI and BamHI restriction sites. The chemically synthesized DNA sequences of the primers used for constructing expression vectors for hLeptin and hLeptin-ATS are listed in Table 16, below.

TABLE 16

DNA primer sequences for the preparation of hLeptin and hLeptin-Syn119-140
expression vectors

| | | |
|---|---|---|
| Vectors | Primers | Sequence |

| | | | |
|---|---|---|---|
| hLeptin | hLeptin- | 5′-ACA GTC TCA [symbol] GTG CCC ATC CAA AAA GT | (SEQ IN NO:73) |
| | Nde1 | | |
| hLeptin | hLeptin- | 5′-GTC AAG CTT [symbol] TCA GCA CCC AGG GC | (SEQ IN NO:74) |
| | EcoR1 | | |
| hLeptin-ATS | hLeptin- | 5′-ACA GTC [symbol] GCA CCC AGG GCT GAG | (SEQ IN NO:75) |
| | BamH1-R | | |

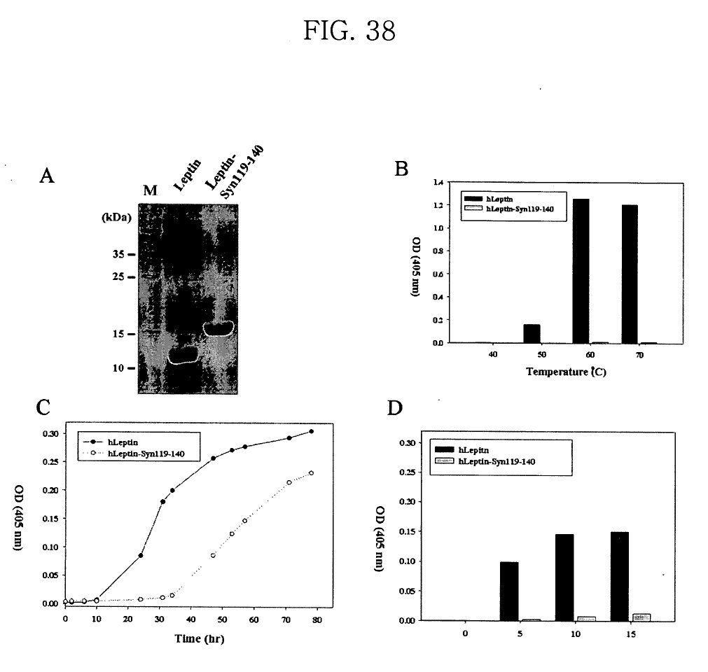

Both hLeptin and hLeptin-ATS were overexpressed at similar expression levels in E. coli, forming inclusion bodies composed of insoluble aggregates of the expressed proteins. The inclusion bodies were recovered by a somewhat modified version of the Kim et al. method (Kim, J., Chwae, Y. J., Kim, M. Y., Choi, I. H., Park, J. H., Kim, S. J. (1997) J. Immunol. 159, 3875-3882), then refolded by a somewhat modified version of Jeong et al's dialysis method (Jeong K J, Lee S Y. (1999) Appl Environ Microbiol. 65, 3027-32.), and finally purified through general column chromatography. On SDS-PAGE, the purified hLeptin was detected at a size of 16 kDa while the purified hLeptin-Syn119-140 was detected at a size of 18 kDa (FIG. 38a). MALDI-TOF mass spectrometry showed that the measured molecular weights of hLeptin and hLeptin-Syn119-140 are identical to those calculated from their respective amino acid sequences (data not shown).

The hLeptin and the hLeptin-Syn119-140 fusion protein were analyzed for resistance to environmental stresses.

First, the proteins were examined for heat-induced aggregation. A suspension of each of the protein samples (1 mg/ml) in PBS buffer was treated on a hot plate for 10 min and allowed to stand at room temperature for 10 min, followed by measuring apparent absorbance at 405 nm to quantitatively determine the heat-induced aggregation levels of the proteins. As seen in FIG. 38b, the hLeptin-Syn119-140 fusion protein did not aggregate at all even after heat treatment in the range of from 40° C. to 70° C. for 10 min whereas hLeptin started to aggregate at 50° C. and completely aggregated at 60° C. In addition, even heat treatment at 100° C. for 10 min did not aggregate the GCSF-Syn1190-140 fusion protein at all (data not shown).

Next, investigation was made into the difference in shaking-induced aggregation between the wild type hLeptin and the fusion protein hLeptin-Syn119-140. A suspension of 1 mg/ml of each of the proteins in PBS buffer (pH 7.4) was passed through a 0.2 μm syringe filter to remove any protein masses that might act as protein aggregation seeds. 1 ml of each of the filtered protein suspensions was continuously shaken at room temperature using an orbital shaker (Superteck, Seolin Science, Korea) rotating at 150 rpm. The shaking-induced aggregation was quantitatively analyzed by determining the turbidity based on the measurements of absorbance at 405 nm every hour. As seen in FIG. 38c, the hLeptin-ATS fusion protein formed no particular aggregates after shaking for 40 hours and then started to aggregate whereas the wild type GCSF quickly aggregated after 10 hours of shaking.

Finally, stability against repeated freezing/thawing stress was examined in hLeptin and hLeptin-ATS. Each protein sample was suspended at a concentration of 1 mg/ml in PBS buffer (pH 7.4). The proteins were induced to aggregate by repeating a cycle of freezing in liquefied nitrogen and thawing in a water bath at 37° C. The extent of the protein aggregation was monitored by measuring absorbance at 405 nm every five freezing/thawing cycles. The wild-type hLeptin readily aggregates with an increase in the number of freezing/thawing cycles (FIG. 38d). By contrast, the hLeptin-Syn119-140 fusion protein did not form any aggregates as a result of repeated freezing/thawing stresses (FIG. 38d).

These results exhibit that the ATS peptide can be very useful in stabilizing GCSF as well as hGH and GSCF. Furthermore, the ATS peptide is believed to be generally useful in stabilizing other therapeutic proteins, as well.

### Example 31

### Effect of fusion with ATS Peptide on Protein Solubility

One quite distinct feature of the ATS amino acid sequence is redundancy in negatively charged amino acid residues such as Glu or Asp so that ATS has low pI values. As a rule, because the solubility of a protein is proportional to the square of its net charges (Tanford, 1961, in Physical Chemistry of macromolecules), an ATS fusion protein is expected to increase in solubility compared to the wild type.

To verify this expectation, various proteins were examined for solubility difference according to the presence or absence of the Syn119-140 peptide. GST, hGH and hLeptin, and fusion proteins containing Syn119-140 fused to the C-termini thereof, all having 95% or higher purity, were quantitatively analyzed by measuring their solubilities. The same volumes of the solutions of the proteins were centrifuged at 9,000 g for 10 min in a Centricon centrifugal device (Amicon, Beverly, Mass.), and the supernatants were quantitatively measured for protein concentration by the Bradford method. Using the remaining samples, the same test was further conducted 5-7 times until the centrifugation yielded no more protein, followed by quantitative measurement by the Bradford method.

It is apparent from the results shown in Table 17 that Syn119-140 fusion proteins have far higher solubility than do the wild type proteins. Fusion with an Syn119-140 peptide increased solubility by 20% for GST, about two fold for hGH, and about five fold for hLeptin. Particularly, both hGH and hLeptin are precipitated at high concentrations, but no precipitation was observed in the Syn119-140 fusion protein. Generally, when fused to the Syn119-140 peptide, proteins having low solubility (hLeptin or hGH) tend to increase their solubility to a larger extent compared to proteins having high solubility (GST)

TABLE 17
Solubility difference according to fusion with Syn119-140 peptide

| protein | solubility | | precipitation |
|---|---|---|---|
| GST | [symbol] 200 | mg/ml | No |
| GST-Syn119-140 | [symbol] 250 | mg/ml | No |
| hGH | [symbol] 80 | mg/ml | Yes |
| hGH-Syn119-140 | [symbol] 150 | mg/ml | No |
| hLeptin | [symbol] 20 | mg/ml | Yes |
| hLeptin-Syn119-140 | [symbol] 100 | mg/ml | No |

Taken together, the results demonstrate that ATS-derived peptides are useful in increasing the solubility as well as stability of the proteins of interest. Usually administered to patients through injection, therapeutic proteins need to be formulated in injection dosage forms that are highly concentrated so as to be administered in low amounts for patients' convenience. The improvement in the solubility of therapeutic protein medicines by fusion with the ATS peptides satisfies the necessity.

As described hereinbefore, the peptide fragment which contains ten consecutive amino acid residues having a sequence composed of 10 or more consecutive amino acid residues, including five or more acidic amino acid residues derived from the C-terminal acidic tail of synuclein (ATS), or its derivatives according to the present invention can not only confer resistance to environmental stresses to a protein that is fused thereto, without deteriorating intrinsic properties of the fused protein, but also increase the solubility of the protein. Therefore, when fused to the peptides of the present invention, a protein of interest can have a prolonged half life and be effectively used in vivo as well as in vitro, without the loss of its functionality.

### INDUSTRIAL APPLICABILITY

With the advantage of improved environmental stress resistance and increased solubility, the peptide which contains ten consecutive amino acid residues having a sequence composed of 10 or more consecutive amino acid residues, including five or more acidic amino acid residues derived from the C-terminal acidic tail of synuclein (ATS), or its derivatives will find useful applications in various fields including medical science, life engineering, food, etc.

1. A peptide conferring resistance to environmental stress, comprising:
(i) a peptide fragment containing a sequence composed of 10 or more consecutive amino acid residues including five or more acidic amino acid residues, wherein the peptide fragment is derived from SEQ ID NO:1 corresponding to amino acid residues 96-140 of the C-terminal acidic tail of α-synuclein, or its derivative,
(ii) a peptide fragment containing a sequence composed of 10 or more consecutive amino acid residues including five or more acidic amino acid residues, wherein the peptide fragment is derived from SEQ ID NO:2 corresponding to amino acid residues 85-134 of the C-terminal acidic tail of β-synuclein, or its derivative,
(iii) a peptide fragment containing a sequence composed of 10 or more consecutive amino acid residues including five or more acidic amino acid residues, wherein the peptide fragment is derived from SEQ ID NO:3 corresponding to amino acid residues 96-127 of the C-terminal acidic tail of γ-synuclein, or its derivative, or
(iv) a peptide fragment containing a sequence composed of 10 or more consecutive amino acid residues including five or more acidic amino acid residues, wherein the peptide fragment is derived from SEQ ID NO:4 corresponding to amino acid residues 96-127 of the C-terminal acidic tail of synoretin, or its derivative.

2. The peptide as set forth in claim 1, wherein the peptide fragment derivative of the C-terminal acidic tail of α-synuclein is selected from the group consisting of the mutants of which one or more amino acid residues at residue numbers 122, 123, 124, 127, 133 and 140 are substituted with another amino acid that differs from the original amino acid residue of the C-terminal acidic tail of α-synuclein.

3. The peptide as set forth in claim 1, wherein the peptide fragment corresponding to the C-terminal acidic amino tail of α-synuclein or its derivative is SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO: 15 or SEQ ID NO:16.

4. The peptide as set forth in claim 1, wherein the peptide fragment corresponding to the C-terminal acidic amino tail of β-synuclein or its derivative is SEQ ID NO:17.

5. The peptide as set forth in claim 1, wherein the peptide fragment corresponding to the C-terminal acidic amino tail of γ-synuclein or its derivative is SEQ ID NO:18.

6. The peptide as set forth in claim 1, wherein the environmental stress is selected from the group consisting of heat, pH, metal ions, repeated freezing/thawing, shaking, high concentration of polypeptide, and combinations thereof.

7. A fusion protein, comprising the peptide of claim 1 and a fusion partner protein.

8. The fusion protein as set forth in claim 7, wherein the peptide is linked to the fusion partner protein at such a position as not to affect the intrinsic properties of the fusion partner protein.

9. The fusion protein as set forth in claim 7, wherein the fusion partner protein is labile to an environmental stress.

10. The fusion protein as set forth in claim 7, characterized in that the fusion partner protein in the fusion protein shows decreased denaturation compared to in its natural state.

11. The fusion protein as set forth in claim 6, characterized in that the fusion partner protein in the fusion protein shows increased solubility compared to in its natural state.

12. The fusion protein as set forth in claim 7, wherein the peptide is linked to the N-terminus, the C-terminus or both termini of the fusion partner protein.

13. The fusion protein as set forth in claim 12, wherein the fusion partner protein is selected from the group consisting of hormones, cytokines, enzymes, antibodies, growth factors, transcription factors, blood factors, vaccines, structural proteins, ligand proteins, and receptors.

14. The fusion protein as set forth in claim 13, wherein the fusion partner protein is selected from the group consisting of glutathione S-transferase, dihydrofolate reductase, growth hormones, leptin, growth hormone-releasing peptides, interferons, interferon receptors, colony-stimulating factors, glucagon-like peptides (GLP-1, etc.), G-protein-coupled receptor, interleukins, interleukin receptors, interleukin-associated proteins, cytokine-associated proteins, macrophage-activating factors, macrophage peptides, B-cell factors, T-cell factors, protein A, suppressive factor of allergy, cell necrosis glycoprotein, immune toxins, lymphotoxins, tumor necrosis factors, tumor inhibitory factor, transforming growth factor, alpha-1 antitrypsin, albumin, alpha-lactalbimin, apolipoprotein-E, erythroprotein, hyper-glycosylated erythroprotein, angiopoietins, hemoglobin, thrombin, thrombin receptor activating peptide, thrombomodulin , factor VII , factor VII a, factor VIII, factor IX , factor XIII, plasminogen activator, fibrin binding protein, urokinase, steptokinase, hirudin, protein C, C-reactive protein, renin inhibitor, collagenase inhibitor, superoxide dismutase, leptin, platelet derived growth hormone, epithelial growth factor, epidermal growth factor, angiostatin, angiotensin, osteogenic growth factor, osteogenesis stimulating protein, calcitonin, insulin, atriopeptin, cartilage inducing factor, elcatonin, connective tissue activator protein, tissue factor pathway inhibitor, follicle stimulating hormone, luteinizing hormone, luteinizing hormone-releasing hormone, nerve growth factor, parathyroid hormone, relaxin, secretin, somatomedin, insulin-like growth factor, adrenocorticotrophic hormone, glucagon, cholecystokinin, pancreatic polypeptide, gastrin releasing peptide, corticotropin releasing factor, thyroid stimulating hormone, autotaxin, lactoferrin, myostatin, receptors, receptor antagonists, cell surface antigens, virus-derived vaccine antigens, monoclonal antibodies, polyclonal antibodies, and antibody fragments.

15. A method of conferring resistance to environmental stress to a protein of interest, comprising linking the protein to the peptide of claim 1.


SEQUENCE LISTING (105 sequences)

SEQ ID NO: 1 - protein, 45 aa, homo sapiens
  PEPTIDE (1)..(45): Syn(alpha)96-140
KKDQLGKNEEGAPQEGILEDMPVDPDNEAYEMPSEEGYQDYEPEA

SEQ ID NO: 2 - protein, 50 aa, homo sapiens
  PEPTIDE (1)..(50): Syn(beta)85-134
KREEFPTDLKPEEVAQEAAEEPLIEPLMEPEGESYEDPPQEEYQEYEPEA

SEQ ID NO: 3 - protein, 32 aa, homo sapiens
  PEPTIDE (1)..(32): Syn(gamma)96-127
RKEDLRPSAPQQEGVASKEKEEVAEEAQSGGD

SEQ ID NO: 4 - protein, 32 aa, Homo sapiens
  PEPTIDE (1)..(32): Synoretin96-127
HKEALKQPVPPQEDEAAKAEEQVAEETKSGGD

SEQ ID NO: 5 - protein, 13 aa, homo sapiens
  PEPTIDE (1)..(13): Syn(alpha)103-115
NEEGAPQEGILED

SEQ ID NO: 6 - protein, 13 aa, homo sapiens
  PEPTIDE (1)..(13): Syn(alpha)114-126
EDMPVDPDNEAYE

SEQ ID NO: 7 - protein, 22 aa, homo sapiens
  PEPTIDE (1)..(22): Syn(alpha)119-140
DPDNEAYEMPSEEGYQDYEPEA

SEQ ID NO: 8 - protein, 11 aa, homo sapiens
  PEPTIDE (1)..(11): Syn(alpha)130-140
EEGYQDYEPEA

SEQ ID NO: 9 - protein, 28 aa, homo sapiens
  PEPTIDE (1)..(28): Syn(alpha)113-140
LEDMPVDPDNEAYEMPSEEGYQDYEPEA

SEQ ID NO: 10 - protein, 17 aa, homo sapiens
  PEPTIDE (1)..(17): Syn(alpha)119-135
DPDNEAYEMPSEEGYQD

SEQ ID NO: 11 - protein, 22 aa, homo sapiens
  PEPTIDE (1)..(22): E123A mutant of Syn(alpha)119-140
DPDNAAYEMPSEEGYQDYEPEA

SEQ ID NO: 12 - protein, 22 aa, homo sapiens
  PEPTIDE (1)..(22): Y133A mutant of Syn(alpha)119-140
DPDNEAYEMPSEEGAQDYEPEA

SEQ ID NO: 13 - protein, 22 aa, homo sapiens
  PEPTIDE (1)..(22): A124E mutant of Syn(alpha)119-140
DPDNEEYEMPSEEGYQDYEPEA

SEQ ID NO: 14 - protein, 22 aa, homo sapiens
  PEPTIDE (1)..(22): N122V mutant of Syn(alpha)119-140
DPDVEAYEMPSEEGYQDYEPEA

SEQ ID NO: 15 - protein, 22 aa, homo sapiens
  PEPTIDE (1)..(22): M127S mutant of Syn(alpha)119-140
DPDNEAYESPSEEGYQDYEPEA

SEQ ID NO: 16 - protein, 22 aa, homo sapiens
  PEPTIDE (1)..(22): A140S mutant of Syn(alpha)119-140
DPDNEAYEMPSEEGYQDYEPES

SEQ ID NO: 17 - protein, 22 aa, homo sapiens
  PEPTIDE (1)..(22): Syn(beta)113-134
EPEGESYEDPPQEEYQEYEPEA

SEQ ID NO: 18 - protein, 22 aa, homo sapiens
  PEPTIDE (1)..(22): Syn(gamma)106-127
QQEGVASKEKEEVAEEAQSGGD

SEQ ID NO: 19 - DNA, 36 bp, Artificial Sequence
  primer for constructing GST-Synuclein fusion protein
gcgctcgagccagatctgccatggatgtattcatga

SEQ ID NO: 20 - DNA, 36 bp, Artificial Sequence
  primer for constructing GST-Synuclein fusion protein
gcgcaagcttgtcgacttaggcttcaggttcgtagt

SEQ ID NO: 21 - DNA, 37 bp, Artificial Sequence
  Primer for constructing GST-Synuclein fusion protein
gcgcaagcttgtcgacctatttggtcttctcagccac

SEQ ID NO: 22 - DNA, 28 bp, Artificial Sequence
  Primer for constructing GST-Synuclein fusion protein
gcgcagatctcatatggagcaagtgaca

SEQ ID NO: 23 - DNA, 34 bp, Artificial Sequence
  Primer for constructing GST-Synuclein fusion protein
gcgcaagcttgtcgacctagacttagccagtggc

SEQ ID NO: 24 - DNA, 40 bp, Artificial Sequence
  Primer for constructing GST-Synuclein fusion protein
gcgcggtaccgagatctggatgaaaaaggaccagttgggc

SEQ ID NO: 25 - DNA, 36 bp, Artificial Sequence; identical to SEQ ID NO: 20
  Primer for constructing GST-Synuclein fusion protein

SEQ ID NO: 26 - DNA, 32 bp, Artificial Sequence
  Primer for constructing DHFR-Synuclein fusion protein
gcgcggtaccaaggaccagttgggcaagaatg

SEQ ID NO: 27 - DNA, 30 bp, Artificial Sequence
  Primer for constructing DHFR-Synuclein fusion protein
gcgcgtcgacttaggcttcaggttcgtagt

SEQ ID NO: 28 - DNA, 48 bp, Artificial Sequence
  Primer for constructing peptide fragment of GST-ATS(alpha)103-115 fusion protein
gatccaatgaagaaggagccccacaggaaggcattctggaagattaag

SEQ ID NO: 29 - DNA, 48 bp, Artificial Sequence
  Primer for constructing peptide fragment GST-ATS(alpha)103-115 fusion protein
aattcttaatcttccagaatgccttcctgtggggctccttcttcattg

SEQ ID NO: 30 - DNA, 48 bp, Artificial Sequence
  Primer for constructing peptide fragment of GST-ATS(alpha)114-126 fusion protein
gatccgaagatatgcctgtagatcctgacaatgaggcttatgaataag

SEQ ID NO: 31 - DNA, 48 bp, Artificial Sequence
  Primer for constructing peptide fragment of GST-ATS(alpha)114-126 fusion protein
aattcttattcataagcctcattgtcaggatctacaggcatatcttcg

SEQ ID NO: 32 - DNA, 75 bp, Artificial Sequence
  Primer for constructing peptide fragment of GST-ATS(alpha)119-140 fusion protein
gatccgatcctgacaatgaggcttatgaaatgccttctgaggaagggtatcaagactacgaacctgaagcctaag

SEQ ID NO: 33 - DNA, 75 bp, Artificial Sequence
  Primer for constructing peptide fragment of GST-ATS(alpha)119-140 fusion protein
aattcttaggcttcaggttcgtagtcttgatacccttcctcagaaggcatttcataagcctcattgtcaggatcg

SEQ ID NO: 34 - DNA, 42 bp, Artificial Sequence
  Primer for constructing peptide fragment of GST-ATS(alpha)130-140 fusion protein
gatccgaggaagggtatcaagactacgaacctgaagcctaag

SEQ ID NO: 35 - DNA, 42 bp, Artificial Sequence
  Primer for constructing peptide fragment of GST-ATS(alpha)130-140 fusion protein
aattcttaggcttcaggttcgtagtcttgatacccttcctcg

SEQ ID NO: 36 - DNA, 28 bp, Artificial Sequence
  Primer for constructing GST-ATS(beta) fusion protein
agctaaggatccaagagggaggaattcc

SEQ ID NO: 37 - DNA, 30 bp, Artificial Sequence
  Primer for constructing GST-ATS(beta) fusion protein
aagtaactcgagctacgcctctggctcata

SEQ ID NO: 38 - DNA, 28 bp, Artificial Sequence
  Primer for constructing GST-ATS(gamma) fusion protein
aagaatggatcccgcaaggaggacttga

SEQ ID NO: 39 - DNA, 29 bp, Artificial Sequence
  Primer for constructing GST-ATS(gamma) fusion protein
aatagcgaattcctagtctcccccactct

SEQ ID NO: 40 - DNA, 23 bp, Artificial Sequence
  Primer for constructing GST-E5 fusion protein
gatccgaagaagaagaagaataa

SEQ ID NO: 41 - DNA, 24 bp, Artificial Sequence
  Primer for constructing GST-E5 fusion protein
aattcttattcttcttcttcttcg

SEQ ID NO: 42 - DNA, 39 bp, Artificial Sequence
  Primer for constructing GST-E10 fusion protein
gatccgaagaagaagaagaagaagaagaagaagaataag

SEQ ID NO: 43 - DNA, 39 bp, Artificial Sequence
  Primer for constructing GST-E10 fusion protein
aattcttattcttcttcttcttcttcttcttcttcttcg

SEQ ID NO: 44 - DNA, 32 bp, Artificial Sequence
  Primer for amplification of hGH
gcgctcgagcccatatgttcccaactatacca

SEQ ID NO: 45 - DNA, 34 bp, Artificial Sequence
  Primer for amplification of hGH
gcgcaagcttaagcttttagaagccacagctgcc

SEQ ID NO: 46 - DNA, 71 bp, Artificial Sequence
  Sequence for constructing Syn119-140-hGH fusion protein
tatggatcctgacaatgaggcttatgaaatgccttctgaggaagggtatcaagactacgaacctgaagccg

SEQ ID NO: 47 - DNA, 73 bp, Artificial Sequence
  Sequence for constructing Syn119-140-hGH fusion protein
acctaggactgttactccgaatactttacggaagactccttcccatagttctgatgcttggacttcggcctag

SEQ ID NO: 48 - DNA, 75 bp, Artificial Sequence
  Sequence for constructing hGH-Syn119-140
gatccgatcctgacaatgaggcttatgaaatgccttctgaggaagggtatcaagactacgaacctgaagcctaaa

SEQ ID NO: 49 - DNA, 75 bp, Artificial Sequence
  Sequence for constructing hGH-Syn119-140
gctaggactgttactccgaatactttacggaagactccttcccatagttctgatgcttggacttcggattttcga

SEQ ID NO: 50 - DNA, 27 bp, Artificial Sequence
  ATSw-BamH 1 primer
gcaactggatccgaagatatgcctgtg

SEQ ID NO: 51 - DNA, 27 bp, Artificial Sequence
  ATSw-EcoR 1 primer
actgccgaattcttaggcttcaggttc

SEQ ID NO: 52 - DNA, 27 bp, Artificial Sequence
  ATSp-BamH 1 primer
gcaactggatccgatcctgacaatgag

SEQ ID NO: 53 - DNA, 27 bp, Artificial Sequence
  ATSp-EcoR 1 primer
actgccgaattcttagtcttgataccc

SEQ ID NO: 54 - DNA, 24 bp, Artificial Sequence
  Primer for site-directed mutagenesis to E123A
cctgacaatgcggcttatgaaatg

SEQ ID NO: 55 - DNA, 24 bp, Artificial Sequence
  Primer for site-directed mutagenesis to E123A
catttcataagccgcattgtcagg

SEQ ID NO: 56 - DNA, 21 bp, Artificial Sequence
  Primer for site-directed mutagenesis to Y1334
gaggaaggggctcaagactac

SEQ ID NO: 57 - DNA, 21 bp, Artificial Sequence
  Primer for site-directed mutagenesis to Y1334
gtagtcttgagccccttcctc

SEQ ID NO: 58 - DNA, 21 bp, Artificial Sequence
  Primer for site-directed mutagenesis to Y124E
gacaatgaggaatatgaaatg

SEQ ID NO: 59 - DNA, 21 bp, Artificial Sequence
  Primer for site-directed mutagenesis to Y124E
catttcatattcctcattgtc

SEQ ID NO: 60 - DNA, 22 bp, Artificial Sequence
  Primer for site-directed mutagenesis to N122V
gatcctgacgtggaggcttatg

SEQ ID NO: 61 - DNA, 22 bp, Artificial Sequence
  Primer for site-directed mutagenesis to N122V
cataagcctccacgtcaggatc

SEQ ID NO: 62 - DNA, 21 bp, Artificial Sequence
  Primer for site-directed mutagenesis to M127S
gcttatgaaagcccttctgag

SEQ ID NO: 63 - DNA, 21 bp, Artificial Sequence
  Primer for site-directed mutagenesis to M127S
ctcagaagggctttcataagc

SEQ ID NO: 64 - DNA, 22 bp, Artificial Sequence
  Primer for site-directed mutagenesis to A140S
gaacctgaaagcggatccttcc

SEQ ID NO: 65 - DNA, 22 bp, Artificial Sequence
  Primer for site-directed mutagenesis to A140S
ggaaggatccgctttcaggttc

SEQ ID NO: 66 - DNA, 32 bp, Artificial Sequence
  ATSB-BamH1-F
ggacttccggatccgagccagaaggggagagt

SEQ ID NO: 67 - DNA, 33 bp, Artificial Sequence
  ATSB-EcoR1-R
aagcttgaattctcacgcctctggctcatactc

SEQ ID NO: 68 - DNA, 32 bp, Artificial Sequence
  ATSG-BamH1-F
ggaattccggatcccaacaggagggtgtggca

SEQ ID NO: 69 - DNA, 33 bp, Artificial Sequence
  ATSG-EcoR1-R
aagcttgaattctcagtctcccccactctgggc

SEQ ID NO: 70 - DNA, 30 bp, Artificial Sequence
  GCSF-Nde1 primer
acagtctcacatatgacccccctaggacct

SEQ ID NO: 71 - DNA, 27 bp, Artificial Sequence
  GCSF-Hind 3 primer
gtttcaaagctttcagggctgggcaag

SEQ ID NO: 72 - DNA, 27 bp, Artificial Sequence
  GCSF-BamH1-R primer
gtttcaggatccgggctgggcaaggtg

SEQ ID NO: 73 - DNA, 32 bp, Artificial Sequence
  hLeptin-Nde1 primer
acagtctcacatatggtgcccatccaaaaagt

SEQ ID NO: 74 - DNA, 29 bp, Artificial Sequence
  hLeptin-EcoR1 primer
gtcaagcttgaattctcagcacccagggc

SEQ ID NO: 75 - DNA, 27 bp, Artificial Sequence
  hLeptin-BamH1-R primer
acagtcggatccgcacccagggctgag

SEQ ID NO: 76 - protein, 366 aa, Artificial Sequence
  GST-Syn1-140 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSMDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 77 - protein, 286 aa, Artificial Sequence
  GST-Syn1-60 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSMDVFMKGLSKAKEGVVAAAEKTKQGVAEAAGKTKEGVLYVGSKTKEGVVHGVATVAEKTK

SEQ ID NO: 78 - protein, 261 aa, Artificial Sequence
  GST-Syn61-95 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSEQVTNVGGAVVTGVTAVAQKTVEGAGSIAAATGFV

SEQ ID NO: 79 - protein, 306 aa, Artificial Sequence
  GST-Syn61-140 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSEQVTNVGGAVVTGVTAVAQKTVEGAGSIAAATGFVKKDQLGKNEEGAPQEGILEDMPVDPDNEAYEMPSEEGYQDYEPEA

SEQ ID NO: 80 - protein, 271 aa, Artificial Sequence
  GST-Syn96-140 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSKKDQLGKNEEGAPQEGILEDMPVDPDNEAYEMPSEEGYQDYEPEA

SEQ ID NO: 81 - protein, 234 aa, Artificial Sequence
  DHFR-Syn96-140 fusion protein
MVRPLNCIVAVSQDMGIGKNGDLPWPPLRNEWKYFQRMTTTSSVEGKQNLVIMGRKTWFSIPEKNRPLKDRINIVLSRELKEPPRGAHFLAKSLDDALRLIEQPELASKVDMVWIVGGSSVYQEAMNQPGHLRLFVTRIMQEFESDTFFPEIDLGKYKLLPEYPGVLSEVQEEKGIKYKFEVYEKKDGSKKDQLGKNEEGAPQEGILEDMPVDPDNEAYEMPSEEGYQDYEPEA

SEQ ID NO: 82 - protein, 239 aa, Artificial Sequence
  GST-Syn103-115 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSNEEGAPQEGILED

SEQ ID NO: 83 - protein, 239 aa, Artificial Sequence
  GST-Syn114-126 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSEDMPVDPDNEAYE

SEQ ID NO: 84 - protein, 248 aa, Artificial Sequence
  GST-Syn119-140 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSDPDNEAYEMPSEEGYQDYEPEA

SEQ ID NO: 85 - protein, 237 aa, Artificial Sequence
  GST-Syn130-140 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSEEGYQDYEPEA

SEQ ID NO: 86 - protein, 276 aa, Artificial Sequence
  GST-Syn(beta) fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSKREEFPTDLKPEEVAQEAAEEPLIEPLMEPEGESYEDPPQEEYQEYEPEA

SEQ ID NO: 87 - protein, 258 aa, Artificial Sequence
  GST-Syn(gamma) fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSRKEDLRPSAPQQEGVASKEKEEVAEEAQSGGD

SEQ ID NO: 88 - protein, 231 aa, Artificial Sequence
  GST- E5 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSEEEEE

SEQ ID NO: 89 - protein, 236 aa, Artificial Sequence
  GST- E10 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSEEEEEEEEEE

SEQ ID NO: 90 - DNA, 648 bp, Artificial Sequence
  Synthetic Construct
atgttcccaactataccactatctcgtctattcgataacgctatgcttcgtgctcatcgtcttcatcagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcggatccgatcctgacaatgaggcttatgaaatgccttctgaggaagggtatcaagactacgaacctgaagcc

SEQ ID NO: 91 - protein, 216 aa, Artificial Sequence
  Synthetic Construct for hGH-Syn119-140 fusion protein
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGSDPDNEAYEMPSEEGYQDYEPEA

SEQ ID NO: 92 - DNA, 648 bp, Artificial Sequence
  Synthetic Construct
atggatcctgacaatgaggcttatgaaatgccttctgaggaagggtatcaagactacgaacctgaagccggatccttcccaactataccactatctcgtctattcgataacgctatgcttcgtgctcatcgtcttcatcagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttc

SEQ ID NO: 93 - protein, 216 aa, Artificial Sequence
  Synthetic Construct for Syn119-140-hGH fusion protein
MDPDNEAYEMPSEEGYQDYEPEAGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 94 - protein, 222 aa, Artificial Sequence
  hGH-Syn113-140 fusion protein
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGSLEDMPVDPDNEAYEMPSEEGYQDYEPEA

SEQ ID NO: 95 - protein, 213 aa, Artificial Sequence
  hGH-Syn119-135 fusion protein
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGSDPDNEAYEMPSEEGYQDYE

SEQ ID NO: 96 - protein, 216 aa, Artificial Sequence
  hGH-SynE123A fusion protein
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGSDPDNAAYEMPSEEGYQDYEPEA

SEQ ID NO: 97 - protein, 216 aa, Artificial Sequence
  hGH-SynY133A fusion protein
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGSDPDNEAYEMPSEEGAQDYEPEA

SEQ ID NO: 98 - protein, 216 aa, Artificial Sequence
  hGH-SynA124E fusion protein
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGSDPDNEEYEMPSEEGYQDYEPEA

SEQ ID NO: 99 - protein, 216 aa, Artificial Sequence
  hGH-Syn122V fusion protein
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGSDPDVEAYEMPSEEGYQDYEPEA

SEQ ID NO: 100 - protein, 216 aa, Artificial Sequence
  hGH-SynM127S fusion protein
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGSDPDNEAYESPSEEGYQDYEPEA

SEQ ID NO: 101 - protein, 216 aa, Artificial Sequence
  hGH-SynA140S fusion protein
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGSDPDNEAYEMPSEEGYQDYEPES

SEQ ID NO: 102 - protein, 216 aa, Artificial Sequence
  hGH-Syn(beta) 113-134 fusion protein
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGSEPEGESYEDPPQEEYQEYEPEA

SEQ ID NO: 103 - protein, 216 aa, Artificial Sequence
  hGH-Syn(gamma) 106-127 fusion protein
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGSQQEGVASKEKEEVAEEAQSGGD

SEQ ID NO: 104 - protein, 199 aa, Artificial Sequence
  GCSF-Syn119-140 fusion protein
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGSDPDNEAYEMPSEEGYQDYEPEA

SEQ ID NO: 105 - protein, 171 aa, Artificial Sequence
  hLeptin-Syn119-140 fusion protein
MVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGSDPDNEAYEMPSEEGYQDYEPEA